(19) United States
(12) Patent Application Publication
(10) Pub. No.: US 2025/0042946 A1
(43) Pub. Date: Feb. 6, 2025

(54) Biological Production of Histidine-Rich Peptides

(71) Applicant: Oakgrove Bio LLC, Wilmington, DE (US)

(72) Inventor: Pierre Eugene ROUVIERE, Wilmington, DE (US)

(73) Assignee: Oakgrove Bio LLC, Wilmington, DE (US)

(21) Appl. No.: 18/716,896

(22) PCT Filed: Dec. 7, 2022
(86) PCT No.: PCT/US2022/081119

§ 371 Date: Jun. 5, 2024

Related U.S. Application Data
(60) Provisional application No. 63/287,501, filed on Dec. 8, 2021.

(51) Int. Cl. C07K 7/08; C07K 5/107; C07K 7/06

(52) U.S. Cl. CPC C07K 7/08; C07K 5/1016; C07K 7/06; C07K 2319/31

(57) ABSTRACT

Self-aggregating peptides (SAPs) and their inclusion in the design of insolubility fusion partners (IFPs) and insoluble immunogenic epitopes (IIPs) are provided herein. The solubility of the SAPs and the peptides in which they are included can be controlled primarily by modulating the pH. Also disclosed herein is a method of recovering and purifying the peptides based on this pH modulation. As such, the peptides and methods described herein enable the production of bioactive or immunogenic peptides fused these SAPs.

### CROSS-REFERENCE TO RELATED APPLICATIONS

This claims the benefit of the filing date of U.S. Provisional Application No. 63/287,501, filed Dec. 8, 2021, the entire content of which is incorporated by reference herein.

### FIELD OF THE INVENTION

This invention relates to the field of biological production of peptides and proteins. In particular, it describes a method for low-cost production of histidine-rich peptides and can find broad applications from materials to medicine.

### BACKGROUND OF THE INVENTION

The last twenty years have seen an explosion of research on thousands of bioactive and structural peptides (10-100 amino acids (aa) in length) for many health applications including antimicrobials, vaccines, scaffolds for tissue engineering and drug delivery, bioactives for medical nutrition and for the control of chronic diseases, and growth factors for cell culture media. However, despite their great potential, relatively few peptides are being investigated beyond academic research in part because of the prohibitive cost of peptide chemical synthesis for both research and commercialization [Lax 2019]. In addition to their applications to health, many peptides demonstrate properties that would be applicable to personal care, agriculture or materials.

The majority of peptides are made by chemical synthesis. Short synthetic peptides (usually less than 10 aa in length) can be produced in liquid phase at a cost of at least about $1000 per kg per amino acid [Lax 2019]. Peptides longer than 10 aa are usually produced by solid phase synthesis. Their cost grows exponentially as the length of the peptide increases because of the frequency of incorrect additions at each elongation cycle [Isidro-Llobet 2019]. The removal of these slightly different contaminating peptides requires complex and expensive chromatography purification steps [Kota 2019]. The commercial price for research quantities of, for example, 5 mg of a 30-aa peptide at 90% purity is around $350, making it very expensive to test hundreds of variants for efficacy optimization. At commercial scales, the cost of manufacturing long peptides is still very high (greater than $100 to $1000/g). Only life-sustaining therapies can bear such cost for peptides longer than 35 aa. The high cost of the peptides also limits their delivery to injections and currently excludes oral and intranasal deliveries that require much greater quantities of therapeutic agent [De Palma 2015], beyond typical production scales that rarely exceeds a few hundred kg. Only the HIV peptide drug, Enfurvirtide, is produced at more than one ton. Thus, there exists a need for lower cost peptide manufacturing processes for the broad development of new peptide applications.

Recombinant production of proteins is a mature technology with low cost of manufacturing that enables their use beyond the medical field in industrial applications such as home care, food and feed processing, and environmental clean-up. The biological production of peptides could also be an alternative to costly chemical synthesis, however it is more challenging than that of proteins. Peptides often have no secondary or tertiary structure, making them readily accessible to proteases and prone to proteolytic degradation, whether produced inside or outside the cell. Attempts to produce peptides biologically have been documented for many years in dozens of publications [reviewed in Wibowo 2019]. However, most production methods described are, at the onset, unlikely to be commercially viable because of low titers and expensive purification processes that increase the cost of manufacturing to within the same range as biopharmaceutical proteins and synthetic peptides (i.e., greater than about $100s/g).

Currently, attempts to produce peptides biologically typcially use strategies and tools adapted for the production of soluble and folded proteins (enzymes, antibodies, signaling proteins, etc.) for which tertiary and quaternary structure, proper disulfide bond formation, or post-translational modifications are essential for activity. These expression strategies aim to maintain the nascent protein chains in a folding-compatible state by preventing either their aggregation (i.e., precipitation of unfolded proteins) or their proteolytic degradation. This is done by fusing the peptide of interest (POI) to be produced to a highly soluble protein [Zorko 2014, Ingham 2007, Parachin 2012, Wibowo 2017]. Such solubility fusion partners include thioredoxin, cellulose or chitin-binding domains, glutathione S-transferase, maltose binding protein, small ubiquitin-like modifier, and Fc antibody fragment. Once expressed, the soluble fusion protein must be separated from the soluble host proteins and other soluble molecules by chromatographic steps that are costly and difficult to scale up. After separation, the peptide must be cleaved from its soluble fusion partner, most often with enzymes of high specificity but low activity that are also expensive and difficult to scale up. Furthermore, most of these tools have been designed and are available only for research scale and not for commercial scale production [Bell 2013]. Finally, these “soluble expression” approaches often yield low peptide titers, possibly because of the accessibility of the peptide chain to cytoplasmic proteases and peptidases.

As an alternative to the soluble production of peptides with solubility fusion partners, insolubility fusion partners (IFPs) have been used to drive the expression of peptides into insoluble precipitates termed inclusion bodies (IBs) [Yang 2018]. In most cases, the IFP used is a small protein (less than 100 aa) that fails to express solubly in E. coli (i.e., “efficiently misfolding”), such as the 136-aa ketosteroid isomerase (KSI) [U.S. Pat. Nos. 5,648,244; 8,796,431), the 104-aa onconase [Gaglione 2019], a 162-aa dihydrofolate reductase [U.S. Pat. No. 7,595,173], a 126-aa truncated dehalogenase [U.S. Pat. No. 7,595,173], a subunit of a 138-aa human osteogenic protein [U.S. Pat. No. 7,749,731], a 105-aa beta-galactosidase fragment [U.S. Pat. No. 6,037,145] or a 174-aa hemolysin [U.S. Pat. No. 10,443,081]. However, the use of such proteins is only relevant at small scale for research because, once precipitated as inclusion bodies, the IFP-POI fusion must be solubilized by a chaotrope (guanidinium hydrochloride or urea) at very high concentration (e.g., 8M urea at about 480 g/L). After solubilization, most peptides need to be purified with multiple chromatographic steps such as affinity chromatography, ion exchange chromatography or gel filtration chromatography. These steps add to cost and are difficult to scale up.

Insolubility fusion partners consisting of small insoluble proteins (greater than 100 aa) also have the disadvantage of being quite large compared to the peptide of interest to be produced (20 to 60 aa) and, as such, constitute a waste product that increases the cost of manufacturing. It is thus desirable to use insolubility fusion partners as small as possible for commercial scale production.

Decarolis et al. describe small IFPs, typically less than 30 aa, that derive from natural storage proteins and lead to the insoluble production of some peptides [U.S. Pat. Nos. 7,662,913, 7,732,569]. Similarly, Williams et al. describe small 30-aa insolubility fusion partners with a hydrophobic core [U.S. Pat. No. 9,951,368]. Cheng et al. [U.S. Pat. No. 7,794,979] describe a family of IFPs based on using catenated segments derived from the self-assembling peptide P11-II from Aggeli et al. [Aggeli 2003]. The chemically synthesized and amidated peptide P11-II (CH3CO-Gln-Gln-Arg-Phe-Gln-Trp-Gln-Phe-Glu-Gln-Gln-NH2) is shown to have the propensity to self-assemble in aqueous environments into higher structure beta-sheets. P11-II (also referred as P11-2 or DNP1) was designed to alternate polar and hydrophobic amino acids and to promote the anti-parallel alignment of successive copies of the peptide. A positively charged arginine at position 3 and a negatively charged glutamic acid at position 9 are expected to facilitate the alignment of antiparallel copies by pairing and neutralizing their opposite charges.

A few other small insolubility fusion partners have been evaluated for the insoluble production of folded proteins. Wu et al. describe a 16-amino acid fibril-forming peptide, ELK16 [Wu 2011]. Hayakawa et al. describe the use of a fragment of “4AaCter”, a 55-amino acid sequence derived from an insoluble bacterial toxin to produce a Treponema antigenic protein [Hayakawa 2010].

The insolubility fusion partners described above have been selected or designed to promote the insoluble accumulation of proteins and peptides in the cytoplasm of a microbial host. However, there has been little work reported in the scientific and patent literatures to adapt them to a downstream process that results in the removal of host contaminants present in the inclusion body preparation, especially in processes that can be scaled up for the commercial production of peptides. U.S. Pat. No. 7,951,559 reports a process for the removal of insolubility fusion partners based on their oxidative intermolecular cross-linking using IFPs engineered to include multiple cysteines as described in U.S. Pat. No. 7,678,883.

U.S. Pat. No. 9,200,306 describes a process that increases the purity of a peptide to be produced by fusing it between an insolubility fusion partner (ELK16) and a solubility partner (thioredoxin), each of which having to be cleaved enzymatically using two distinct cleavage systems, a process that is expensive and slow. Zhao et al. expanded on this complex approach using additional small insolubility fusion partners [Zhao 2016]. Therefore, the need for an improved process for the recombinant production of peptides at low cost commercial scale still exists.

U.S. Pat. No. 9,062,312 describes how the pH manipulation of fusion peptides can be used for the coating of a silica surface, first by solubilizing the positively charged inclusion bodies at pH 12 and then by redepositing the fusion peptide on a negatively charged silica surface at neutral pH. The object of that disclosure is the modification of material surfaces aiming to avoid the cleavage of the insolubility fusion partner from the peptide of interest but not the recovery and purification of the peptide.

Insoluble peptides can also be used to display immunogenic epitopes. For example, U.S. Pat. No. 10,596,238 describes a 33-amino acid multifunctional peptide in which the sequence of one self-assembling peptide is linked to that of an immunogenic peptide. The peptide is shown to assemble in fibrils postulated to display a multiplicity of immunogenic epitopes; however, this disclosure is only relevant to chemically synthesized peptides. The chemical production of such immunogenic self-assembling peptides has several shortcomings. First, the chemical synthesis is limited to short amino acid chains as reflected in the claims of U.S. Pat. No. 10,596,238 (less than 40 amino acids for the self-assembling peptide and less than 20 amino acids for the immunogenic peptide). Second, because of the high cost of peptide synthesis, only simple architectures can be envisioned in which the immunogenic peptide is simply fused to a self-assembling peptide, leaving the immunogenic peptide with a free N-terminus, unprotected from proteolysis. Third, having a free end, the displayed epitope may not be constrained appropriately and may not be as immunogenic as in its natural context. Finally, the cost of production of such a vaccine product, like the 32-amino acid peptides recited in U.S. Pat. No. 10,596,238, will be very high and may limit the broad use of such a vaccine.

Biological approaches have been reported for the production of multi-epitope peptide vaccines. A straightforward approach is based on the expression of a fusion peptide composed of a structural protein fused to a string of immunogenic epitopes. Burkhard and collaborators [Kaba 2012, El Bissati 2014, U.S. Pat. No. 8,575,110] have developed a system for the production of self-assembling nanoparticles based on a combination of pentameric and trimeric coiled-coil segments interspersed with immunogenic epitopes. These immunogenic peptides are produced as inclusion bodies that must be solubilized in 9M urea, purified by Ni-agarose affinity agarose in the presence of 9M urea. The nanoparticles are then allowed to self-assemble through the progressive removal of the urea by dialysis. This process is difficult to scale up and costly due to the very high concentration of urea required, the affinity chromatography and the slow dialysis process required for the self-assembly of the nano particles.

More recently, fusions of epitopes to flagellin subunits with the ability to form nanoparticles have been used [Karch 2017, El Bissati 2017, Negahdaripour 2020, U.S. Pat. No. 10,245,318]. As was seen previously, however, the fusion protein is expressed insolubly as inclusion bodies in the cells and needs to be resolubilized in 9 M urea prior to its purification under denaturing conditions by Ni-agarose affinity chromatography. Similarly, Ito et al. report the production of a multi-epitope vaccine using immunogenic epitopes interspersed with alpha-helical domains but it also involves solubilization with 8M urea and metal-chelation chromatography [Ito 2017].

The problem to be solved is to provide a more economical process for the production of peptides at commercial scale, with a higher level of purity without expensive purification steps. More specifically, any improvement should aim at eliminating or greatly reducing the need for costly chemicals or chemicals used in very high concentration, chromatographic steps as well as expensive and inefficient enzyme treatments. The production of peptides at lower cost and large scale should enable the broader use and applications of new peptide products.

### SUMMARY OF THE INVENTION

The problems in the art presented above are solved through the discovery of new self-aggregating peptides (SAPs) and their inclusion in the design of insolubility fusion partners (IFPs) and insoluble immunogenic epitopes [IIPs], the solubility of which can be controlled primarily by modulating the pH. Also disclosed herein is a recovery and purification method based on manipulating the pH to take advantage of the solubility properties of the insolubility fusion partner. The invention also describes a method for the production of bioactive or immunogenic peptides that are fused to SAPs, in particular for the production of peptide vaccines. Finally, the low cost of production of peptides described herein is expected to enable the broader use of peptides in material, human, and animal nutrition and personal care applications.

The self-aggregating peptides (SAPs) and insolubility fusion partners (IFPs) disclosed herein are preferably designed to have the following properties:
1) be insoluble at neutral pH, ideally between pH 6 and pH 8;
2) be soluble at acidic pH, ideally between pH 5 and pH 2;
3) be soluble at alkaline pH, ideally between pH 9 and 12;
4) switch between solubility behaviors reversibly and rapidly following changes of pH;
5) impart solubility properties to a fusion peptide in which the insolubility fusion partner is fused to a peptide of interest;
6) be as short as possible to minimize the amount of fusion partner, which ultimately is a waste product, compared to the amount of peptide of interest produced.

Such properties should enable the development of a purification process based on pH changes in which the solubility properties of the fusion peptide and of the fusion partner are opposite of that of small molecules and macromolecules of the microbial host.

Accordingly, an aspect of the invention features a criterion (Criterion 1) for the design of novel peptides rich in Histidine or Tyrosine that have useful solubility properties. To meet Criterion 1, a peptide comprising 2n or 2n+1 amino acids must include n−1 or more amino acids that are either His or Tyr, wherein n is equal or greater than 3 and wherein said peptide exhibits self-aggregating properties at neutral pH.

Additionally, provided herein is a peptide comprising 2n or 2n+1 amino acids wherein n−1 or more amino acids are either a His or a Tyr, wherein n is equal or greater than 3, and wherein the His residues are located on the polar face and the Tyr residues are located on the hydrophobic face of a designed beta-strand [Smith 1997]. Such peptides are expected to have a propensity to form antiparallel beta-sheets.

Additionally, the invention provides a peptide comprising 2n or 2n+1 amino acids wherein n−1 or more amino acids are either a His or a Tyr, wherein n is equal or greater than 3, and wherein the His residues and the Tyr residues are located on opposite sides of a designed alpha-helix with a seven-fold periodicity [Woolfson 2010]. Such peptides are expected to have a propensity to form coils of alpha-helices.

The invention provides examples of self-aggregating peptides (SAPs) with amino acid sequences that fulfill Criterion 1 and have the desired solubility properties. Some of these SAPs are the basis for engineering insolubility fusion partners (IFPs) that allow the biological production of peptides of interest (POIs) as inclusion bodies inside the cytoplasm of a host cell. They also enable the separation of the POI from contaminating molecules from the expression host in a process based on pH changes and physical separations.

In some embodiments, the SAPs are biologically-produced, which, as one having ordinary skill in the art would readily appreciate means that the SAPs are expressed and extracted from a living organism, such as a microbial organism (e.g., E. coli).

The invention also provides the design of insolubility fusion partners (IFPs) that comprise SAPs by following the formula:

[SAP]-[[Spacer]-[SAP]]m

wherein SAP represents a peptide with self-aggregation properties and its amino acid sequence follows the composition described in Criterion 1. The spacers connecting the self-aggregating peptides are designed to facilitate the formation of intramolecular antiparallel beta-sheets or coils of alpha helices and enhance the insolubility of the IFP at neutral pH. Accordingly, the invention provides an insolubility fusion partner comprising the structure [SAP]-[[Spacer]-[SAP]]m where;
a) SAP is a self-aggregating peptide following Criterion 1;
b) the spacer is a peptide having from 1 to 50 amino acids;
c) m is an integer from 0 to 10;
   and where said peptide exhibits self-aggregating properties at neutral pH between pH 6 and pH 8.

In another embodiment, the invention provides the fusion of insolubility fusion partners (IFPs) to peptides of interest (POI) via cleavable linkers (cLNK) according to various fusion peptide architectures such as, but not limited to:

IFP-cLNK-POI;

POI-cLNK-IFP;

IFP-[cLNK-POI]n;

[POI-cLNK]n-IFP;

IFP-cLNK-POI-cLNK-IFP;

IFP-[cLNK-POI-cLNK-IFP]n; and

wherein IFP represents one or multiple different insolubility fusion partners, POI represents one or multiple different peptides of interest, cLNK represents one or multiple different cleavable linkers and n is a number greater than 1.

In a preferred embodiment, the IFPs incorporate one or multiple copies of self-aggregating peptides with sequences selected from SEQ ID NO: 2 to SEQ ID NO: 35 described in Table 1. Further, as with the SAPs, the IFPs are biologically-produced in some embodiments.

In another embodiment, provided herein is a method for the production, the purification, and the recovery of a POI expressed in an insoluble form in a microbial host using an IFP designed as described above. This method for producing and recovering a POI includes the steps of:
a) providing a genetically engineered microbial host cell that expresses a genetic construct encoding the amino acid sequence of a fusion peptide, wherein said fusion peptide comprises:
   i) an insolubility fusion partner that is insoluble at neutral pH and soluble at acid pH;
   ii) a linker comprising a cleavage site; and
   iii) a peptide of interest that is soluble at neutral pH;
b) growing the microbial host under conditions wherein said fusion peptide is produced in an insoluble form in the host cytoplasm;
c) recovering the insoluble fusion peptide of step b);
d) solubilizing the recovered insoluble fusion peptide of step c) in an acidic medium;
e) recovering the solubilized fusion peptide of step d);
f) adjusting the composition of the solution containing the solubilized fusion peptide to be appropriate for an effective cleavage treatment;
g) cleaving the insolubility fusion partner from the peptide of interest; and
h) altering the pH to precipitate the insoluble fusion partner and recovering the peptide of interest in soluble form.

The peptide of interest (POI) to be produced may be a peptide for therapeutic applications such as antimicrobials, vaccines, scaffolds for tissue engineering and drug delivery or materials, bioactives for medical or veterinary nutrition and the control of chronic diseases and growth factors for cell culture media. Other applications include personal care for skin and hair, affinity peptides to bind to a molecule, a receptor, a cell, a mineral or a material.

In another embodiment, the IFP is not cleaved off from the POI and it may be an integral part of the end product. This can be used in many applications ranging from self-assembling functionalized materials for tissue engineering and drug delivery, nanotechnology, affinity media, modification of biological surfaces and materials.

This concept can be generalized for the design of insoluble peptides incorporating SAPs and biologically active peptides, in particular for the biological production of peptide vaccines displaying a multiplicity of immunogenic epitopes to increase the immune response. The biological production of such immunogenic fusion peptides can be advantageous for low cost production and for the production of insoluble immunogenic structures too long to be produced by chemical synthesis.

Also provided herein are bioactive or immunogenic fusion peptides that include a POI epitope according to the formula:

[[SAP-CON-]m-POI-[CON-SAP]n-CON]p

wherein:
p is an integer greater than 0;
m is an integer including 0, wherein in any repeating segment represented by p, m can be the same integer or m can be a different integer from any other repeating segment represented by p;
n is an integer including 0, wherein in any repeating segment represented by p, n can be the same integer or n can be a different integer from any other repeating segment represented by p;
SAP is a self-aggregating peptide and may represent more than one self-aggregating peptide amino acid sequence;
POI is a peptide of interest and may represent more than one epitope amino acid sequence;
CON is a connecting peptide and may represent more than one connector amino acid sequence; and wherein the last connector may be omitted.

In some embodiments, m is an integer from 0 to 3, n is an integer from 0 to 3, and p is an integer from 1 to 8. In such embodiments, within any repeating segment represented by p, m and/or n can be the same integer or a different integer from any other repeating segment represented by p. In yet other embodiments, m is an integer from 0 to 2, n is an integer from 0 to 2, and p is an integer from 1 to 5 with m and/or n being the same integer or a different integer between any two repeating segments represented by p.

Also provided herein is a method for producing an insoluble immunogenic or bioactive peptide that includes the steps of:
a) genetically engineering a microbial host to include a nucleic acid segment coding for an immunogenic or bioactive peptide that is insoluble at neutral pH and soluble under acidic conditions, wherein said immunogenic or bioactive peptide has the architecture [[SAP-CON-]m-POI-[CON-SAP]n-CON]p described above;
b) growing the genetically engineered host cell under conditions where the genetic construct is expressed and the encoded immunogenic or bioactive peptide is produced in an insoluble form in the cytoplasm of the host cell;
c) recovering the insoluble peptide after physical separation from soluble host cell components;
d) subjecting the insoluble immunogenic or bioactive peptide to an aqueous medium having a pH at which said insoluble immunogenic or bioactive peptide becomes soluble;
e) recovering the solubilized immunogenic or bioactive peptide in the aqueous phase after physical separation of insoluble host cell components;
f) adjusting the composition of the solution containing the immunogenic or bioactive peptide to a neutral pH at which said peptide becomes insoluble;
g) recovering the insoluble immunogenic or bioactive peptide after physical separation.

### BRIEF DESCRIPTION OF THE FIGURES

FIG. 1 is a picture of a polyacrylamide gel electrophoresis of the insoluble fraction of E. coli extracts expressing various Insolubility Fusion Partners (IFPs) designed to incorporate multiple Self Aggregating Peptide (SAPs). The results show that IFPs based on His and Tyr-rich SAPs express in E. coli and accumulate insolubly in the cell.

FIG. 2 is a picture of a polyacrylamide gel electrophoresis of the insoluble fraction of extracts of E. coli expressing various IFP fused to peptide of interest (POI). FIG. 2 reveals that fusions of various POIs to IFPs based on His and Tyr-rich SAPs express well in E. coli and accumulate insolubly in the cell.

FIG. 3 is a picture of a polyacrylamide gel electrophoresis of the insoluble fraction of extracts of E. coli expressing various IFP fused to peptide of interest (POI) before and after an acid clean-up process that involves the solubilization of the fusion, its separation from insoluble denatured proteins and membranes and its re-insolubilization at neutral pH. It shows the increase purity of the fusion peptides after acid clean-up.

FIG. 4 is a picture of a polyacrylamide gel electrophoresis of the pH-based purification process for two prototypical peptides, Lunasin and Apidaecin. Panel A shows the purification of Lunasin from a fusion to IFP534. Following acid clean-up and acid-cleavage of the fusion IFP534-Luna, (MW=12.4 kDa) the Lunasin peptide (MW=5.6 kDa) is recovered in the soluble fraction at high purity (lane 5) while IFP534 (MW=6.4 kDa) remains in the insoluble fraction (lane 6). Panel B shows the purification of Apidaecin from a fusion to IFP221. Following acid clean-up and acid-cleavage of the fusion IFP221-Api, (MW=10.6 kDa) the Apidaecin peptide (MW=2.7 kDa) is recovered in the soluble fraction at high purity (lane 5) while IFP221 (MW=6.8 kDa) remains in the insoluble fraction (lane 6).

FIG. 5 is an illustration showing exemplary embodiments of structures that can be engineered to produce insoluble peptides in which a peptide of interest (POI) is displayed as a fusion to one or more self-aggregating peptides (SAP). These structures can be particularly useful when the POI is an immunogenic epitope for the production of vaccines or for biomaterials. Structures 1a and 1b represent the N- and C-terminus fusions of a POI to a single SAP. Structures 2a and 2b represent the N- and C-terminus fusions of a POI to two SAPs connected by a spacer. Structures 3, 4 and 5 represent the fusion of a POI “sandwiched” between one or two SAPs.

FIG. 6 is an illustration showing exemplary embodiments of structures that can be engineered to produce insoluble peptides in which multiple POIs to be displayed, identical or different, are fused to one or more SAPs. Structures 6 and 7 represent the fusions of multiple POIs interspersed between one or two SAPs. The length of the linkers and the spacers on the drawing does not reflect the actual length of their amino acid sequence. Structure 8 represents a specific example of Structure 7 in which five different immunogenic epitopes are interspersed with six SAPs.

FIG. 7 is a picture of a polyacrylamide gel electrophoresis of the pH-based purification process for four Insoluble immunogenic Peptides (IIPs). IIP006 (18.1 kDa) is an IIP comprising 5 Ova1 epitopes sandwiched between 6 copies of SAP162, IIP011 (9.7 kDa) is an IIP comprising 1 Ova1 epitope sandwiched between 4 copies of SAP081, IIP014 (8.5 kDa) is an IIP comprising 1 Ova1 epitopes sandwiched between 4 copies of SAP080 and IIP019 (21 kDa) is an IIP comprising 5 different epitopes sandwiched between 6 copies of SAP081. Lanes 3 show the increase of purity of the IIPs following the acid-based clean-up process enabled by the solubility properties of their SAPs. Lanes 4 show the further increase in purity of the IIPs and their decrease in molecular weight following the acid cleavage of the N-terminal and C-terminal ends of the IIPs, again enabled by the solubility properties of their SAPs.

FIG. 8 is an illustration showing exemplary embodiments of catenated structures that can be engineered to produce Insoluble Immunogenic Peptides (IIPs) in which a POI to be displayed singly is fused to one or more SAPs. Structures 9 and 10 represent variations of Structures 6 and 7 respectively with the addition of cleavable spacers at their N- and C-termini. The cleavage of catenated precursors at the level of cleavable linkers or spacers (white diamonds representing cleavage sites) yields multiple copies of shorter insoluble peptides of structures 3 and 1a in FIG. 1. The lengths of the linkers and the spacers on the drawing do not reflect the actual length of their amino acid sequences.

FIG. 9 is a picture of a polyacrylamide gel electrophoresis of the production of short IIPs from larger catenated IIPs. IIP003 (16.2 kDa) is an IPP comprising 3 Ova1 epitopes sandwiched between 6 copies of SAP162. IIP015 (14.2 kDa) is an IPP comprising 3 Ova1 epitopes sandwiched between 6 copies of SAP234. Lanes 3 show their increase in purity following the acid clean-up process. Lanes 4 show the recovery of smaller IIPs (5.0 kDa and 4.4 kDa respectively) comprising a single Ova1 epitope sandwiched between 2 SAPs.

### DESCRIPTION OF THE BIOLOGICAL SEQUENCES

SEQ ID NO: 1 is the amino acid sequence of a peptide composed of alternating histidine and tyrosine residues following the formula:

(Tyr)p-(His-Tyr)n-(His)q (Formula 1),

wherein:
p is a number equal to 0 or 1;
q is a number equal to 0 or 1;
n is a number equal to or greater than 3.

SEQ ID NO: 2 is the amino acid sequence of an 11-aa peptide composed of alternating histidines and tyrosines (HYHYHYHYHYH). SEQ ID NO: 3 is the amino acid sequence of a 13-aa peptide composed of alternating histidines and tyrosines (HYHYHYHYHYHYH). SEQ ID NO: 4 is the amino acid sequence of 9-aa peptide composed of alternating histidines and tyrosines (HYHYHYHYH). SEQ ID NO: 5 is the amino acid sequence of 7-aa peptide composed of alternating histidines and tyrosines (HYHYHYH). SEQ ID NO: 6 to SEQ ID NO: 35 are the amino acid sequences of representative peptides that derive from SEQ ID NO: 2, SEQ ID: 3, SEQ ID NO: 4 or SEQ ID NO:5 by substitution of some of the His or the Tyr residues by amino acids other than His or Tyr and that fulfill Criterion 1. They are described in Table 1.

TABLE 1
Representative His and Tyr Rich Peptides

| SEQ ID NO | Amino Acid sequence | SAP Name |
|---|---|---|
| SEQ ID NO: 2 | HYHYHYHYHYH | SAP119 |
| SEQ ID NO: 3 | HYHYHYHYHYHYH | SAP210 |
| SEQ ID NO: 4 | HYHYHYHYH | SAP231 |
| SEQ ID NO: 5 | HYHYHYH | SAP232 |
| SEQ ID NO: 6 | QQHYHYHQQ | SAP234 |
| SEQ ID NO: 7 | HQHYHYHQH | SAP238 |
| SEQ ID NO: 8 | QHYHYHYHQ | SAP241 |
| SEQ ID NO: 9 | QQHYHYHYHQQ | SAP080 |
| SEQ ID NO: 10 | QQEYHYHYRQQ | SAP081 |
| SEQ ID NO: 11 | QQEYHYHYHYRQQ | SAP221 |
| SEQ ID NO: 12 | HQEYHYHYRQH | SAP162 |
| SEQ ID NO: 13 | QQHYHAHYHQQ | SAP186 |
| SEQ ID NO: 14 | QQRYHYHYEQQ | SAP091 |
| SEQ ID NO: 15 | HQRYHYHYEQH | SAP092 |
| SEQ ID NO: 16 | QQYHYHYQQ | SAP240 |
| SEQ ID NO: 17 | QQHYHYHYHYHQQ | SAP217 |
| SEQ ID NO: 18 | HQHYHYHYHQH | SAP180 |
| SEQ ID NO: 19 | QYEYHYHYRYQ | SAP120 |
| SEQ ID NO: 20 | HYEYHYHYRYH | SAP138 |
| SEQ ID NO: 21 | QYEYEQRYRYQ | SAP137 |
| SEQ ID NO: 22 | HYEYEYRYRYH | SAP139 |
| SEQ ID NO: 23 | QQHYHFHYHQQ | SAP061 |
| SEQ ID NO: 24 | HQHYHAHYHQH | SAP187 |
| SEQ ID NO: 25 | QHYHAHYHQ | SAP242 |
| SEQ ID NO: 26 | QQEYHFHYRQQ | SAP062 |
| SEQ ID NO: 27 | HQEYHFHYRQH | SAP063 |
| SEQ ID NO: 28 | HQEYHVHYRQH | SAP188 |
| SEQ ID NO: 29 | HQEYHIHYRQH | SAP189 |
| SEQ ID NO: 30 | QQHFHFHFHQQ | SAP065 |
| SEQ ID NO: 31 | HQEFHYHFRQH | SAP068 |
| SEQ ID NO: 32 | HQEFHFHFRQH | SAP064 |
| SEQ ID NO: 33 | HQEYHCHYRQH | SAP095 |
| SEQ ID NO: 34 | QQHYHCHYHQQ | SAP093 |
| SEQ ID NO: 35 | QQEYHCHYRQQ | SAP094 |

SEQ ID NO: 36 to SEQ ID NO: 49 are the sequences of design elements for the construction of insolubility fusion partners, fusion peptides, and insoluble immunogenic peptides that include SAPs. They are listed in Table 2.

TABLE 2
Elements of Fusion Peptide Designs

| SEQ ID | Design | Amino acid |
|---|---|---|
| NO: | element | sequence |
| SEQ ID | Flexible | GGSGGSGG |
| NO: 36 | linker | |
| Not | Acid-cleavable | GGDP |
| assigned | linker | |
| Not | Acid-cleavable | DPGG |
| assigned | linker | |
| Not | Acid-cleavable | PDPG |
| assigned | spacer | |
| SEQ ID | Acid-cleavable | GGDPGG |
| NO: 37 | linker | |
| SEQ ID | Acid-cleavable | GGSGDPGSGG |
| NO: 38 | linker | |
| SEQ ID | Hexa-histidine | HHHHHH |
| NO: 39 | cap | |
| SEQ ID | Proline-rich | PGPGP |
| NO: 40 | cap | |
| SEQ ID | Lunasin | SKWQHQQDSCRKQL |
| NO: 41 | peptide | QGVNLTPCEKHIME |
| | | KIQGRGDDDD |
| | | DDDDD |
| SEQ ID | LL37 | LLGDFFRKSKEKI |
| NO: 42 | peptide | GKEFKRIVQRIK |
| | | DFLRNLVPRTES |
| SEQ ID | Apidaecin | GNNRPVYIPQPR |
| NO: 43 | peptide | PPHPR |
| SEQ ID | Tachyplesin | KWCFRVCYRGIC |
| NO: 44 | peptide | YRRCR |
| SEQ ID | OVA1 | SIINFEKL |
| NO: 45 | epitope | |
| SEQ ID | OVA2 | SQAVHAAHAEIN |
| NO: 46 | epitope | EAGR |
| SEQ ID | RS09 | APPHALS |
| NO: 47 | epitope | |
| SEQ ID | TPD | LMQYIKANSKFIGI |
| NO: 48 | epitope | PMGLPQSIALSSLM |
| | | VAQ |
| SEQ ID | PADRE | AKFVAAWTLKAA |
| NO: 49 | epitope | |

SEQ ID NO: 50 to SEQ ID NO: 117 are the amino acid sequences of insolubility fusion partners, fusion peptides and insoluble immunogenic peptides and the nucleotide sequences of the DNA fragments that they are encoded by.

TABLE 3
Insoluble Fusion partners, Peptide Fusions and Insoluble
Immunogenic Peptides Described in the Examples

| | | Amino acid | Nucleotide |
|---|---|---|---|
| Peptide | Peptide | sequence | sequence |
| Name | description | SEQ ID NO | SEQ ID NO |
| IFP221_PRG | IFP | SEQ ID NO: 50 | SEQ ID NO: 51 |
| IFP365 | IFP | SEQ ID NO: 52 | SEQ ID NO: 53 |
| IFP380 | IFP | SEQ ID NO: 54 | SEQ ID NO: 55 |
| IFP381 | IFP | SEQ ID NO: 56 | SEQ ID NO: 57 |
| IFP462 | IFP | SEQ ID NO: 58 | SEQ ID NO: 59 |
| IFP463 | IFP | SEQ ID NO: 60 | SEQ ID NO: 61 |
| IFP534 | IFP | SEQ ID NO: 62 | SEQ ID NO: 63 |
| IFP534_PRG | IFP | SEQ ID NO: 64 | SEQ ID NO: 65 |
| IFP538 | IFP | SEQ ID NO: 66 | SEQ ID NO: 67 |
| IFP540 | IFP | SEQ ID NO: 68 | SEQ ID NO: 69 |
| IFP562 | IFP | SEQ ID NO: 70 | SEQ ID NO: 71 |
| IFP563 | IFP | SEQ ID NO: 72 | SEQ ID NO: 73 |
| IFP462-Api | Fusion | SEQ ID NO: 74 | SEQ ID NO: 75 |
| IFP462-Luna | Fusion | SEQ ID NO: 76 | SEQ ID NO: 77 |
| IFP534-Luna | Fusion | SEQ ID NO: 78 | SEQ ID NO: 79 |
| IFP380-Api | Fusion | SEQ ID NO: 80 | SEQ ID NO: 81 |
| IFP380-Luna | Fusion | SEQ ID NO: 82 | SEQ ID NO: 83 |
| IFP381-Luna | Fusion | SEQ ID NO: 84 | SEQ ID NO: 85 |
| IFP534-Api | Fusion | SEQ ID NO: 86 | SEQ ID NO: 87 |
| IFP381-Api | Fusion | SEQ ID NO: 88 | SEQ ID NO: 89 |
| IFP221-Api | Fusion | SEQ ID NO: 90 | SEQ ID NO: 91 |
| IFP534-Tach | Fusion | SEQ ID NO: 92 | SEQ ID NO: 93 |
| IFP221-Apix2 | Fusion | SEQ ID NO: 94 | SEQ ID NO: 95 |
| IFP634-LL37 | Fusion | SEQ ID NO: 96 | SEQ ID NO: 97 |
| IFP-Api-003 | Split Fusion | SEQ ID NO: 98 | SEQ ID NO: 99 |
| IFP-Api-006 | Split Fusion | SEQ ID NO: 100 | SEQ ID NO: 101 |
| IFP-Luna-006 | Split Fusion | SEQ ID NO: 102 | SEQ ID NO: 103 |
| IIP003 | IIP | SEQ ID NO: 104 | SEQ ID NO: 105 |
| IIP006 | IIP | SEQ ID NO: 106 | SEQ ID NO: 107 |
| IIP004 | IIP | SEQ ID NO: 108 | SEQ ID NO: 109 |
| IIP011 | IIP | SEQ ID NO: 110 | SEQ ID NO: 111 |
| IIP014 | IIP | SEQ ID NO: 112 | SEQ ID NO: 113 |
| IIP015 | IIP | SEQ ID NO: 114 | SEQ ID NO: 115 |
| IIP019 | IIP | SEQ ID NO: 116 | SEQ ID NO: 117 |

Table 3 lists examples of peptides expressed in E. coli that are presented in the Examples. Their detailed structures are listed in tables in the Examples. The amino acid SEQ ID NOs refer to the entire amino acid of the peptides. The nucleotide SEQ ID NOs refer to a DNA fragment encoding each peptide. These fragments were cloned in frame in the Ndel and Xhol sites of expression plasmid pET-29b (+). The ATG codon of the Ndel site codes for the initiating Met of the peptides. For many of these, the nucleotide sequence of the DNA fragment includes non-coding DNA “stuffer” sequence at the 3′-end of the peptide coding sequence to enable DNA synthesis and cloning.

SEQ ID NO: 118 is the nucleotide sequence of the expression plasmid pET-29b (+). SEQ ID NO: 119 is the amino acid sequence of IFP100, a 2-SAP IFP that is included in split fusions.

### DETAILED DESCRIPTION OF THE INVENTION

### Abbreviations and Definitions

The following abbreviations are used herein: “SAP” for Self-Aggregating Peptide, “IFP” for Insolubility Fusion Partner, “POI” for Peptide of Interest, “CON” for Connector, “LNK” for Linker, “cLNK” for cleavable Linker, “cSpacer” for cleavable Spacer, “IB” for Inclusion Body, “IIP” for Insoluble Immunogenic Peptide, “SDS-PAGE” for Sodium Dodecyl Sulfate Polyacrylamide Gel Electrophoresis”, “Api” for Apidaecin, “Luna” for Lunasin. Peptide LL37 is often referred in the literature as LL-37. Acronyms are pluralized by addition of an s.

The amino acids are abbreviated with their three- and one-letter codes: Alanine-Ala-A; Arginine-Arg-R; Asparagine-Asn-N; Aspartic acid-Asp-D; Cysteine-Cys-C; Glutamine-Gln-Q; Glutamic acid-Glu-E; Glycine-Gly-G; Histidine-His-H; Isoleucine-IIe-I; Leucine-Leu-L; Lysine-Lys-K; Methionine-Met-M; Phenylalanine-Phe-F; Proline-Pro-P; Serine-Ser-S; Threonine-Thr-T; Tryptophan-Trp-W; Tyrosine-Tyr-Y; Valine-Val-V. Xaa may represent any amino acid.

The following abbreviations for model immunogenic peptide epitopes used herein are: “OVA1” and “OVA257-254” refer to the model epitope corresponding to amino acids 257 to 254 of chicken egg ovalbumin (GenBank accession reference: AUD54707.1); “OVA2” and “OVA323-339” refer to the model corresponding to amino acids 323 to 339 of chicken egg ovalbumin [Garulli 2011]; “TPD” refers to the chimeric major histo-compatibility class II epitope [Fraser 2014]; “PADRE” refers to a “pan HLA-DR reactive” epitope [Alexander 1994]; “RS09” and “APPHALS” refer to the Tol-like receptor TLR4 agonist epitope [Shanmugam 2012].

The term “biologically-produced” as used herein to refer to a peptide or a fusion peptide means that the peptide was encoded by a nucleic acid construct and expressed in a living organism, such as, but not limited to a bacterium (e.g., E. coli), a fungus, a plant cell, or an animal cell using any suitable molecular or genetic engineering technique.

The term “peptide” as used herein refers to chains of natural amino acids, not limited in length, but having no complex tertiary structure that would require a complex folding pathway. Peptides may have a secondary structure or even repetitive higher order structures (e.g. fibrils, helix bundles, proline helices, etc) that can form spontaneously and reversibly as a function of physico-chemical conditions. The term “polypeptide” as used herein refers to either a “peptide” as described above or to any other protein, including a globular protein, a membrane protein, a storage protein or a structural protein.

The terms “His-Tyr alternating peptide backbone” or “His-Tyr alternating backbone” or “His-Tyr alternating peptide” used interchangeably herein refer to peptides composed of His and Tyr residues wherein the His and Tyr residues alternate along the peptide chain to position all the His residues on the polar side of a predicted beta-strand and all the Tyr residues on the hydrophobic side of a predicted beta-strand. They can be described as fulfilling the formula:

(Tyr)p-(His-Tyr)n-(His)q (Formula 1)

wherein:
p is a number equal to 0 or 1;
q is a number equal to 0 or 1; and
n is a number equal to or greater than 3.

The amino acid sequences YHYHYH, HYHYHYH, YHYHYHYHYH or HYHYHYHYH fulfilling Formula 1 are examples of “His-Tyr alternating peptides”. In some embodiments, n is a number equal to or greater than 4, or equal to or greater than 5.

The term “peptide of interest” abbreviated POI refers herein to a peptide to be expressed, recovered and purified toward a specific use and that may require an insolubility fusion partner for its production. It may also refer to a peptide sequence with a specific functionality that is to be produced fused to one or more self-aggregating peptides. Functionalities of the POI include affinity, inhibition, activation, immunogenicity, binding, or targeting, modulation of charge, hydrophobicity or hydrophilicity, catalytic activity or mechanical properties.

The term “self-aggregating” used herein describes peptides that are insoluble in a solution of composition specific for a chosen pH, salt composition and concentration, solvent, temperature and peptide concentration. Self-aggregation can represent a non-specific precipitation of the peptide or preferably, the self-assembly of specific secondary or higher-order structures involving the formation of alpha-helix bundles, beta-sheets or other less common repetitive motifs. Such “self-assembling peptides” are special cases of self-aggregating peptides. Self-aggregating peptides are herein abbreviated “SAP”. As used herein, a peptide is deemed self-aggregating if it leads to the insoluble expression in the cytoplasm of a host cell of an insolubility fusion partner that comprises it.

The terms “fused”, “connected” and “linked” can be used interchangeably. The terms “connector”, “peptide connector” or “connecting peptide” abbreviated “CON” refer to an amino acid sequence that connects the various elements of the fusion peptide. Typically connectors will link two or more SAP's together, or an SAP and a POI. Peptide connectors will typically be classified as a “linker” or a “spacer” as defined herein.

The term “linker”, abbreviated “LNK”, refers to a stretch of amino acids connecting a POI to the C-terminus or the N-terminus of an IFP, connecting two POIs or connecting two SAPs. Linkers may be of any length and as short as one amino acid. They may include sequences that facilitate expression and recovery of the POIs, in particular they may include a cleavage site. Linkers that include a cleavage site are abbreviated “cLNK”.

The term “spacer” refers herein to a special type of linker, typically 1-50 amino acids in length, that separates SAP motifs to allow the formation of three-dimensional structures leading to greater insolubility. Such spacers may have the properties of providing flexibility, rigidity or bending between two SAP motifs. In certain embodiments, spacers can be “turns”. Spacers may also include a cleavable sequence, for example the acid labile Asp-Pro sequence such as in the cleavable spacer Pro-Asp-Pro-Gly. Spacers that include a cleavage site are labeled “cSpacer”. Spacers can also be engineered to have specific functionalities such as, but not limited to, enhancing solubility or insolubility, carrying charge, having binding affinity, displaying antigenicity or enzymatic activity.

The term “turn” refers herein to a special type of spacer or linker, being a small stretch of amino acids connecting two self-aggregating peptides to allow their spatial positioning and to facilitate the formation of a specific secondary structure. Turns are especially important in the formation of anti-parallel beta-sheets as well as for the formation of coiled-coil bundles of alpha-helices. In that context, turns can be as short as 1 amino acid. Turns starting with the sequence Pro-Xaa-Gly have been shown to be effective in promoting antiparallel beta sheets. Of particular interest herein are turns of the sequence Pro-Arg-Gly and Pro-Glu-Gly.

The term “insolubility fusion partner”, abbreviated “IFP”, represents a peptide segment that, when fused to another peptide or protein, leads to the accumulation of an insoluble fusion polypeptide when produced in an expression host, whether microbial, animal or plant. In this invention, IFPs are composed of one or more SAPs. Some IFPs are also endowed with the properties of pH-controlled solubility for the purification of POIs.

The term “fusion peptide” used herein represents a contiguous amino acid sequence comprising the amino acid sequences of one or more IFPs connected to the amino acid sequences of one or more POIs via a linker. The IFPs can be connected to a POI via the C-terminus or the N-terminus of the POI. In some embodiments, multiple copies of the same POI or of different POIs can be connected to one or more IFPs with various architectures.

The term “catenated” represents the stringing of multiple copies of a specific amino acid sequence.

The term “epitope” used herein refers to an amino acid sequence that is recognized by a receptor to promote a physiological response. An epitope can be an immunogen recognized by the immune system, specifically by antibodies, antigen-presenting cells, B cells, or T cells. As used herein, an epitope can also be an amino acid sequence recognized by other receptors such as a hormonal receptor, a metabolite receptor, a taste receptor, or other cell-surface receptors for targeting such as cancer cells, any sensory receptor or by sensors and modulators of gene expression. As used herein, an epitope can also be an affinity or a binding peptide sequence for any type of application such as material, mineral, biochemical or personal care applications. In the context of fusion peptides, an epitope can be considered and represented as a POI.

As used herein, the term “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

As used herein, the term “genetic construct” refers to a series of contiguous nucleic acids useful for modulating the genotype or phenotype of an organism. Non-limiting examples of genetic constructs include, but are not limited, to a nucleic acid molecule, an open reading frame, a gene, an operon, a plasmid, a genome and the like.

The term “vector”, as used herein, refers to a DNA or an RNA molecule such as a plasmid, a virus, a particle or other vehicle, that contains one or more heterologous or recombinant DNA sequences and that is designed to introduce genetic material in a host cell. The term “expression vector” refers to any vector that is effective in incorporating and in expressing heterologous DNA fragments in a cell. A cloning or expression vector may include additional elements, for example the expression vector may have two replication systems, thus allowing it to be maintained in two organisms, for example in human cells for expression and in a prokaryotic host for cloning and amplification. Any suitable vector can be employed that is effective for the introduction of nucleic acids into cells such that protein or polypeptide expression results, e.g. a viral vector or non-viral plasmid vector.

The term “expression host” refers herein to a living organism that is used to produce a molecule of interest. In this application, the expression host is an organism that can produce a peptide or a protein of interest having been transformed by a genetic construct comprising the gene or genes coding for the peptide or protein of interest as well as other nucleotide sequences carrying the information for the expression of the gene or genes. The expression host can be a plant or animal cell, or a microbe, eukaryotic or prokaryotic.

The term “inclusion bodies” abbreviated “IBs” and used herein refers to insoluble aggregates of proteins or polypeptides that are overexpressed inside the cytoplasm of an expression host. IBs may take the form of discrete particles, of diffuse aggregates or of fibrils inside the host cell. Upon disruption of the host cells, IBs can be recovered by a physical method based on a combination of density and size, for example by centrifugation or sedimentation, or their size, for example by filtration.

The term “cleavage site” refers to a stretch of amino acids that can be cleaved at a specific amino acid or at a specific amino acid sequence to separate a POI from the IFP or a POI from another POI. The cleavage can be either enzymatic or chemical. Examples of cleaving enzymes include Factor Xa, TEV protease, enterokinase, thrombin or inteins. Examples of chemical cleavage agents include hydroxylamine, cyanogen bromide, N-bromo-succinimide, 2-(2′-nitrophenylsulfonyl)-3-methyl-3-bromoindolenine, BNPS-skatole, and 2-nitro-5-thiocyanobenzoic acid as well as acids, such as hydrochloric, phosphoric or formic acids.

The term “acidic condition” refers herein to an aqueous environment with a pH less than 5. The term “alkaline condition” refers herein to an aqueous environment with a pH greater than 9.

The term “architecture” refers herein to the linear organization of the various elements that compose the insoluble peptide to be expressed, including the linear organization of the IFP and that of the POI, or of a combination of SAPs and POIs to be produced. In the description of a peptide architecture, it is implied that two SAPs are connected by a spacer and that an SAP and a POI or a POI and an SAP are connected by a linker.

### Production of Insoluble Fusion Peptides Comprising IFPs and SAPs

The present invention describes a set of new self-aggregating peptides and insolubility fusion partners that can be used for the economical biological production of soluble peptides in a plurality of applications. It also describes the production of insoluble peptides for biomaterial, nanotechnology and vaccine applications. As will be explained in further detail below, the insoluble fusion peptides described herein will include an IFP, and preferably, the IFP will include one or more SAPs. In some embodiments, the IFPs containing one or more SAPs are synthetically-produced; preferably, the IFPs containing one or more SAPs are biologically-produced.

As described above, most peptides currently commercially available in the art are produced by chemical synthesis, a method that has several significant shortcomings including, but not limited to high cost, limited applicability to only relatively short peptides and difficulty in scaling up beyond several hundreds of kilograms. Described herein is the biological production of peptides, which is an alternative to peptide chemical synthesis, especially for longer peptides. It is particularly advantageous when the peptide is produced in an insoluble form as inclusion bodies. However, as described in the art, the cost of peptide biological production is still prohibitively high thereby severely limiting many requiring large scale at low cost.

To lower the cost of peptide manufacturing, the methods described herein decrease or eliminate the need for chromatographic purification steps, expensive and slow cleavage enzymes, expensive chemicals, and chemicals used in high concentrations that may become expensive to use and to dispose of.

### Desirable Characteristics of Self-Aggregating Peptides and Insolubility Fusion Partners

A new biological production strategy has been designed based on novel and innovative self-aggregating peptides (SAPs) and insolubility fusion partners (IFPs), the solubility of which can be modulated by pH changes so that they can impart solubility properties opposite to that of many contaminating small molecules and macromolecules from the expression host. In general, the IFPs will include one or more SAPs in its peptide backbone. Further, the SAPs preferably will form secondary or higher ordered structures in neutral pH, such as, but not limited to alpha-helix bundles, beta-sheets, or other repetitive motifs. In some embodiments, the SAPs will self-assemble into antiparallel beta-sheets or coils of alpha helices at neutral pH. Such novel SAPs and resulting IFPs in which the novel SAPs are incorporated enable the use of physical separation methods for the peptide of interest from host contaminants such as sedimentation, centrifugation and filtration. These separation techniques are relatively cheap and scalable.

In preferred embodiments, the SAPs and IFPs comprising the SAPs that are described herein may have the following properties:
1) be insoluble at neutral pH, preferably between pH 6 and pH 8;
2) be soluble at acidic pH, preferably between pH 5 and pH 2
3) be soluble at alkaline pH, preferably between pH 9 and 12;
4) switch between solubility behaviors reversibly and rapidly following changes of pH;
5) impart their solubility properties to a fusion peptide in which the insolubility fusion partner is expressed as a fusion to a peptide of interest;
6) be as short as possible to minimize the amount of fusion partner, which ultimately is a waste product, compared to the amount of peptide of interest produced.

Such properties should enable the development of a purification process based on pH changes in which the solubility properties of the fusion peptide and of the fusion partner are opposite to that of small molecules and macromolecules of the microbial host. These properties can be screened after overexpression in a microbial host.

Modulation of a polypeptide charge can be done by including in the peptide sequence amino acids with a charged side chain. Amino acids having a charged side chain over pH ranging from 1 to 13 are arginine, lysine, histidine, aspartic acid, glutamic acid, cysteine and tyrosine. At neutral pH ranging from 6.5 to 7.5, the side chains of arginine and lysine display one positive charge, and the side chains of aspartic acid and glutamic acid display one negative charge. The side chains of histidine, cysteine and tyrosine are essentially uncharged in that pH range. Histidine displays one positive charge below pH 5.5, cysteine displays one negative charge above pH 9.5 and tyrosine one negative charge above pH 10.5. The side chains of other amino acids are not charged between pH 1 and 13. The change of charge and hydrophobicity at low or high pH is expected to create electrostatic repulsions, impact the ability for amino acids to form electrostatic interactions with amino acids of opposite charge and increase the overall polarity of the peptide. Furthermore, the change of charge weakens hydrophobic interactions in the case of tyrosine and histidine, thus decreasing the propensity of peptides to aggregate or form secondary structures and thus promote their solubility.

### Design of New Self-Aggregating Peptides Rich in his and Tyr-Criterion 1

As discussed above, the peptides of the present invention will include an IFP that comprises one or more self-aggregating peptides or SAPs. Moreover, it is preferably that these SAP peptides are responsive to pH by acquiring charges in a pH range outside neutrality, i.e. below pH 5 or above pH 9. As such, in a preferred embodiment, the SAP peptide will include a sufficient number of His and Tyr residues incorporated therein so that, for a peptide comprising 2n or 2n+1 amino acids, at least n−1 amino acids are either a Tyr or a His, wherein n is equal or greater than 3, and wherein said peptide exhibits self-aggregating properties at neutral pH (Criterion 1). For example, for a 6- or 7-aa peptide, at least 2 of the aa are either His or Tyr. For a 8- or 9-aa peptide, at least 3 of the aa are either His or Tyr. For a 10- or 11-aa peptide, at least 4 of the aa are either His or Tyr. For a 12- or 13-aa peptide, at least 5 of the aa are either His or Tyr, and so on.

Different sequence organizations of His and Tyr residues can be envisioned for insoluble peptides fulfilling Criterion 1 and lead to desirable and useful solubility properties. In a preferred embodiment, all the His residues face toward the polar side of a predicted beta-strand while all the Tyr residues face toward the hydrophobic side. In other terms, the His residues must be at even positions in the amino acid sequence of the peptide and the Tyr residues must be at odd positions, or vice versa, the His residues must be at odd positions in the amino acid sequence of the peptide and the Tyr residues must be at even positions.

### His-Tyr Alternating Peptides

The present invention provides a number of examples of SAPs having the characteristics described above. In addition to having Criterion 1, some embodiments of the SAPs may be derived from His-Tyr alternating peptides that fulfill Formula 1:

(Tyr)p-(His-Tyr)n-(His)q

where:
p is a number equal to 0 or 1;
q is a number equal to 0 or 1; and
n is a number equal to or greater than 3 (SEQ ID NO: 1).
   Non-limiting exemplary “backbone” peptides composed exclusively of His and Tyr include, e.g., SAP119 with the sequence HYHYHYHYHYH (11-aa, SEQ ID NO: 2), SAP210 with the sequence HYHYHYHYHYHYH (13-aa, SEQ ID NO: 3), SAP231 with the sequence HYHYHYHYH (9-aa, SEQ ID NO: 4) and SAP232 with the sequence HYHYHYH (7-aa, SEQ ID NO: 5). Other His-Tyr alternating “backbone” peptides can have sequences of any length if equal or longer than 6 amino acids and their derivatives are covered by the concept of this invention. In some embodiments, the peptides have sequences longer than 6 amino acids, or longer than 7 amino acids, or longer than 8 amino acids, or longer than 9 amino acids. For instance, in some embodiments, n is a number equal to or greater than 4, or equal to or greater than 5.

In a non-limiting exemplary embodiment, SAP119 carries no net charge at neutral pH, and the alternating His (polar) and Tyr (hydrophobic) are predicted to induce the formation of beta-sheets by secondary structure prediction software such as BETApro, PASTA or PepPro [Cheng 2005, Walsh 2014, Lamiable 2016]. In the most favorable anti-parallel structure, the His of different beta-strands located on the polar side of the anti-parallel beta-sheet form additional hydrogen bonds and the Tyr located on the hydrophobic side stabilize the structure by enhancing hydrophobic interactions. At an acidic pH where all the side chains of His residues are protonated, SAP119 carries a net positive charge of +6. These charges exert electrostatic repulsions between the beta-strands that are expected to destabilize the anti-parallel beta-sheet organization. Similarly, at an alkaline pH, where all the side chains of Tyr residues are deprotonated, SAP119 carries a net negative charge of −5. These charges exert electrostatic repulsions between the strands that are also expected to destabilize the anti-parallel beta-sheet organization.

According to the best of the inventor's knowledge, the biological production of alternating His-Tyr peptide backbones following Formula 1 (SEQ ID NO: 1) and/or their utility in a biological production process have not been investigated or described. In particular the integration of these Formula 1 peptides into the genetic engineering of larger peptide structures for the production of biologically-produced polypeptides such as IFPs or IIPs are novel and innovative. U.S. Pat. No. 9,162,005 and related patents list a peptide of sequence YYYYYHYHYHYHYHYH among a list of 342 16-aa peptides that purportedly could be used for the production of biogels for medical applications. However, these disclosures are centered around two amino acid combinations and show the actual production and functionality for only two “RADA” peptides while failing to teach the use of these peptides in larger fusion constructs. Recently, Diaz-Caballero et al. [ACS Catal. 11:595-607 (2021)] described the catalytic activity of chemically-synthesized short fibrils made of short His-Tyr alternating peptides that are N-terminally acetylated and C-terminally amidated and which assemble slowly in fibrils after several days. This publication did not include the incorporation of these sequences within larger polypeptides such as multi-SAP IFPs, fusions or IIPs. Thus the design and the utility of alternating His-Tyr peptide backbones of Formula 1 (SEQ ID NO: 1) in biological production as described herein is both novel and innovative.

### New Self-Aggregating Peptides Rich in his and Tyr Designed to Form Beta-Sheets

Insoluble, pH-responsive peptides rich in His and Tyr can be designed to form beta-sheets by positioning the His residues and Tyr residues on opposite sides of a putative beta-sheet. Operationally, these peptides can be viewed as deriving from His-Tyr alternating peptide backbones described in Formula 1 (SEQ ID NO:1) by the substitution of some histidine and tyrosine residues with amino acids other than histidine or tyrosine, while ensuring that the peptides retain a sufficient proportion of His and Tyr residues to fulfill Criterion 1. Such derivatives of peptides SAP119, SAP210, SAP231, SAP232 or of other alternating His-Tyr peptides longer than 6 amino acids, are expected to have different solubility properties, for example by modulating the insolubility at neutral pH, modulating the solubility at acidic or alkaline pHs, altering the kinetics of insolubilization and solubilization, modifying the minimum peptide concentration for aggregate formation, modifying the formation of higher order structures such as fibers, fibrils, or films, modifying the interactions of the insoluble aggregates with other macro-molecules or modifying the macroscopic properties of their solution.

In a non-limiting exemplary embodiment, a family of self-aggregating peptides (SEQ ID NO: 2 to SEQ ID 35; Table 1) was designed to control their solubility under a range of physical conditions that can be used in a purification process (pH, salt composition and concentration, hydrophobicity, temperature).

Derivatives of alternating His-Tyr peptides can be designed by substituting pairs of oppositely charged amino acids (Glu or Asp and Arg or Lys). For example, in the 11-aa SAP138 a Glu in position Xaa3 and an Arg in position Xaa9 can form attractive electrostatic forces between adjacent beta-strands that are expected to bring beta-strands in proximity. Furthermore, these electrostatic attractions facilitate the anti-parallel alignment of the beta-strands and may enhance the formation of more regular higher order structures.

Other uncharged polar amino acids with a propensity to form beta-sheets by forming hydrogen bonds such as Gln or Asn can be substituted for His on the polar face of a beta-strand and can be used to modulate the charge of the peptide and thus the solubility properties of the peptide as a function of pH. Other polar amino acids of lower hydrophilicity such as Ser or Thr may also substitute for His. Representative variants of SAP119, SAP210, SAP231 or SAP232 with Glu/Arg or Gln substitutions are listed in Table 1 as examples.

Other uncharged hydrophobic amino acids with side chains of various hydrophobicity such as Ala, Val, Met, Cys, IIe, Leu, Phe or Trp can be substituted for tyrosines on the hydrophobic face of a beta-strand and can be used to weaken or strengthen the hydrophobic interactions between antiparallel beta-strands and thus modulate the solubility properties of the peptide as a function of pH. Representative variants of SAP119, SAP210, SAP231 or SAP232 with Ala, Cys, IIe, Val, or Phe substitutions are listed in Table 1 as examples.

In some instances, a Tyr on the hydrophobic side of a beta-strand can be replaced by a His as histidine residues have been reported to interact with tyrosine residues via the stacking of their aromatic rings and their ability to form hydrogen bonds [Seale 2006].

A multiplicity of amino acids of various charges, polarity or hydrophobicity can be substituted for His or Tyr residues in an alternating His-Tyr backbone peptide to modulate the properties of a self-aggregating peptide under various conditions of pH, salt and temperature.

It should be noted that while the SAPs comprising only alternating His and Tyr residues such as SAP119, SAP210, SAP231 or SAP232 are predicted to form beta-sheets at neutral pH, some of their derivatives may form alpha-helices or may aggregate or form other secondary structures that may be functionally useful and relevant for this invention. Ding et al. have shown that the EAK16 peptide forms beta-sheets when self-assembling on its own, but alpha-helices when fused to the HIV epitope SLYNTVATL [Ding 2016]. It is expected that because of their regular and repetitive sequences, many SAPs and the IFPs that comprise them, will form some secondary structures, beta-sheets, alpha-helices or other, leading to their insolubility. While not wishing to be bound by theory, the methods and designs disclosed herein begin with the assumption that some of the SAPs may form anti-parallel beta-sheets. However, it is agnostic to the actual secondary structure formed and only demands that these SAPs be insoluble and aggregate under neutral pH and be soluble under acidic or alkaline conditions.

While the description of this invention is illustrated by derivatives of prototypical self-aggregating peptides such as SAP119, SAP210, SAP231 or SAP232, it will be apparent to those skilled in the art that similar sequence variations of other self-assembling peptides can be designed to be pH-responsive and screened to have the appropriate insolubility at neutral pH and solubility at acidic conditions (less than pH 5) and/or basic conditions (pH greater than 9), so that they can, as part of a fusion protein and in conjunction with a process that manipulates pH, enable a low-cost purification of peptides, and thus not depart from the concept, spirit and scope of the invention.

### Design of New Insolubility Fusion Partners Composed of SAPs

New insolubility fusion partners (IFPs) in this invention have been designed to be insoluble at neutral pH while being possibly soluble at acidic or alkaline pH and to form anti-parallel beta sheets. To facilitate the formation of beta-sheets when an insolubility fusion partner is being synthesized by the cell, amino acid sequences were designed by “stringing” the sequences of one or more self-aggregating peptides that fulfill Criterion 1. Spacer sequences separating the SAPs will enable the proper anti-parallel orientation of one SAP relative to that of the previous one. Accordingly, it is an object of the invention to provide an IFP having the general structure:

[SAP]-[[Spacer]-[SAP]]m

wherein;
a) SAP is a self-aggregating peptide that fulfills Criterion 1;
b) the spacer is a peptide having from 1 to 50 amino acids;
c) m is an integer from 0 to 10;
   and wherein said peptide exhibits self-aggregating properties at neutral pH.

In some embodiments, the IFP will have a peptide length equal to or less than about 60 amino acids, e.g., 60 aa, 59 aa, 58 aa, 57 aa, 56 aa, 55 aa, 54 aa, 53 aa, 52 aa, 51 aa, 50 aa, 49 aa, 48 aa, 47 aa, 46 aa, 45 aa, 44 aa, 43 aa, 42 aa, 41 aa, 40 aa, 39 aa, 38 aa, 37 aa, 36 aa, 35 aa, 34 aa, 33 aa, 32 aa, 31 aa, 30 aa, 29 aa, 28 aa, 27 aa, 26 aa, 25 aa, 24 aa, 23 aa, 22 aa, 21 aa, 20 aa, 19 aa, 18 aa, 17 aa, 16 aa, 15 aa, or less. In preferred embodiments, the IFP will have a peptide length of about 45 amino acids or less, or about 30 amino acids or less, or about 15 amino acids or less. The SAPs in an IFP need not to be identical as long as they interact with each other to promote aggregation and insolubility. In the design of IFPs it is necessary to include a methionine as the initiating amino acid. In other embodiments, the amino acid sequence of the IFP may also include a short sequence at its N-terminus to maximize translation efficiency and increase expression such as Met-Ala-Ser. In some embodiments, spacers such as Pro-Arg-Gly or Pro-Glu-Gly are chosen as they are known to induce the formation of beta-turns. When two or more spacers are used, the sequences Pro-Arg-Gly and Pro-Glu-Gly can be used alternatively. In other embodiments, a Gly-rich flexible spacer such as Gly-Gly-Gly or Gly-Gly-Gly-Gly may be chosen.

In constructs where the IFP is expressed alone, the amino acid sequence may stop at the last amino acid of the last SAP segment. Alternatively, a C-terminal “cap” may be added such as a hexa-histidine (SEQ ID NO: 39) or a Pro rich oligo peptide such as Pro-Gly-Pro-Gly-Pro (SEQ ID NO: 40). In constructs where the IFP is fused to a peptide of interest (POI), a cleavable linker may be added. In a preferred embodiment, the linker with the sequence Gly-Gly-Asp-Pro-Gly-Gly (SEQ ID NO: 37) is added after the last amino acid of the last SAP segment, the Asp-Pro sequence being the well characterized acid labile cleavage site. In other embodiments, an additional Gly residue may be added to either or both termini of Gly-Gly-Asp-Pro-Gly-Gly (SEQ ID NO: 37). There are no limits to the number of SAPs included in the general formula, but less than 10 SAPs is preferred. In another variation, a sequence may be added at the C-terminus of the last SAP segment to include a linker that may have various functionalities, in particular to include an amino acid cleavage sequence or a charge modulating sequence.

In another embodiment, described herein is a set of IFPs where each IFP incorporates 3, 4 or 5 copies of self-aggregating peptides with sequences chosen among the SAPs listed in Table 1 (SEQ ID NO: 2 to SEQ ID NO: 35). For illustration, the IFP core structure is represented by [SAP]-spacer-[SAP]-spacer-[SAP]-spacer-[SAP]. The spacer may be a flexible sequence such as, but not limited to, Gly-Gly-Gly-Gly or a turn such as Pro-Arg-Gly (PRG) and Pro-Glu-Gly (PEG). In addition, other sequences may be fused to the core of the IFP, for example an initiating amino acid sequence such as Met-Ala-Ser or Met-Gly-Ser, a cleavage recognition site such as Asp-Pro, a C-terminal “cap” to protect the IFP expressed alone from proteolytic degradation such as multiple Gly-Pro and possibly a His6 sequence for purification and detection.

A prototypical IFP for this set incorporates four copies of SAP119. The amino acid sequence of its core can be derived from the structure SAP119-GGGG-SAP119-GGGG-SAP119-GGGG-SAP119. Alternatively, IFPs may be designed to include any number of copies of a chosen SAP, combinations of different SAPs or different spacers. They may also include N-terminal translation initiating sequences, cleavage sites and C-terminal caps. In preferred embodiments, the SAPs fulfilling Criterion 1 derive from peptides SAP119, SAP210, SAP231 and SAP232, and more generally, from other His-Tyr alternating peptides that fulfill Formula 1.

### Design of Fusion Peptides for the Production of Isolated POIs

To produce a peptide of interest (POI), fusion peptides can be designed by connecting an insolubility fusion partner (IFP), typically encompassing one or more SAPs, to a POI via a cleavable amino acid linker (cLNK). The connection can be at the N-terminus, at the C-terminus or at both termini of the POI (“split fusions”). Similarly, the IFP can be fused to one or multiple copies of the POI with each one separated by a cleavable linker. The fusion peptides are connected to the IFP by first chemically synthesizing the nucleic acid genetic construct using art-standard techniques and cloning the construct into an express plasmid for expression of the peptide in bacteria (see, e.g. Example 1). In general, synthesis methods for making oligonucleotides and nucleic acid constructs are well known in the art. For example, oligonucleotides can be synthesized chemically according to the solid phase phosphoramidite triester method described by Beaucage and Caruthers (1981) Tetrahedron Letts 22:1859-1862, e.g., using a commercially available automated synthesizer, e.g., as described in Needham-VanDevanter et al. (1984) Nucleic Acids Res. 12:6159-6168. Oligonucleotides, including modified oligonucleotides can also be ordered from a variety of commercial sources known to persons of skill. There are many commercial providers of oligo synthesis services, and thus this is a broadly accessible technology. Moreover, the technology for using microbial hosts to express constructs containing these synthetic oligonucleotides to produce the polypeptides of interest are also well known. Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”). More recently, DNA assembly and closing methods include exonuclease digestion ligation or PCR-based assembly as described in Gibson et al. [Nature Methods 6 (5): 343-345 (2009)] and TerMaat et al. [J. Microbiol. Methods 79 (3): 295-300 (2009)], respectively.

Variations of the fusion peptide are described by the formula:

[[SAP-CON-]m-POI-[CON-SAP]n-CON]p

wherein:
(a) p is an integer greater than 0;
(b) m is an integer including 0, wherein in any repeating segment represented by p, m can be the same integer or m can be a different integer from any other repeating segment represented by p;
(c) n is an integer including 0, wherein in any repeating segment represented by p, n can be the same integer or n can be a different integer from any other repeating segment represented by p;
(d) SAP is a self-aggregating peptide and may represent more than one self-aggregating peptide amino acid sequence;
(e) POI is a peptide of interest comprising more than one epitope amino acid sequence;
(f) CON is a connecting peptide and may represent more than one connector amino acid sequence;
   and wherein the last connector may be omitted.

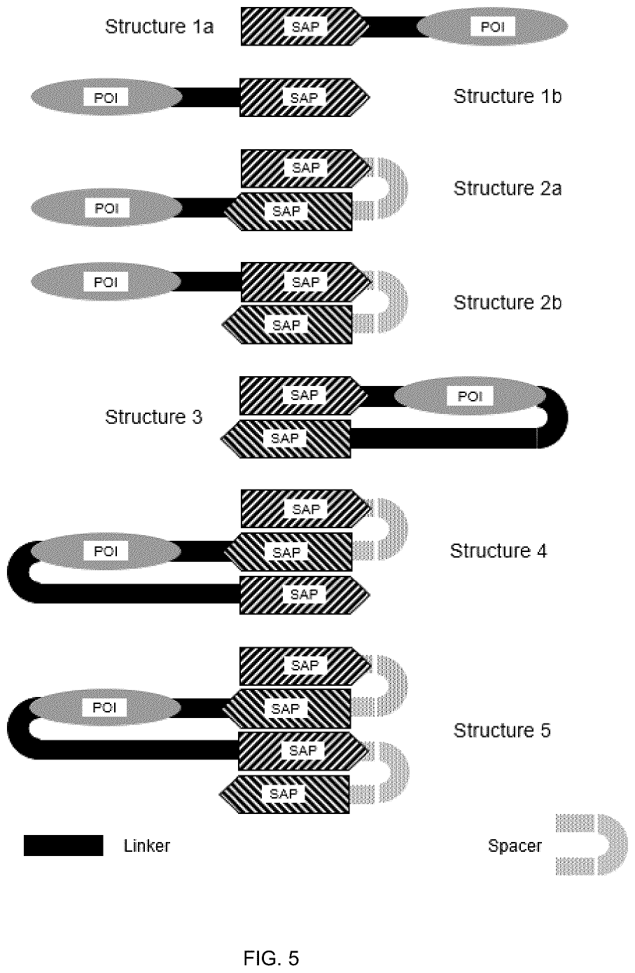

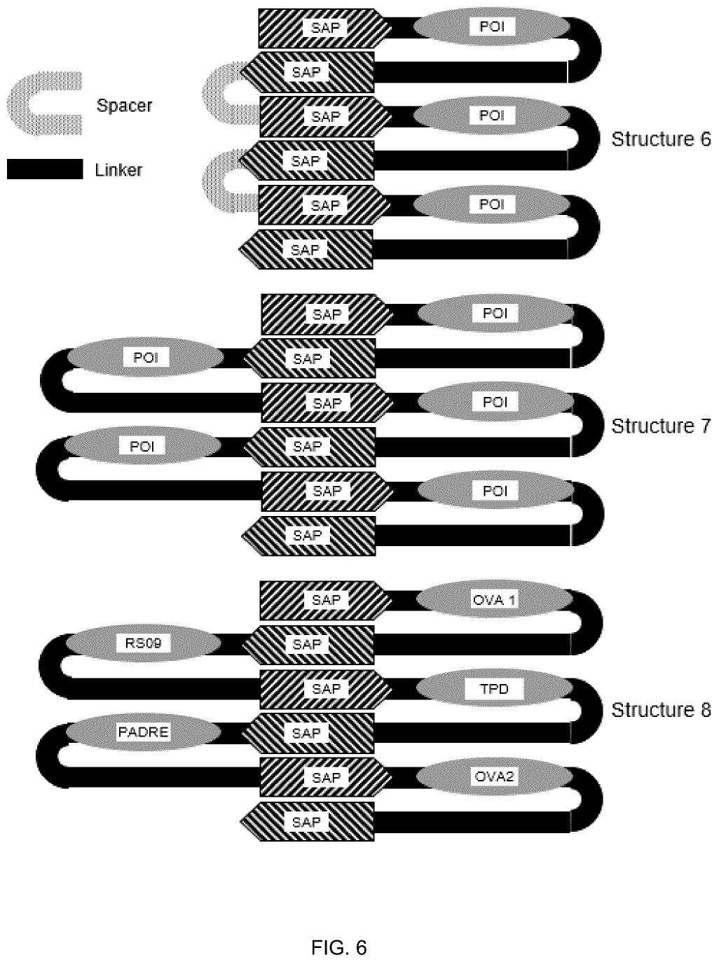

In some embodiments, m is an integer from 0 to 3, n is an integer from 0 to 3, and p is an integer from 1 to 8. In such embodiments, within any repeating segment represented by p, m and/or n can be the same integer or a different integer from any other repeating segment represented by p. In yet other embodiments, m is an integer from 0 to 2, n is an integer from 0 to 2, and p is an integer from 1 to 5 with m and/or n being the same integer or a different integer between any two repeating segments represented by p. For instance, in some structures:
m is 1, n is 0, and p is 1 (FIG. 5, structure 1a); or
m is 0, n is 1, and p is 1 (FIG. 5, structure 1b); or
m is 2, n is 0, and p is 1 (FIG. 5, structure 2a); or
m is 0, n is 2, and p is 1 (FIG. 5, structure 2b); or
m is 1, n is 1, and p is 1 (FIG. 5, structure 3); or
m is 2, n is 1, and p is 1 (FIG. 5, structure 4); or
m is 2, n is 2, and p is 1 (FIG. 5, structure 5); or
m is 1, n is 1, and p is 3 (FIG. 6, structure 6); or
m is 1, n is 0, and p is 5 (FIG. 6, structure 7).

Various fusion architectures covered by the formula [[SAP-CON-]m-POI-[CON-SAP]n-CON]p can be envisioned and include, although not limited to:

IFP-cLNK-POI;

POI-cLNK-IFP;

IFP-[cLNK-POI]n;

[POI-cLNK]n-IFP;

IFP-cLNK-POI-cLNK-IFP;

IFP-[cLNK-POI-cLNK-IFP]n;

wherein IFP represents one of multiple different insolubility fusion partners, POI represents one or multiple different peptides of interest, cLNK represents one or multiple different cleavable linkers and n is a number greater than 1.

The production of “simple fusions” with the architecture IFP-cLNK-POI and IFP-cLNK-POI-cLNK-POI is presented in Example 4. The production of “split fusions” with the architecture IFP-cLINK-POI-cLNK-IFP and IFP-cLNK-POI-cLNK-IFP-cLNK-POI-cLNK-IFP is presented in Example 5

For demonstration purposes, several POIs can be chosen from the literature by having demonstrated benefits for human health while not being commercialized due in part to their prohibitive cost of manufacturing. Among them can be: LL37, a 37-aa antimicrobial peptide derived from a human cathelicidin with numerous biological activities [Xhindoli 2016], Lunasin, a 43-aa soy-derived peptide reported to have many health benefits [Fernandez-Tome 2019], and Apidaecin, an 18-aa antimicrobial peptide [Torres 2019] or Tachyplesin, a 17-aa antimicrobial peptide [U.S. Pat. No. 11,352,396].

It is understood that other medically or commercially relevant peptides can be used as POIs for proof-of-concept demonstration and would constitute a reduction to practice falling under the spirit of the invention.

### Genetic Engineering of Host for the Production of Peptides

The genetic engineering of microbial hosts for the production of proteins has become routine thanks to the commercial availability of gene design, synthesis, and cloning, and to expression services provided by commercial companies. It well within the purview of those skilled in the art to design the amino acid sequence of a polypeptide of interest and obtain the chemically synthesized plasmid, e.g., from a molecular biology company, that codes for the polypeptide of interest and drives its expression in a chosen microbial host. As such, one having ordinary skill in the art would readily understand the disclosure herein with regard to providing a genetically engineered microbial host or genetically engineering a microbial host to express a nucleic acid construct that codes for the peptide based on the designs discussed above and enables the microbial host to express such peptide when subjected to suitable medium as is well understood in the art.

Typically, those skilled in the art can request from molecular biology service companies (such as Twist Bioscience, Thermo-Fisher/Invitrogen/GeneArt or GeneScript) the specific design elements for genes encoding the polypeptide of interest such as a codon usage specifically adapted to the chosen microbial host, the desired expression level by selecting the most abundant codons, the absence or presence of predicted secondary mRNA structures, the presence of specific nucleotide sequences encoding efficient translation initiation, the choice of specific promoters and regulatory sequences determining an appropriate expression level and inducibility or repression. Those skilled in the art can also request that the genes synthesized be cloned in an expression plasmid vector of choice, with a specific copy number, in a host that encodes appropriate regulatory elements to repress or induce RNA synthesis from the chosen promoter. They can also further request that the designed expression plasmid encoding the gene of the polypeptide be introduced into the expression host cells.

In the current state of molecular biology services, the specific gene sequence encoding a specific peptide is no longer critical since gene synthesis companies offer various optimization protocols to maximize gene expression. Alternate gene sequences, alternate engineered hosts, alternate plasmid vectors, alternate promoter systems and regulatory elements, alternate polypeptide coding sequences can be used interchangeably and still lead to the production of the desired peptide with the desired amino acid sequence in the spirit of this invention.

Examples of promoters used for the expression of proteins in E. coli include the lac promoter and its derivatives that can be repressed by glucose and be induced by lactose as well as by the non-natural inducer IPTG (isopropyl-β-D-thiogalactoside), the arabinose promoter that can be induced by L-arabinose and is repressed by glucose, the T7 promoter that is recognized by the phage T7-RNA polymerase that is provided by the host cell.

Examples of microbial host strains include, but are not limited to, fungal or yeast genera such as Aspergillus, Trichoderma, Saccharomyces, Pichia, Candida, Hansenula, Yarrowia, and bacterial genera such as Salmonella, Bacillus, Lactobacillus, Lactococcus, Clostridium, Staphylococcus, Streptococcus, Acinetobacter, Corynebacterium, Zymomonas, Agrobacterium, Chlorobium, Rhodobacter, Rhodococcus, Streptomyces, Deinococcus, Escherichia, Pseudomonas, Methylomonas, Methylobacter, Alcaligenes, Ralstonia, Synechocystis, Synechococcus, Anabaena. Preferred expression hosts that are commercially used include Aspergillus, Trichoderma, Saccharomyces, Pichia, Escherichia, Bacillus, Pseudomonas, Lactobacillus, Lactococcus, Corynebacterium and Clostridium. In a more preferred embodiment, the host strain is Escherichia coli.

Recently with the advent of the CRISPR technology [see, for example, Zhang et al., Front Bioeng. Biotechnol. 7:459 (2020)], it has also become much easier to engineer an expression system in a selected host where one or more copies of the gene encoding the polypeptide along with its selected promoter and expression system are integrated in the chromosome of the host if a plasmid-free production is needed.

### Biological Production of Peptides

The invention provides methods for the production and recovery of fusion peptides comprising combinations of SAPs, IFPs and POIs, all designed with pH specific solubilities to facilitate and simplify purification and recovery. In general, the production method of the invention will include the following steps:
a) providing a genetically engineered microbial host cell that expresses a genetic construct encoding the amino acid sequence of a fusion peptide, wherein said fusion peptide comprises:
   i) an insolubility fusion partner that is insoluble at neutral pH and soluble at acid pH;
   ii) a linker comprising a cleavage site; and
   iii) a peptide of interest that is soluble at neutral pH;
b) growing the microbial host under conditions wherein said fusion peptide is produced in an insoluble form in the host cytoplasm;
c) recovering the insoluble fusion peptide of step b);
d) solubilizing the recovered insoluble fusion peptide of step c) in an acidic medium;
e) cleaving the insolubility fusion partner from the peptide of interest; and
h) altering the pH to precipitate the insoluble fusion partner and recovering the peptide of interest in soluble form.

The implementation of the method of the invention therefore entails engineering genetically a microbial host to include a nucleic acid segment coding for a fusion peptide comprising an insoluble fusion partner linked to a peptide of interest by a peptide linker wherein the fusion peptide is designed to be insoluble at neutral pH and/or soluble at acidic or alkaline pH. This will be followed by growing the genetically engineered host cell under conditions where the encoded fusion peptide is produced in an insoluble form in the cytoplasm of the host cell. At this point the insoluble fusion peptide is recovered after a physical separation from the soluble host cell components. The fusion peptide is then subjected to one or more treatments in which an aqueous medium having a pH at which said IFP becomes soluble. The solubilized fusion peptide is then recovered in the aqueous phase after a physical separation of the insoluble host cell components and the composition of the solution containing the solubilized fusion peptide is adjusted to be appropriate for an effective cleavage treatment. At this stage, the cleavable linker of the fusion peptide is cleaved to separate the IFP from the POI and possibly several POIs among themselves. Finally the composition of the solution containing the peptide of interest is adjusted to a neutral pH at which the IFP becomes insoluble and the POI is recovered in the aqueous solution after physical separation of the insolubility fusion partner.

Those skilled in the art will find numerous references in the literature for growth medium and cultivation protocols optimized for the recombinant production of proteins, whether in shake flask cultures or fermentor cultures. The growth of peptide-expressing hosts is carried out in a fermentation medium appropriate both for the maximum growth of the cells of the specific host, and for a maximal peptide titer. Numerous media formulations and fermentation protocols can be found in the literature for each specific host and can be optimized by those skilled in the art. Critical are the carbon source of the medium, for example sugars for E. coli, many Bacillus sp. or Saccharomyces, organic acids or amino acids for Pseudomonas, methanol for Pichia; the nitrogen source, for example ammonium, urea or amino acids; as well as an appropriate supply of phosphorus and sulfur. Salts should also be added to provide sodium, potassium, sulfur and phosphate ions as well to control the osmolarity optimal for the physiology of the host cell. Many microbes also need trace minerals and vitamins. Finally, under protein production fermentation conditions, the biosynthetic burden on the cell can be minimized by providing all the nucleotides, amino acids and other cell building blocks. This can be done by adding complex components to the medium such as yeast extract or animal or plant hydrolysates. For the production of specific peptides with a biased amino acid composition, it may be advantageous to provide additional amounts of the over-represented amino acids. Control of the optimal pH in the fermentation can be carried out by the appropriate buffer such as phosphate, CO2/bicarbonate or synthetic buffers like Tris.

Protocols for the maximum production of proteins may vary with the host as well as the specific protein to be produced. In many cases it is beneficial to induce the production of protein after a growth phase that builds biomass while in other cases it is beneficial to produce a protein throughout the growth phase. Those skilled in the art will find numerous references in the literature for the induction of protein production. Such protocols vary according to the promoter used. For example, the lac (lactose) promoter of E. coli and its derivatives can be induced by the addition of lactose or IPTG, the arabinose promoter can be induced by L-arabinose, the cellobiose hydrolase promoter of Trichoderma can be induced by cellobiose, the aldehyde oxidase promoter of Pichia can be induced by methanol while the galactokinase promoter of Saccharomyces can be induced by galactose. Some proteins are better expressed by strong constitutive promoters while others are by growth-rate regulated promoters. Finally some expression systems rely on the disappearance of molecules repressing expression. A well-characterized repression in several organisms is the so-called catabolite repression caused by glucose. In E. coli for example, inducing carbohydrate substrates cannot induce transcription from their cognate promoters as long as glucose is present. This can be advantageous to maximize the cell density in the culture and delay the induction of the protein. An example of a medium that makes use of this system is the auto-induction medium (per L: 10 g/L Tryptone, 5 g/L Yeast Extract, 5 g/L NaCl, 50 mM Na2HPO4, 50 mM KH2PO4, 25 mM (NH4) 2SO4, 3 mM MgSO4, 0.75% glycerol, 0.075% glucose and 0.05% L-arabinose) [Studier 2005].

At the end of the growth phase, cells expressing SAPs or IFPs alone or fused to a POI will show the cytoplasmic accumulation of the expected polypeptides using denaturing polyacrylamide gel electrophoresis (SDS-PAGE). To assess if the polypeptide accumulates in an insoluble form, as well to produce the peptide of interest, cells must be disrupted. The solubility properties of an SAP are determined and defined by the solubility properties of the IFPs that comprises it.

### Recovery of Inclusion Bodies

Disruption of host cells to release their cytoplasmic content can be performed mechanically, enzymatically, chemically or biologically. Following resuspension of the cells in lysis buffer (e.g. 50 mM Tris pH 7.5, 10 mM EDTA, 50 mg/L lysozyme), mechanical disruption can be achieved by grinding or beating a cell suspension with a hard material such as silica, titania or zirconia beads, by a pressure drop in an apparatus like a French press or a Gaulin homogenizer, or by sonication. Enzymatic disruption can be performed by treatment with enzymes that hydrolyze the cell wall of the microbial host such as lysozyme for bacteria or Zymolyase® (Zymo Research, Irvine CA) for fungal hosts. Chemical disruption may be performed by cocktails of detergents, chaotropes and extreme pH. Biological disruption can be achieved by the activity of a biological agent that triggers the host cell to lyse such as an endolysin, a bacterial phage or a fungal virus. Combinations of enzymatic and chemical treatments are particularly useful at small scale for the rapid screening of multiple cultures and are available commercially (e.g. CelLytic® from Sigma-Aldrich). At larger scale, for research and production purpose, mechanical disruption methods are preferred.

Following the disruption of cells, the cell extracts are separated into soluble and insoluble fractions by physical methods such as centrifugation, sedimentation or filtration. The soluble fraction contains small molecules, soluble macromolecules like sheared nucleic acids, ribosomes and soluble proteins. The insoluble fraction contains mostly cell wall and membrane debris, insoluble proteins and overexpressed proteins in the form of inclusion bodies. The inclusion bodies themselves often contain entrapped macromolecules that need to be further removed.

Following centrifugation, the soluble fraction is discarded and the pellet is washed several times by successive resuspensions and centrifugations. The resuspension solution may comprise a combination of buffer, salt, detergent, multivalent ions, enzyme and enzyme inhibitors. After the last centrifugation, the overexpressed polypeptides may represent more than 50% of the proteins, but are still contaminated by cell wall and membrane debris.

Selection of IFPs and SAPs with Desired Solubility Properties

To enable a physical separation of the inclusion bodies from remaining particulate host-cell contaminants, the strategy described in this invention is to solubilize the inclusion bodies by a low pH treatment. This step relies on the solubility properties of the SAPs that are engineered in the sequence of the IFP in particular by the incorporation of charged amino acids as described above.

IFPs with the best properties can be screened after overexpression in E. coli. Following growth and induction of their expression at a small scale, e.g. 1-20 mL cultures, cells are lysed with a commercial lysis cocktail such as the CelLytic® extraction reagent from Sigma-Aldrich that contains a nuclease, a lysozyme and detergents. This treatment solubilizes most of the cell wall and the membranes, leaving inclusion bodies essentially as the sole particulate fraction. The particulate fraction can then be separated from the cell wall and membranes using physical separation techniques known in the art, such as, but not limited to centrifugation, filtration, sedimentation, or some combination thereof. The presence and relative abundance of inclusion bodies can be assessed by the remaining turbidity after treatment. Extracts that retain the greatest turbidity after lysis correspond to IFPs produced in greatest amount. These inclusion bodies can then be washed in water or in a buffer at neutral pH as described above, and resuspended in buffers of various pH to evaluate their solubility. The pH typically range from pH 5 to pH 3 for acidic conditions; and range from pH 9 to pH 13 for alkaline conditions. This characterization leads to the selection of the IFPs with the most desirable solubility properties described above. In a preferred embodiment, the IFPs are insoluble between pH 6 and 8, soluble at a pH below 5, and soluble at a pH above 11. It is expected that the properties of these “best” IFPs correspond to the solubility properties of the SAPs they comprise.

The same method can also be used to assess the solubility properties of a peptide of interest expressed as a fusion to SAPs or IFPs under various architectures and to allow the selection of the best SAP or IFP to match the purification process described below. Also, as the POI adds its own solubility properties to that of the IFP in a fusion peptide, a set of IFPs with different solubility properties is useful to match a POI with the IFP that will result in the most efficient production of purified POI.

### Purification of the Fusion Peptide at Acid pH

The purpose of a low pH treatment is to remove additional host-cell contaminants that are insoluble after cell disruption and are recovered with the inclusion bodies after physical separation, e.g., centrifugation. This purification step hinges on the solubilization of the fusion peptide at acidic pH. Once a fusion design has been selected to produce a specific peptide of interest, it can be tested in a laboratory-scale process that is predictive of a commercial-scale process. In a preferred embodiment, the method for producing and recovering the peptides of this invention will include this purification step as a core step in the process.

After growth and induction of cells expressing a peptide of interest fused to an IFP or an SAP selected for insolubility at neutral pH and solubility at acidic pH, cells are harvested, washed and disrupted by a physical process such as bead-beating, French pressure cell, or sonication. The insoluble fraction containing the inclusion bodies and insoluble host-cell contaminants are recovered by physical separation, such as, but not limited to centrifugation. Insoluble host-cell contaminants include cell wall and membrane debris, insoluble host proteins and possibly soluble host proteins that may be entrapped in the inclusion bodies.

The insoluble fraction is resuspended in a solution the pH of which is lowered by addition of acid such as, but not limited to, hydrochloric acid, formic acid, acetic acid or phosphoric acid. The pH value selected can range between 5 and 2, based on the solubility properties of the SAP and IFP selected for the fusion to the POI. At low pH, many of the cell debris such as cell walls and membranes are expected to remain insoluble while the inclusion bodies are expected to solubilize. Furthermore, in doing so, soluble host proteins that had been carried over so far are expected to be released from the inclusion bodies and, like the majority of the E. coli proteins, to precipitate at acidic pH due to the exposure of their hydrophobic cores that are normally buried. At pH<3, the majority of globular proteins have a maximal positive charge that in most cases leads to their denaturation and irreversible precipitation. Thus, the solubilized fusion proteins can be separated from the host-cell contaminants that remain insoluble.

### Purification of the Fusion Peptide at Alkaline pH

In some instances it may be beneficial to remove additional contaminating proteins that remain soluble at acidic pH, which is the case for basic proteins such as ribosomal proteins. This can be achieved by using an alkaline treatment. As described above, the solubility characterization of various SAP and IFPs can identify those that become insoluble at alkaline pH. At pH greater than 12, the majority of alkaline proteins have a maximal negative charge that in most cases leads to their denaturation and irreversible precipitation. As in the case of acidic pH purification, it is anticipated that additional proteins will precipitate while selected IFPs will remain in solution, thus enabling their purification.

Cleavage of Peptide of Interest from Insolubility Fusion Partner

After its expression as part of a fusion peptide and after acidic and optionally alkaline pH purification, the peptide of interest (POI) must be cleaved from its IFP at a cleavage sequence in the linker. This can be achieved enzymatically using sequence specific protease (e.g. Factor Xa, TEV protease, enterokinase, thrombin or inteins) [LaVallie 1994]. However, most of these enzymes have low activity, are expensive and difficult to scale up. The cleavage can also take place with a chemical reagent such as cyanogen bromide or hydroxylamine, both of which are highly toxic and difficult to use at large commercial scale.

In a preferred embodiment, the separation of the POI from the IFP is carried out by acid cleavage of the peptide bond between the Asp-Pro amino acids (DP) engineered in the linker separating the POI from the IFP or between multiple copies of the POI, for example in 50% formic acid at 70° C. for 24 hrs [LaVallie 1994]. At the completion of the cleavage in acid, both the POI and the IFP are expected to be soluble.

Recovery of the Cleaved POI from the IFP

Once the POI is cleaved off from the IFP, it can be recovered by a variety of methods that include physical methods such as filtration, precipitation with solvents or salts like ethanol or ammonium sulfate, and chromatographic methods such as ion exchange, hydrophobic interaction, reverse phase and affinity chromatography or by lyophilization.

In a preferred embodiment, after acid cleavage, the soluble POI can be separated from the IFP by adjusting the pH of the cleavage solution back to neutrality. Under these conditions, the insolubility fusion partner becomes insoluble, as designed, and can be separated physically from the neutralized solution containing the soluble peptide of interest.

It is known to those skilled in the art that adjusting the temperature, the salt composition and concentration, or some other chemical agents may enhance further the difference of solubility between the IFP and the POI and improve this purification step.

Design of Insoluble Peptides with Bioactive or Immunogenic Functionalities

In many applications, it can be useful to link physically the self-assembly or aggregation properties of a self-aggregating peptide (SAP) with the functionality of the peptide of interest (POI). This is in particular the case for the development of biomaterials displaying a biological function.

Of particular interest are multi-functional peptides that comprise one or more self-aggregating peptides linked with one or more peptide epitopes (i.e. POI) in order to convey bioactivity or immunogenic functionality. In a general application, the epitope will comprise an amino acid sequence that is recognized by a cellular receptor resulting in a physiological response. In some instances the epitope will function as an immunogen, being recognized by an immune system, by antibodies, antigen-displaying cells, B cells or T cells; and it will have an immunogenic functionality. Alternatively in other applications, the epitope may also be recognized by other receptors such as a hormonal receptor, a metabolite receptor, a taste receptor, sensory receptor, or by other cell-surface receptors for targeting cells such as cancer cells.

Several examples of peptide architectures that include one or more self-aggregating peptides and one or more immunogenic peptides to be displayed were designed:
Structure 1: SAP-LNK-POI or POI-LNK-SAP;
Structure 2: SAP-Spacer-SAP-LNK-POI or POI-LNK-SAP-Spacer-SAP
Structure 3: SAP-LNK-POI-LNK-SAP
Structure 4: SAP-Spacer-SAP-LNK-POI-LNK-SAP
Structure 5: SAP-Spacer-SAP-LNK-POI-LNK-SAP-Spacer-SAP.
   Schematic representations of these structures are presented in FIG. 5.

In Structures 1a and 1b (SAP-LNK-POI or POI-LNK-SAP), the immunogenic epitope (POI) is linked at its N-terminus or its C-terminus to a single SAP to enable the bioproduction of a single epitope POI that can be displayed at the surface of aggregates with various structures. The linker may be of variable length to modulate the accessibility of the epitope.

In Structures 2a and 2b (SAP-Spacer-SAP-LNK-POI and POI-LNK-SAP-Spacer-SAP), the immunogenic epitope is linked at its N-terminus or its C-terminus to two SAPs. The presence of two self-aggregating peptide domains is expected to enhance the insolubility of the overall peptide and improve the bioproduction and purification process. The linker may be of variable length to modulate the accessibility of the epitope.

In Structure 3 (SAP-LNK-POI-LNK-SAP), Structure 4 (SAP-Spacer-SAP-LNK-POI-LNK-SAP) and Structure 5 (SAP-Spacer-SAP-LNK-POI-LNK-SAP-Spacer-SAP), the immunogenic epitope is sandwiched between SAPs at its N-terminus and its C-terminus. It is expected that this configuration, in which the ends of the POI are not free, can result in greater protection of the epitope from proteolysis in production and in application. In addition, Structure 3 displays the POI epitope in a constrained form to mimic the structural environment in the original protein antigen and thus provide more effective vaccines. Examples of such prototypical insoluble immunogenic peptide designs are represented in FIG. 5.

It is understood that additional structures that comprise more copies of POIs, of linkers, of spacers or of SAPs can be designed. FIG. 6 represents such designs. In Structure 6, three copies of a POI are interspersed with six SAPs. In Structure 7, five copies of a POI are interspersed with six SAPs. FIG. 6 illustrates how the design of the IIPs may be used to display epitopes differently. The linkers and spacers may be of variable length to modulate the accessibility of the epitope. There is no limit to the number of SAPs, POIs, linkers and spacers that can be used in the design of an IIP. The SAPs, POIs, linkers and spacers do not have to be identical. Structure 8 depicts an IIP displaying 5 different POI epitopes. Examples of such prototypical insoluble immunogenic peptide designs are represented in FIG. 6, and the production of these peptides is summarized in Example 9.

### Design of Catenated Peptides for the Bioproduction of Single-Epitope Insoluble Immunogenic Peptides

For some applications, it may be advantageous to separate the short individual insoluble immunogenic peptides, such as those described by Structures 1, 2, 3, 4 or 5 (see FIG. 5), while their production may benefit from their expression in the form of longer catenated peptides (see FIG. 6).

To separate individual IIPs, some of the linkers or some of the spacers are chosen to include a cleavable sequence specific to the cleavage method, be it enzymatic or chemical. As a demonstration, a catenated version of Structure 3 in FIG. 5 (SAP-LINK-POI-LINK-SAP) can be modified to have Structure 6 as shown in FIG. 6, in which multiple repeats of SAP-LNK-POI-LNK-SAP are stringed and separated by a cleavable spacer abbreviated cSpacer.

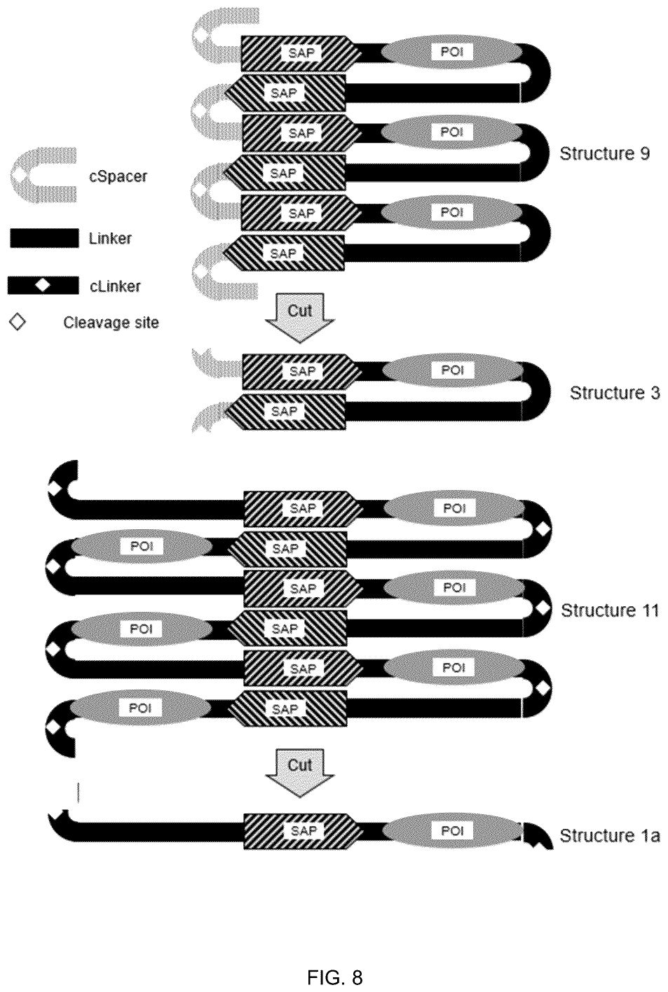

To ensure the homogeneity of the peptides produced after cleavage, the sequence of a cleavable spacer is added to the N-terminus and the C-terminus of the catenated sequence. After cleavage, the peptides that are released have the Structure 3, in which the epitope peptide is sandwiched between two SAPs. The structure of the catenated insoluble immunogenic peptide of Structure 9 includes three copies of an IIP similar to that of Structure 3 (see FIG. 8), and the production of this peptide is described in Example 10.

To illustrate, a catenated version of Structure 1a (see FIG. 5; SAP-LINK-POI) in which the epitope peptide was linked to a single self-aggregating peptide at its N-terminus, was designed to have the architecture represented in Structure 7 (see FIG. 6). In this architecture, multiple repeats of SAP-LNK-POI were stringed and separated by a cleavable linker (abbreviated cLNK). The sequence of the non-cleavable linker is GGSGGSGG (SEQ ID NO: 36) and the sequence of the cleavable linker cLNK is GGSGDPGSGG (SEQ ID NO: 38). The POI epitope chosen was OVA1. To ensure the homogeneity of the peptides produced after cleavage, the sequence of a cleavable linker or part of it was added to the N-terminus and the C-terminus of Structure 1a. The structure of the catenated IIP that comprises six copies of an IIP of structure 1a is represented as Structure 11 (see FIG. 8).

In preferred embodiments, the SAPs used have improved solubility properties that can be controlled by modulating the pH of the solution (insoluble at neutral pH, high expression level in host, soluble at acidic and/or alkaline pH) such as some of the SAPs fulfilling Criterion 1. In a more preferred embodiment, the SAP fulfilling Criterion 1 derives from a peptide following Formula 1 (SEQ ID NO: 1) in which His and Tyr residues are located on opposite sides of a predicted beta-sheet. In another more preferred embodiment, the SAP fulfilling Criterion 1 is predicted to form an amphiphilic alpha-helix with in which the Tyr residues are on one face of a 7-fold periodicity helix.

The immunogenic designs presented here are for example only and their architectures are expected to be applicable to a large range of constrained peptides. By stringing self-aggregating modules, linkers, and immunogenic epitopes in various numbers and combinations, different immunogenic structures can be produced. Such structures can combine (1) the properties of high density of epitopes displayed in a supra-molecular structure such as fibrils or films, (2) the formation of high or low molecular weight aggregates, (3) the efficient production in a microbial host and (4) an efficient purification in a process based on pH manipulation for efficacy and economic production.

The utility of integrating SAPs with pH-controlled solubility in the final structure of the product to be manufactured are: (1) the high yield expression of the peptides in an insoluble form inside a microbial host, (2) the lower cost of purification of the peptide product from host contaminating molecules, (3) the formation of the product structure and (4) the expected improved immunogenicity of the peptide via the display of multiple epitopes, in particular in the case of peptide-based biomaterials or vaccine nanoparticles.

The production and recovery of a bioactive fusion peptide of the invention wherein the POI of the fusion peptide comprises an immunogenic or bioactive epitope may begin by genetically engineering a microbial host to include a nucleic acid segment coding for an immunogenic or bioactive peptide that is insoluble at neutral pH and soluble under acidic conditions. This immunogenic or bioactive peptide will preferably have an architecture as described above. The next steps may include growing the genetically engineered host cell under conditions where the genetic construct is expressed and the encoded immunogenic or bioactive peptide is produced in an insoluble form in the cytoplasm of the host cell; recovering the insoluble peptide after physical separation from soluble host cell components; subjecting the insoluble immunogenic or bioactive peptide to an aqueous medium having a pH at which said insoluble immunogenic or bioactive peptide becomes soluble; recovering the solubilized immunogenic or bioactive peptide in the aqueous phase after physical separation of insoluble host cell components; adjusting the composition of the solution containing the immunogenic or bioactive peptide to a neutral pH at which it becomes insoluble; and recovering the insoluble immunogenic or bioactive fusion peptide after physical separation.

### EXAMPLES

The present invention is further defined in the following Examples. It should be understood that these Examples, while indicating preferred embodiments of the invention, are given by way of illustration only. From the detailed description of the invention and these Examples, one skilled in the art can ascertain the essential characteristics of this invention and, without departing from the spirit and scope thereof, can make various changes and modifications of the invention to adapt it to various uses and conditions.

### General Methods

The meaning of abbreviations used is as follows: “min” means minute(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmol” means micromole(s), “pmol” means picomole(s), “g” means gram(s), “μg” means microgram(s), “mg” means milligram(s), “g” means the gravitation constant, “rpm” means revolutions per minute and “cat #” means catalog number.

Cloning and protein expression technologies that may be used in the following examples are described in technical literature of suppliers of molecular biology services as follows:
Thermo-Fisher (2015). Protein expression handbook: Recombinant protein expression and purification technologies; Thermo-Fisher (2015) GeneArt Gene to Protein Handbook; Thermo-Fisher (2020) GeneArt Gene Synthesis; Thermo-Fisher (2015) GeneArt Gene Synthesis and services.

### Example 1

### Cloning and Expression of Insolubility Fusion Partners, Fusion Peptides and Insoluble Immunogenic Peptides

This example describes the procurement of E. coli strains that expressed peptides with sequences engineered for specific functionality.

The amino acid sequences of new IFPs were derived from the formula MAS-SAP-GGGG-SAP-GGGG-SAP-GGGG-SAP wherein MAS represents the initiating tripeptide Met-Ala-Ser, GGGG represents the Gly-Gly-Gly-Gly spacer and SAP represent a self-aggregating peptide fulfilling Criterion 1. The amino acid sequence of fusion peptides and IIPs that incorporated various SAPs, connectors and POIs could be derived from peptides structures such as those described in Tables 3 and 4. In some constructs, additional sequences were added at the C-terminal of the IFP, the fusions or the IIPs such as a Hexa-histidine (His6, SEQ ID NO: 39) or a proline-rich cap like PGPGP (SEQ ID NO: 40). Two stop codons marked the end of the gene.

To clone and express the genes for IFPs, fusion peptides or IIPs, chemically synthesized DNA constructs based on the designed amino acid sequences were carried out by a molecular biology technology provider (Twist Bioscience, South San Francisco, CA). Nucleic acid constructs based on the designed peptide sequence were synthesized by the provider to maximize expression using codon optimization for E. coli, to minimize secondary structures, to provide a 3′-end terminal stem loops to increase mRNA stability, to avoid restriction sites, to avoid repeats in the nucleotide sequence, etc. The synthesized genes were cloned by Twist Bioscience in the Ndel-Xhol restriction sites of the T7 expression plasmid pET-29b(+) with the initial Met of the peptides corresponding to the ATG of the Ndel restriction site of pET-29b(+) (SEQ ID NO: 116). To clone the genes encoding smaller peptides, it was necessary for Twist Bioscience to add additional “stuffer” DNA at the 3′-end of the gene. Ten ng of the plasmids carrying the cloned genes were transferred into chemically competent E. coli strain BL21-AI (BL21-AI™-One-Shot™, Invitrogen Waltham, MA) as described by the manufacturer. Fifty μL of the transformation culture were plated onto Luria-Bertani agar plates containing 30 μg/mL of kanamycin and 1% glucose (Teknova Hollister, CA) and grown overnight at 37° C. Strain BL21-AI carries a chromosomal insertion of a cassette containing the T7 RNA polymerase (T7 RNAP) gene in the araB locus, allowing expression of T7 RNAP to be regulated by the araBAD promoter and be repressed by glucose. Genes expressed in pET-29b (+) are under the control of both the T7 promoter and the Lacl repressor and are induced by the simultaneous presence of L-arabinose and IPTG.

To express the peptides, 5 to 10 colonies from the transformation plates were inoculated in 3 mL of auto-induction medium (per L: 10 g/L Tryptone, 5 g/L Yeast Extract, 5 g/L NaCl, 50 mM Na2HPO4, 50 mM KH2PO4, 25 mM (NH4) 2SO4, 3 mM MgSO4, 0.75% glycerol, 0.075% glucose and 0.05% L-arabinose) containing 30 μg/mL of ampicillin and 1 mM IPTG. The cultures were grown in 50 mL conical tubes with loosely fit screw cap tubes placed in a Cel-Gro™ tissue culture rotator (Thermo Scientific, Waltham, MA) at 37° C. for 18 to 24 hrs. Aliquots of each culture were taken and the production of the peptide assessed for each strain.

This protocol was used to express IFPs alone, IFP fusions to peptides of interest (IFP-POI) and Insoluble Immunogenic Peptides (IIPs).

### Example 2

### Demonstration of Insolubility of New Fusion Partners

This example describes a general protocol to assess the solubility of polypeptides expressed in E. coli. It also provide a method to evaluate the self-aggregating properties of Self Aggregating Peptides (SAPs) in the context of biologically produced Insolubility Fusion Partners (IFPs) that include multiple copies of an SAP.

As described above, numerous IFPs were designed according to the structural guidance provided herein. In particular, the IFPs include the following characteristics:
(a) the initiating tripeptide Met-Ala-Ser
(b) a Gly rich linker such as Gly-Gly or Gly-Gly-Gly
(c) a core with the structure [SAP]-(spacer-[SAP]) n in which:
   SAP is a self-aggregating peptide fulfilling formula 1
   LNK is a linker such as GGGG or PRG or PEG
   n is 2-4
(d) a flexible linker such as Gly-Gly-Gly, Gly-Gly-Gly-Gly; and
(e) an optional C-terminal cap, such as PGPGP (SEQ ID NO: 40) and/or HHHHHH (SEQ ID NO: 39).
   A list of representative IFPs is shown in Table 4. For example, the sequence of IFP534 is: MAS-GGGG-QQHYHYHQQ-GGGG-QQHYHYHQQ-GGGG-QQHYHYHQQ-GGGG-QQHYHYHQQ -GGGG-PGPGP (amino acid SEQ ID NO: 62) and encoded by nucleic acid SEQ ID NO:63, and that of IFP221_PRG is:
   MAS-GGG-QQEYHYHYHYRQQ-PRG-QQEYHYHYHYRQQ-PEG-QQEYHYHYHYRQQ-GGGG-PG PGP (amino acid SEQ ID NO:50) and encoded by nucleic acid SEQ ID NO:51.

TABLE 4
Representative Insolubility Fusions
Partners Expressed in E. coli

| | | | No. | |
|---|---|---|---|---|
| IFP | SAP | | of | |
| (SEQ ID NO) | Sequence | SEQ ID NO: | SAPs | Linker |
| IFP221_PRG | QQEYHYH | SEQ ID NO: 11 | 3 | PRG |
| (SEQ ID NO: 50) | YHYRQQ | | | and |
| | | | | PEG |
| IFP365 | QQHFHFH | SEQ ID NO: 30 | 4 | GGGG |
| (SEQ ID NO: 52) | FHQQ | | | |
| IFP380 | QQHYHYH | SEQ ID NO: 9 | 4 | GGGG |
| (SEQ ID NO: 54) | YHQQ | | | |
| IFP381 | QQEYHYH | SEQ ID NO: 10 | 4 | GGGG |
| (SEQ ID NO: 56) | YRQQ | | | |
| IFP462 | HQEYHYH | SEQ ID NO: 12 | 4 | GGGG |
| (SEQ ID NO: 58) | YRQH | | | |
| IFP463 | HQEYHVH | SEQ ID NO: 28 | 4 | GGGG |
| (SEQ ID NO: 60) | YRQH | | | |
| IFP534 | QQHYHYH | SEQ ID NO: 6 | 4 | GGGG |
| (SEQ ID NO: 62) | QQ | | | |
| IFP534_PRG | QQHYHYH | SEQ ID NO: 6 | 4 | PRG |
| (SEQ ID NO: 64) | QQ | | | and |
| | | | | PEG |
| IFP538 | HQHYHYH | SEQ ID NO: 7 | 4 | GGGG |
| (SEQ ID NO: 66) | QH | | | |
| IFP540 | QQYHYHY | SEQ ID NO: 16 | 4 | GGGG |
| (SEQ ID NO: 68) | QQ | | | |
| IFP562 | QQEYHFH | SEQ ID NO: 26 | 5 | GGGG |
| (SEQ ID NO: 70) | YRQQ | | | |
| IFP563 | HQEYHFH | SEQ ID NO: 27 | 5 | GGGG |
| (SEQ ID NO: 72) | YRQH | | | |

Table 4 lists some representative IFPs that expressed well in E. coli. They were selected to show the range of variations of the core of His and Tyr-rich IFPs sequences. These variations included variations in the sequence of the SAPs, the length of the SAPs (here, 9, 11 and 13 amino acids), and the choice of linker separating the SAPs (here PRG and PEG or GGGG). Other IFPs, including SAP092 (SEQ ID NO: 15), SAP120 (SEQ ID NO: 19), SAP180 (SEQ ID NO: 18), SAP186 (SEQ ID NO: 13), SAP187 (SEQ ID NO: 24), SAP217 (SEQ ID NO: 17), SAP232 (SEQ ID NO: 5), SAP240 (SEQ ID NO: 16), SAP241 (SEQ ID NO: 8) and SAP242 (SEQ ID NO: 25) were also found to express well in E. coli.

To determine the soluble versus insoluble cell contents, cells from each of the 3 mL cultures were harvested by centrifugation and the pellet resuspended in 1 ml of 25 mM Tris 50 mM NaCl PH 7.5 buffer. The cell suspensions were transferred in 2 mL screw cap tubes containing 0.5 mL of ZrO2/SiO4 0.1 mm beads (Research Product International, Mount Prospect, IL) and disrupted in a FastPrep-24™ bead beater (MP Bio Irvine, CA) for 1 min at 5 m/s. After disruption, the cell extracts were transferred to new microfuge tubes. The soluble and insoluble fractions were then separated by centrifugation at 20,000×g for 5 min. The supernatants containing the soluble proteins were transferred to new tubes and the pellets containing the inclusion bodies were resuspended in the same volume of Tris-NaCl buffer.

Ten μL aliquots of the soluble fraction and of the particulate fraction are analyzed by SDS-PAGE using precast NuPAGE™ Bis-Tris 12% polyacrylamide gels (Thermo Scientific) as indicated by the manufacturer. After electrophoresis, the proteins and peptides were visualized by staining with GelCode™ Blue Safe Protein Coomassie Stain (Thermo Scientific).

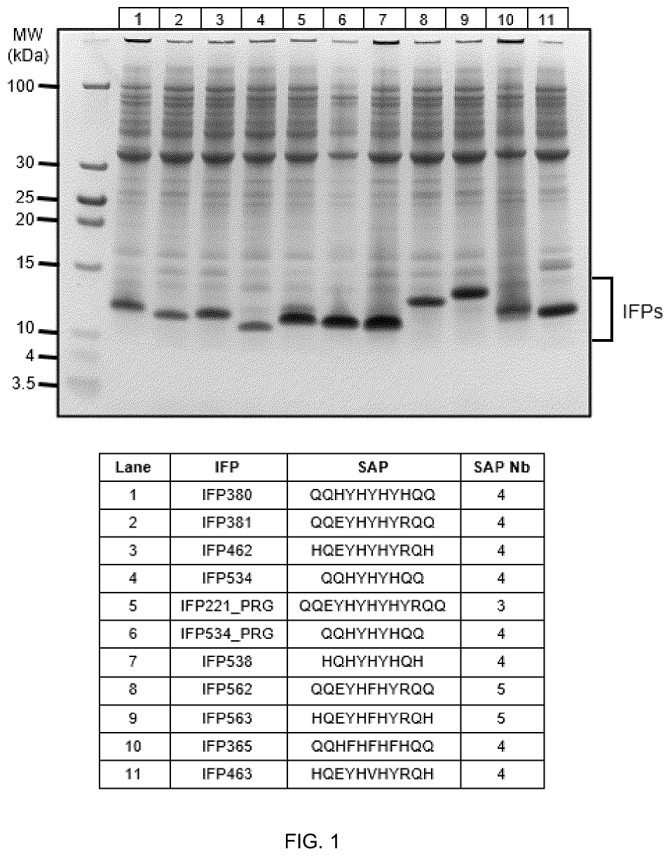

For some IFPs, no expression was observed in E. coli. However, for all IFPs that were expressed in E. coli, their expression was always insoluble. FIG. 1 shows a PAGE analysis of the insoluble fraction of E. coli extracts expressing a selection of IFPs. PAGE analysis provided a qualitative assessment of the insolubility of each IFP in the cell and enabled the identification of the most beneficial IFPs, fusions or IIPs.

The selection of IFP expressed insolubly shows that IFPs can include a variety of His and Tyr-rich SAPs, SAPs with a range of sizes (here 9, 11 or 13 amino acids), various of number of SAPs (here 3, 4 or 5) and with various linkers such as GGGG and PRG/PEG.

This example showed that IFPs with His and Tyr rich SAPs are expressed insolubly in E. coli.

### Example 3

### Assessment of the Solubility Properties of Insolubility Fusion Partners at Acidic and Alkaline pH

This example describes a general protocol used to assess the solubility of a polypeptide over a range of pH.

To evaluate the useful solubilization of the IFPs at acidic and alkaline pH (from pH 2 to pH 12), the insoluble fractions of cell extracts expressing selected IFPs were produced as described in Example 2. They were pelleted by centrifugation for 5 min at 20,000×g and washed with water several times to remove the buffer. The washed pellets were resuspended in 10 mM HCl pH 2 or in 10 mM NaOH pH 12. Suspensions were mixed vigorously and allowed to stand at room temperature for 15 min at which time they were centrifuged again for 5 min at 20,000×g. The solubilization of the IFPs in either acidic (pH 2) or alkaline (pH 12) conditions was assessed by PAGE analysis of an aliquot of the centrifugation supernatant.

All IFPs that were tested readily solubilized at both acidic and alkaline pHs demonstrating that the amino acid sequence of the SAPs determine the solubility of these IFPs under acidic and alkaline conditions, as well as their insolubility at pH close to neutrality.

Different IFPs that comprise different SAP sequences should have different solubility properties at different acidic pH or alkaline pH. This will enable the selection of the IFPs with the most useful solubility properties in order to construct fusion peptides to a POI for their insoluble microbial production and for the optimization of a purification process.

### Example 4

### Insoluble Expression of Simple Fusion Peptides

In this example, it is shown that a number of insolubility fusion partners (IFPs) based on His and Tyr-rich SAPs and expressed insolubly in the cell can be used for the production of peptides. The IFPs of interest are selected as described in Example 2.

Several IFPs were selected from the list in Table 4, and several prototypical POIs were tested. Some exemplary POIs include Lunasin, which is a 43-aa soy-derived peptide reported to have many health benefits [Fernández-Tome 2019]; Apidaecin, an 18-aa antimicrobial peptide [Torres 2019]; LL37, a 37-aa antimicrobial peptide derived from the human cathelicidin with numerous biological activities [Xhindoli 2019]; and Tachyplesin, a 17-aa antimicrobial peptide [U.S. Pat. No. 11,352,396].

As described above, numerous IFP-POI fusions were designed to include the following components assembled in this order:
an IFP with a core such as those described in Example 2;
a Gly rich linker such as GG, GGG or GGGG;
an acid cleavable linker such a DP, GGDP or DPGG;
a flexible linker such as GG, GGG or GGGG;
A POI or multiple copies of a POI;
a flexible linker such as GG, GGG or GGGG;
an acid cleavable linker such a DP, GGDP, DPGG, GGDPGG (SEQ ID NO: 37);
a flexible linker such as GG, GGG or GGGG; and
an optional C-terminal cap such as GPGP, PGPGP (SEQ ID NO: 40) or/and HHHHHH (SEQ ID NO: 39).

An exemplary, non-limiting list of suitable IFP-POI fusion peptides that expressed in an E. coli microbial host cell is shown in Table 5. For example, the sequence of IFP380-Api is:

(SEQ ID NO: 80)
MASGGGQQHYHYHYHQQGGGGQQHYHYHYHQ
QGGGGQQHYHYHYHQQGGGGQQHYHYHYHQQ
-GGGDPGGG-NNRPVYIPQPRPPHPRL-GGG
G-DP-GPGP

and that of IFP221-Apix2 is:

(SEQ ID NO: 94)
MASGGGQQEYHYHYHYRQQGGGGQQEYHYHY
HYRQQGGGGQQEYHYHYHYRQQ-GGGDPGGG-
NNRPVYIPQPRPPHPRL-GGGGDPGGG-NNR
PVYIPQPRPPHPRL-GGGG-DP-GPGP-HHH
HHH

TABLE 5
Representative IFP-POI fusions Expressed in E. coli

| Fusion | IFP | POI |
|---|---|---|
| IFP221_PRG-LL37 | IFP221_PRG | LL-37 |
| IFP221-Api | IFP221 | Apidaecin |
| (SEQ ID NO: 90) | | |
| IFP221-Apix2 | IFP221 | Apidaecin dimer |
| (SEQ ID NO: 94) | | |
| IFP221-LL37 | IFP221 | LL-37 |
| IFP380_PRG-LL37 | IFP380_PRG | LL-37 |
| IFP380-Api | IFP380 | Apidaecin |
| (SEQ ID NO: 80) | | |
| IFP380-Luna | IFP380 | Lunasin |
| (SEQ ID NO: 82) | | |
| IFP381-Api | IFP381 | Apidaecin |
| (SEQ ID NO: 88) | | |
| IFP381-Luna | IFP381 | Lunasin |
| (SEQ ID NO: 84) | | |
| IFP462-Api | IFP462 | Apidaecin |
| (SEQ ID NO: 74) | | |
| IFP462-Luna | IFP462 | Lunasin |
| (SEQ ID NO: 76) | | |
| IFP534-Api | IFP534 | Apidaecin |
| (SEQ ID NO: 86) | | |
| IFP534-LL37 | IFP534 | LL-37 |
| IFP534-Luna | IFP534 | Lunasin |
| (SEQ ID NO: 78) | | |
| IFP534-Tach | IFP534 | Tachyplesin |
| (SEQ ID NO: 92) | | |
| IFP538-LL37 | IFP538 | LL-37 |
| IFP634-LL37 | IFP634 | LL-37 |
| (SEQ ID NO: 96) | | |

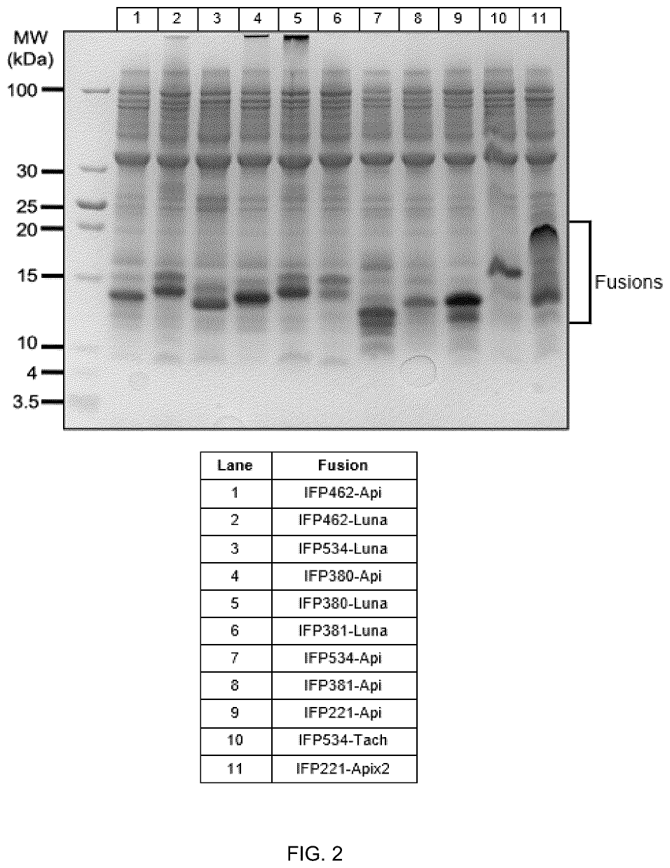

As described in Example 1, the genes encoding the IFP-POI fusion peptides were designed and cloned by the molecular biology service provider Twist Bioscience according to art standard synthesis techniques. The resulting expression plasmids were transferred in the E. coli expression strain BL21-AI and each strain was grown in the auto-induction medium for the production of peptides. The production of the fusion peptides in an insoluble form was assessed by the SDS-PAGE analysis of the insoluble fractions following cell lysis as described in Example 2. FIG. 2 shows the PAGE analysis of the insoluble fraction of cell extracts of E. coli expressing representative fusions listed above. All of the IFP-POI fusion peptides that were tested and that were express in the E. coli microbial host were soluble at pH 2 (see Example 6).

Thus, the fusion of peptides to IFPs that incorporate His and Tyr-rich SAPs express insolubly within the E. coli microbial host cell demonstrating the general utility of the novel IFPs that incorporate His and Tyr-rich SAPs.

### Example 5

### Insoluble Expression of Split Fusion Peptides

In addition to the fusion peptides discussed above that have a IFP-cLNK-POI core structure, alternative embodiments of fusion peptides in which the POI is “sandwiched” between IFPs were created and are sometimes referred to herein as “split fusions” or “split fusion peptides”. As demonstrated below, these split fusion peptides can be engineered for the insoluble production of a POI.

For example, IFP-Api-003 (SEQ ID NO: 98) is a split fusion peptide having the core structure IFP-cLNK-POI-cLNK-IFP with the POI (Apidaecin) sandwiched between two copies of IFP100 (SEQ ID NO: 119). Similarly, IFP-Api-006 (SEQ ID NO: 100) and IFP-Luna-006 (SEQ ID NO: 102) are split fusion peptides having the core structure IFP-cLNK-POI-cLNK-IFP-cLNK-POI-cLNK-IFP with two copies of the POI, either Apidaecin or Lunasin, sandwiched between three copies of IFP100 (i.e., IFP100-cLNK-POI-cLNK-IFP100-cLNK-POI-cLNK-IFP100). In these three split fusions, IFP100 is a small IFP consisting of only two copies of SAP234 (QQHYHYHQQ, SEQ ID NO: 6) separated by the GGGG spacer. The cleavable linker (cLNK) in these split fusion peptides is GGDPGG (SEQ ID NO: 37). Cells of E. coli containing a plasmid carrying the genes coding for IFP-Api-003 (SEQ ID NO: 99), IFP-Api-006 (SEQ ID NO: 101) or IFP-Luna-006 (SEQ ID NO: 103) all expressed the split fusion insolubly, demonstrating the flexibility of design for insoluble fusions based on His and Tyr-rich SAPs.

### Example 6

### Solubilization of Fusion Peptides and Removal of Host Contaminant Molecules

The following describes a method to purify fusion peptides, which were engineered to be soluble when the majority of host contaminating proteins are insoluble. All manipulations were carried out at a 1 mL scale without optimization of the process conditions for specific fusion peptides.

Representative fusion peptides were produced insolubly in 3 mL cultures as described in Example 1. Cells were harvested by centrifugation at 20,000×g, resuspended in 1 mL of 25 mM Tris 50 mM HCl pH 7.5 and disrupted with a bead beater as described in Example 2. Following cell disruption and centrifugation, the insoluble fractions were washed several times in deionized water to remove traces of buffer and then recovered by centrifugation. The fractions were resuspended in 1 mL of 10 mM HCl pH 2 with repeated vortexing over 15 min at room temperature. The soluble and insoluble acidified fractions were separated by centrifugation. at 20,000×g for 5 min The supernatants were transferred to new tubes and the pellets containing membranes and cell walls were resuspended in 1 mL of pH 7.5 Tris buffer. Aliquots of each fraction corresponding to a same initial volume of cell extracts were analyzed by SDS-PAGE.

It is expected that the fusion of peptides to IFPs that incorporated His and Tyr-rich SAPs and that expressed insolubly in the neutral environment of the cytoplasm would gain a positive charges at acidic pH and become soluble, while a large proportion of the E. coli proteins would precipitate due to their denaturation and the subsequent aggregation of their hydrophobic domains normally buried inside folded proteins. The fusion of peptides to IFP based on His-rich SAPs were found in the acid soluble fraction indicating that they resolubilized at acidic pH. The acid-insoluble fraction contained E. coli host contaminating proteins, in particular membranes as indicated by the glassy aspect of the pellet.

After 15 min at pH 2, the acidified supernatant fraction were neutralized by addition of 40 μL 500 mM Tris base or 60 μL of Bis-Tris base per mL of pH 2 fraction to reach a pH comprised between 6.5 and 8.0. For most fusions, the neutralization was accompanied by the immediate formation of a precipitate. When only a light precipitate was observed, the buffer was adjusted to neutrality by addition of HCl or of buffer base. The neutralized fractions were placed at −20° C. for 20 min and were subsequently thawed. The neutralized fractions were then centrifuged at 20,000×g for 5 min. Their supernatant was discarded and the pellets containing the insoluble fusion peptides resuspended in 1 ml of water.

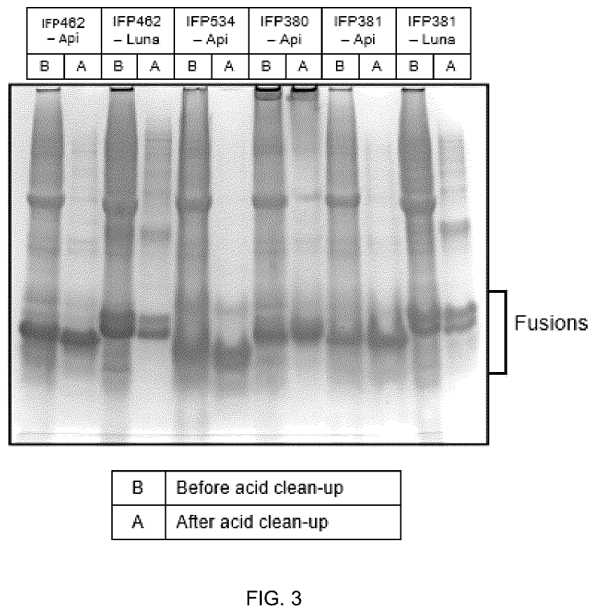

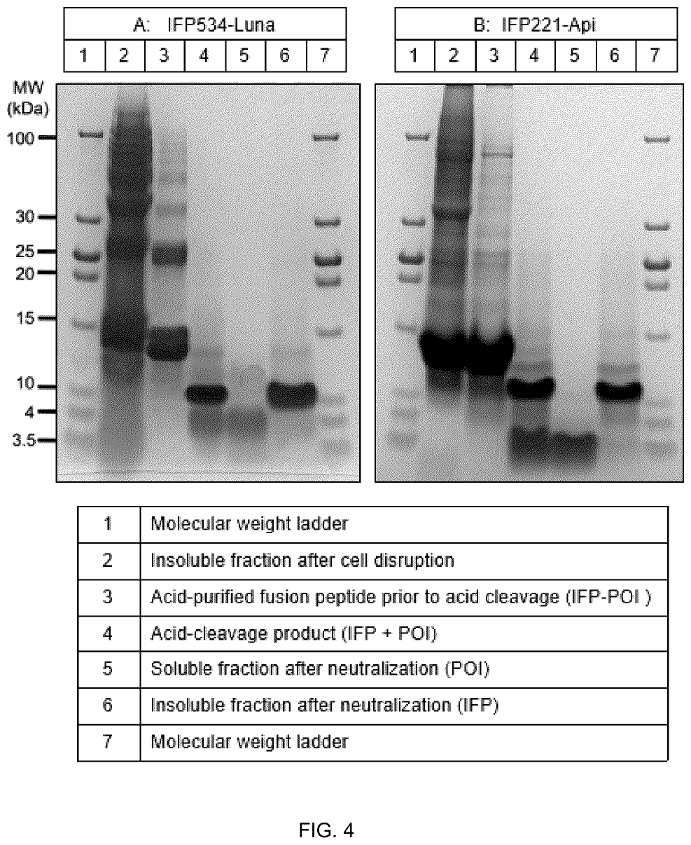

Examples of this acid-based clean-up/purification step of fusion peptides is shown in FIGS. 3 and 4 for exemplary fusion peptides IFP462-Api (SEQ ID NO: 74), IFP462-Luna (SEQ ID NO: 76), IFP534-Api (SEQ ID NO: 86), IFP380-Api (SEQ ID NO: 80), IFP381-Api (SEQ ID NO: 88), IFP381-Luna (SEQ ID NO: 84), IFP534-Luna (SEQ ID NO: 78), and IFP221-Api (SEQ ID NO: 90). In FIG. 3, the insoluble fractions after acid clean-up (lanes A) were significantly purer than the insoluble fraction obtained after cell disruption, but prior to acid clean-up (lanes B). Similarly, as shown in FIG. 4, the acid purified fractions for both the IFP534-Luna and IFP221-Api fusion peptides were significantly purer than the insoluble fraction obtained after cell disruption, but prior to the acid clean-up step (compare lanes 3 to lanes 2).

While not intending to be bound by theory, the inventor believes that since some of the fusion peptides having additional tyrosine residues will gain a negative charge at alkaline pH, these fusion peptides will become soluble while, at the same time, a portion of the E. coli basic proteins will precipitate due to their denaturation and the subsequent aggregation of their hydrophobic domains normally buried inside folded proteins. Under these conditions, the soluble fusion peptide could be separated from the precipitated host macromolecules by centrifugation.

In summary, the incorporation of multiple His and Tyr residues into the amino acid sequence of SAPs, especially for SAPs following Criterion 1, influences the solubility properties of the resulting IFPs containing these SAPs. In particular, these IFPs were shown: (1) to be insoluble at the neutral pH inside the microbial host cell, (2) to be soluble at acid pH and (3) to rapidly re-insolubilize when the pH is adjusted back to neutrality. Moreover, the solubility properties of the IFPs extended to IFP-POI fusion peptides, which can then be easily purified by the successive solubilization of the fusion proteins at acid pH, their physical separation from insoluble host proteins, and their recovery by rapid re-insolubilization following PH neutralization. Further, an alkaline purification step may be used in combination to purify the fusion peptide.

### Example 7

Cleavage and Separation of the Insolubility Fusion Partner from the Peptide of Interest

The following is a method for separating a peptide of interest from the insolubility fusion partner based on the solubility properties of the IFP.

After the acid-based purification step at acidic pH described in Example 5, the neutralized insoluble fusion peptides were washed again with water to remove traces of buffer and were resuspended in 1 mL of 10 mM HCl pH 2. The precipitates resolubilized immediately thus highlighting the rapid, reversible and pH-controllable properties of the peptide fusions to IFPs The tubes containing the solubilized fusion peptides were incubated at 80° C. for 4 hours to allow the cleavage of the Asp-Pro acid-labile peptide bond in the linker connecting the IFP and the POI.

After four hours at 80° C., the solutions were neutralized back to a pH comprised between 6.5 and 8.0 by addition of 60 μL of 500 mM Tris Base Tris per mL of pH 2 peptide solution. For most fusions, the neutralization was accompanied by the immediate formation of precipitates. When only a light precipitate was observed, the buffer was adjusted to neutrality by careful addition of HCl or of buffer base. The neutralized fractions were placed at −20° C. for 20 min to enhance precipitation, and then subsequently thawed and centrifuged at 20,000×g for 5 min. The supernatants were transferred to a new tube and the pellets resuspended in 1 ml of water. Aliquots of soluble and insoluble fractions were analyzed by PAGE.

FIG. 4 presents the purification process of two exemplary fusion peptides IFP534-Luna (SEQ ID NO: 78) and IFP221-Api (SEQ ID NO: 90). For both fusion peptides, the acid-purified fusion peptide before acid cleavage was observed at the expected molecular weight (see FIG. 4, lane 3; 12, 421 Da for IFP534-Luna and 10,680 for IFP221-Api). The ladder observed for IFP534-Luna may represent aggregates that collapse upon incubation at 80° C. at pH 2. After incubation for 4 hours at 80° C., the acid-cleaved IFP and POI were separated on the gel as two distinct bands (lanes 4) migrating around the expected MW (6,451 Da for IFP534, 5,583 Da for Luna, 6,807 Da for IFP221, and 2,663 Da for Api).

Following neutralization and centrifugation, the soluble POIs partitioned in the supernatant (lanes 5) whereas the insoluble IFPs partitioned in the pellets (lanes 6). Notably, the Luna peptide carrying a 9 Asp tail did not stain well with the Coomassie dye presumably due to the dye's own negatively charge. At the end of this process, the POIs were recovered in a highly purified form (i.e., separated from both the host cell proteins and from the IFP) without requiring a chromatographic step, high concentration of chemicals, or expensive cleavage enzymes. As such, this purification process is highly scalable.

Thus, as shown above, IFPs comprising His and Tyr-rich pH-responsive SAPs can be selected for compatibility with a specific POI to produce the purified POI. Further, this process exploits the respective solubility properties of the IFP-POI fusion to provide a useful method for the bioproduction of soluble peptides at high purity, without the need for costly chromatographic steps.

### Example 8

### Insoluble Bioproduction of Immunogenic Peptides Displayed in Self-Aggregating Structures

This example describes the production of various architectures of Insoluble Immunogenic Peptides (IIPs) displayed in self-aggregating structures.

IIPs that include combinations of immunogenic epitopes and SAPs with desirable solubility properties were designed to have structures such as those represented in FIG. 5, FIG. 6 and FIG. 8. Such IIPs that incorporate different SAPs and different architectures are described in Table 6. The gene design, DNA synthesis, molecular cloning into the T7 expression vector pET-29 (+) were carried out by the molecular biology service company Twist Bioscience as in Example 1. Strains of BL21-AI carrying a pET-29 (+) expression plasmid encoding for IIP genes were constructed, grown, harvested and disrupted as described in Example 1. Representative IIPs are shown in Table 6.

TABLE 6
Representative Insoluble Immunogenic Peptides Produced in E. coli

| IIP | Reference to | | SAP Copy Number | Copy Number |
|---|---|---|---|---|
| (SEQ ID NO) | Figures | SAP | with SAP Sequence | with POI |
| Cleaved | FIG. 5 | SAP162 | 2 × HQEYHYHYRQH | 1 × Ova1 |
| IIP003 | Structure 3 | | | |
| Cleaved | FIG. 5 | SAP234 | 2 × QQHYHYHQQ | 1 × Ova1 |
| IIP015 | Structure 3 | | | |
| IIP004 | FIG. 5 | SAP162 | 3 × HQEYHYHYRQH | 1 × Ova1 |
| (SEQ ID NO: 108) | Structure 4 | | | |
| IIP011 | FIG. 5 | SAP081 | 4 × QQEYHYHYRQQ | 1 × Ova1 |
| (SEQ ID NO: 110) | Structure 5 | | | |
| IIP014 | FIG. 5 | SAP234 | 4 × QQHYHYHQQ | 1 × Ova1 |
| (SEQ ID NO: 112) | Structure 5 | | | |
| IIP003 | FIG. 8 | SAP162 | 6 × HQEYHYHYRQH | 3 × Ova1 |
| (SEQ ID NO: 104) | Structure 9 | | | |
| IIP015 | FIG. 8 | SAP234 | 6 × QQHYHYHQQ | 3 × Ova1 |
| (SEQ ID NO: 114) | Structure 9 | | | |
| IIP006 | FIG. 6 | SAP162 | 6 × HQEYHYHYRQH | 5 × Ova1 |
| (SEQ ID NO: 106) | Structure 7 | | | |
| IIP019 | FIG. 6 | SAP081 | 6 × QQEYHYHYRQQ | 1 × each of Ova1, |
| (SEQ ID NO: 116) | Structure 8 | | | RS09, TPD, |
| | | | | PADRE, Ova2 |

Some of the exemplary peptides listed in Table 6 include the immunogenic epitope for OVA1 (SEQ ID NO: 45); the immunogenic epitope for OVA2 (SEQ ID NO: 46); the immunogenic epitope for RS09 (SEQ ID NO: 47); the immunogenic epitope for TPD (SEQ ID NO: 48); and the immunogenic epitope for PADRE (SEQ ID NO: 49). Each of the POIs are linked to one or more copies of an SAP sequence as exemplified in Table 6. These IIPs start with the MAS translation initiation codon that is separated from the first SAP by the acid cleavable linker GGDPGG (SEQ ID NO: 37). The IIPs are capped at their C-terminus by the Pro-rich sequence PGPGP (SEQ ID NO: 40), which is separated from the last SAP by the acid cleavable linker GGDPGG. For example, IIP004 comprises a single Ova1 POI and two copies of the SAP162 (HQEYHYHYRQH; SEQ ID NO: 12) represented by Structure 3 (FIG. 5).

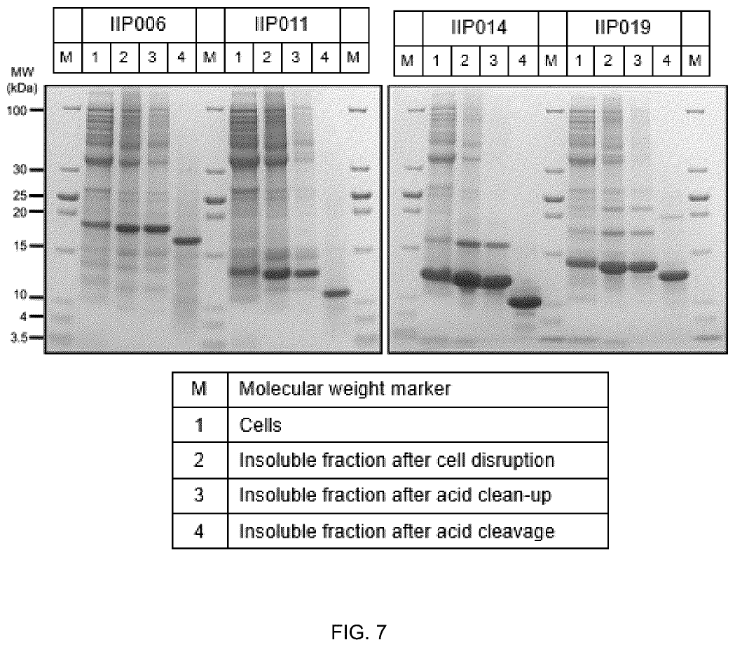

The solubility of polypeptides was characterized as described in Example 3. Presumably because of the His-Tyr-rich SAPs included in these peptides, each of the IIPs listed in Table 6 expressed insolubly in E. coli. The insoluble fraction of the cell extracts were washed in water several times and resuspended in 10 mM HCl to pH 2. The IIPs resolubilized readily and were separated from insoluble denatured host proteins and membranes by centrifugations as described in Example 5. The soluble fractions in the acidified supernatants were then brought back to neutrality by addition of Tris-Base causing the rapid precipitation of the IIPs, which were recovered in the pellet after centrifugation. The purification of four IIPs representing several IIP structures (IIP006, 011, 014 and 019) are depicted in FIG. 7 (lanes 3 versus lanes 2). It is hypothesized by the inventor that additional cycles of re-solubilization and insolubilization by various pH shifts and appropriate addition of salts will lead to even greater purity of the IIPs.

Further purification could be achieved by the removal of the N-terminal MAS translation initiation peptide as well as the C-terminal cap PGPGP using acid cleavage at the heat labile Asp-Pro sequences as described in Example 6. After 4 hours at 80° C. and pH 2, the pH of the solution was brought back to neutrality by addition of 500 mM Tris base and the IIPs were recovered in the insoluble fraction. FIG. 7 shows the increase in purity of the IIPs and their decrease in molecular weight as expected following the removal of the MAS and of the PGPGP C-terminal cap (lanes 4 versus lanes 3).

In summary, the results discussed herein demonstrated: (1) the biological expression of insoluble immunogenic peptides inside a microbial host; (2) the purification of these peptides from host proteins as a scalable method that does not require a chromatographic step, high concentration of chemicals, or expensive cleavage enzymes; and (3) the inclusion of immunogenic epitopes within self-aggregating structures for the production of biomaterials and vaccines. These three functionalities derive from the incorporation of His and Tyr-rich SAPs in the structure of the IIPs Further, the use of this approach for various IIP architectures with a single epitope or each copy of the epitope connected to one or more SAPs (e.g., as shown in FIG. 5 and FIG. 6) can be produced efficiently and form supra-molecular aggregates.

### Example 9

### Design and Bioproduction of Immunogenic Peptides Displaying Multiple Epitopes

In some applications, such as for the production of vaccines, it may be beneficial to display multiple immunogenic, targeting or recognition peptide sequences in one single long polypeptide. This approach may increase the avidity of an epitope toward its receptor by displaying multiple copies of a single epitope (e.g., IIP109 and IIP110) or it may increase the strength of an immune response by displaying different epitopes stimulating different types of cells that contribute to the overall immune response (e.g., IIP019). To this end, described herein is the design and the production of exemplary multi-epitope peptides based on the properties of the self-aggregating peptides (SAPs) they comprise.

To illustrate, the exemplary multi-epitope IIP019 was designed to comprise the prototypical self-aggregating peptide SAP081 (QQEYHYHYRQQ) To demonstrate the production of IIPs that include multiple and distinct epitopes, epitope RS09 (Tol-like receptor TLR4 agonist), epitope TPD (a chimeric T-helper MHC class II epitope), and PADRE (a T-helper epitope) were used in addition to the immunogenic OVA1 and OVA2 epitopes. The structure of the IIP019 is represented by Structure 8 (FIG. 6). The purification of IIP019 is shown in FIG. 7 (lane 4).

As can be seen by the working examples discussed herein, the insoluble multi-epitope immunological peptides were able to be produced insolubly inside the microbial host cells. Further, the peptides were amenable to acid-based clean-up to yield a high purity peptide via a purification process based only on physical separation methods and ultimately relying on the solubility properties of the His and Tyr-rich SAPs included in these peptides. The display of distinct immunogenic epitopes displayed in one self-aggregating structure can enable applications in vaccines and bio-materials.

### Example 10

Production of Immunogenic Peptides Displayed in Self-Aggregating Structures from Catenated Precursors

Expression and purification was carried out for catenated copies of an immunogenic peptide displayed in self-aggregating structures, which were then subsequently separated into individual, simpler IIP units comprising a single epitope peptide sandwiched between one or more self-aggregating peptides (SAPs).

IIPs that incorporated multiple catenated copies of the immunogenic epitope Ova1 with the architecture represented as structure 9 (FIG. 8) were designed with His and Tyr-rich SAPs. For instance, IIP003 (SEQ ID NO: 104) and IIP015 (SEQ ID NO: 114) each incorporated three copies of Ova1 interspersed between six copies of SAP162 (HQEYHYHYRQH, SEQ ID NO: 12) and SAP234 (QQHYHYHQQ, SEQ ID NO: 6), respectively. For both IIPs, an Asp-Pro cleavable linker separates the simple structures SAP-POI-SAP (structure 3).

Optimized genes coding for the amino acid sequence of IIP003 and IIP015 were designed, synthesized and cloned in the expression plasmid pET-29 (+) by Twist Bioscience. The plasmids were transferred into E. coli BL21-AI and grown in auto-induction medium as described in Example 1. The longer catenated peptides were produced insolubly inside the cell and were purified as described in Examples 2 to 5. After removal of the acid-precipitated contaminating host macromolecules from the solubilized peptide as described in Example 5, the IIP-containing fraction was again acidified to pH 2 by addition of 10 mM HCl and incubated at 80° C. as described in Example 6. After four hours, the solution was brought back to neutral pH by addition of 0.5 M Tris Base followed by the rapid precipitation of peptides. The precipitated fraction was recovered by centrifugation and resuspended in Tris-NaCl buffer. The visualization of the peptide clean-up and cleavage was monitored at all steps of the process by SDS-PAGE.

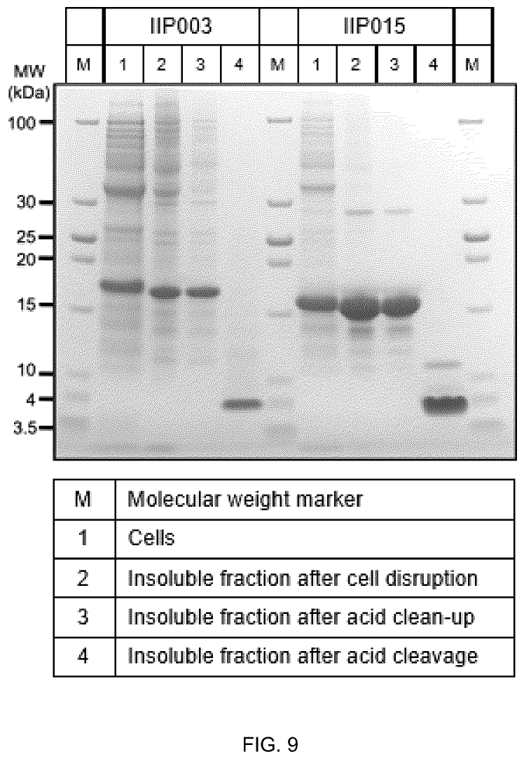

FIG. 9. shows the purification of the catenated peptides of IIP003 and IIP015 exploiting the solubilization of these peptides at acidic pH, their separation from acid-insoluble denatured host proteins and membranes, and their re-insolubilization upon neutralization resulting in a significant increase in peptide purity (lanes 3 vs lanes 1 and 2). FIG. 9 also shows the release of the homogeneous shorter peptides with structure 3 (FIG. 5) following acid cleavage at 80° C. for 4 hours. This step also removed the N-terminal translation initiation peptide as well as the C-terminal cap. The simpler structure 3 peptides (SAP—POI-SAP) with a lower molecular weight were also insoluble (lanes 4 s lanes 3) presumably due to the solubility properties of their respective SAPs.

As demonstrated herein, short IIPs can be produced from larger catenated IIPs at high purity with a purification process that uses only physical separation methods based on the controllable solubility properties of the His and Tyr-rich SAPs incorporated within the fusion peptide.

### REFERENCES

### Us Patent Documents

| Patent No | Priority date | First inventor |
|---|---|---|
| 5,648,244 | September 1993 | Kuliopoulos et al. |
| 6,037,145 | July 1997 | Yabuta et al. |
| 7,416,862 | August 1997 | Doucette-Stamm et |
| 7,595,173 | April 2002 | Krebs et al. |
| 7,662,913 | October 2006 | Decarolis et al. |
| 7,678,883 | July 2007 | Cheng et al. |
| 7,732,569 | June 2004 | Decarolis et al. |
| 7,749,731 | March 1999 | Better et al. |
| 7,794,979 | July 2007 | Cheng et al. |
| 7,951,559 | July 2007 | Alsop et al. |
| 8,575,110 | February 2003 | Burkhard |
| 8,796,431 | November 2009 | Stowell et al. |
| 9,062,312 | June 2011 | Rouviere et al. |
| 9,162,005 | May 2005 | Ellis-Behnke et al. |
| 9,200,306 | October 2012 | Lin et al. |
| 9,951,368 | May 2002 | Williams et al. |
| 10,245,318 | September 2014 | Burkhard et al. |
| 10,443,081 | April 2013 | Schmitt et al. |
| 10,596,238 | November 2009 | Collier et al. |
| 11,352396 | December 2017 | Kuhn et al. |

### Non-Patent Literature

Aggeli A., Bell, M., Boden, N., Keen, J., & McLeish, T. B. (1997). Engineering of peptide β-sheet nanotapes. Journal of Materials Chemistry, 7 (7), 1135-1145.
Aggeli, A., Nyrkova, I. A., Bell, M., Harding, R., Carrick, L., Mcleish, T. C., . . . & Boden, N. (2001). Hierarchical self-assembly of chiral rod-like molecules as a model for peptide β-sheet tapes, ribbons, fibrils, and fibers. Proceedings of the National Academy of Sciences, 98 (21), 11857-11862.
Aggeli, A., Bell, M., Carrick, L. M., Fishwick, C. W., Harding, R., Mawer, P. J., . . . & Boden, N. (2003). pH as a trigger of peptide β-sheet self-assembly and reversible switching between nematic and isotropic phases. Journal of the American Chemical Society, 125 (32), 9619-9628.
Alexander, J., Sidney, J., Southwood, S., Ruppert, J., Oseroff, C., Maewal, A., . . . & Grey, H. M. (1994). Development of high potency universal DR-restricted helper epitopes by modification of high affinity DR-blocking peptides. Immunity, 1 (9), 751-761.
Bell, M. R., Engleka, M. J., Malik, A., & Strickler, J. E. (2013). To fuse or not to fuse: What is your purpose?. Protein Science, 22 (11), 1466-1477.
Chen, P. (2005). Self-assembly of ionic-complementary peptides: a physicochemical viewpoint. Colloids and Surfaces A: Physicochemical and Engineering Aspects, 261 (1-3), 3-24.
Cheng, J., & Baldi, P. (2005). Three-stage prediction of protein β-sheets by neural networks, alignments and graph algorithms. Bioinformatics, 21 (suppl. 1), i75-i84.
DePalma, A. (2015). Peptides: New processes, lower costs. Genetic Engineering & Biotechnology News, 35 (13), 24-26.
Diaz-Caballero, M., Navarro, S., Nuez-Martinez, M., Peccati, F., Rodriguez-Santiago, L., Sodupe, M., . . . & Ventura, S. (2020). pH-Responsive Self-Assembly of Amyloid Fibrils for Dual Hydrolase-Oxidase Reactions. ACS Catalysis, 11 (2), 595-607.
Ding, Y., Liu, J., Lu, S., Igweze, J., Xu, W., Kuang, D., . . . & Zhang, L. (2016). Self-assembling peptide for co-delivery of HIV-1 CD8+ T cells epitope and Toll-like receptor 7/8 agonists R848 to induce maturation of monocyte derived dendritic cell and augment polyfunctional cytotoxic T lymphocyte (CTL) response. Journal of Controlled Release, 236, 22-30.
El Bissati, K., Zhou, Y., Dasgupta, D., Cobb, D., Dubey, J. P., Burkhard, P., . . . & McLeod, R. (2014). Effectiveness of a novel immunogenic nanoparticle platform for Toxoplasma peptide vaccine in HLA transgenic mice. Vaccine, 32 (26), 3243-3248.
Fernández-Tome, S., & Hernandez-Ledesma, B. (2019). Current state of art after twenty years of the discovery of bioactive peptide lunasin. Food research international, 116, 71-78.
Família, C., Dennison, S. R., Quintas, A., & Phoenix, D. A. (2015). Prediction of peptide and protein propensity for amyloid formation. PloS one, 10 (8), e0134679.
Fraser, C. C., Altreuter, D. H., Ilyinskii, P., Pittet, L., LaMothe, R. A., Keegan, M., . . . & Kishimoto, T. K. (2014). Generation of a universal CD4 memory T cell recall peptide effective in humans, mice and non-human primates. Vaccine, 32 (24), 2896-2903.
Gaglione, R., Pane, K., Dell′Olmo, E., Cafaro, V., Pizzo, E., Olivieri, G., . . . & Arciello, A. (2019). Cost-effective production of recombinant peptides in Escherichia coli. New biotechnology, 51, 39-48.
Garulli, B., Di Mario, G., Sciaraffia, E., Kawaoka, Y., & Castrucci, M. R. (2011). Immunogenicity of a Recombinant Influenza Virus Bearing Both the CD4. BioMed Research International, 2011.
Gibson, D. G., Young, L., Chuang, R. Y., Venter, J. C., Hutchison, C. A., & Smith, H. O. (2009). Enzymatic assembly of DNA molecules up to several hundred kilobases. Nature methods, 6 (5), 343-345.
Hayakawa, T., Sato, S., Iwamoto, S., Sudo, S., Sakamoto, Y., Yamashita, T., . . . & Sakai, H. (2010). Novel strategy for protein production using a peptide tag derived from Bacillus thuringiensis Cry4Aa. The FEBS journal, 277 (13), 2883-2891.
Ingham, A. B., & Moore, R. J. (2007). Recombinant production of antimicrobial peptides in heterologous microbial systems. Biotechnology and applied biochemistry, 47 (1), 1-9.
Isidro-Llobet, A., Kenworthy, M. N., Mukherjee, S., Kopach, M. E., Wegner, K., Gallou, F., . . . & Roschangar, F. (2019). Sustainability challenges in peptide synthesis and purification: From R&D to production. The Journal of organic chemistry, 84 (8), 4615-4628.
Ito, M., Hayashi, K., Minamisawa, T., Homma, S., Koido, S., & Shiba, K. (2017). Encryption of agonistic motifs for TLR4 into artificial antigens augmented the maturation of antigen-presenting cells. PloS one, 12 (11), e0188934.
Kaba, S. A., McCoy, M. E., Doll, T. A., Brando, C., Guo, Q., Dasgupta, D., . . . & Burkhard, P. (2012). Protective antibody and CD8+ T-cell responses to the Plasmodium falciparum circumsporozoite protein induced by a nanoparticle vaccine. PLOS One, 7 (10), e48304.
Karch, C. P., & Burkhard, P. (2016). Vaccine technologies: from whole organisms to rationally designed protein assemblies. Biochemical pharmacology, 120, 1-14.
Kota, S. (2019). Peptide Manufacturing Methods and Challenges. Peptide Therapeutics: Strategy and Tactics for Chemistry, Manufacturing, and Controls, 72, 111.
Lamiable, A., Thévenet, P., Rey, J., Vavrusa, M., Derreumaux, P., & Tufféry, P. (2016). PEP-FOLD3: faster de novo structure prediction for linear peptides in solution and in complex. Nucleic acids research, 44 (W1), W449-W454.
LaVallie, E. R., McCoy, J. M., Smith, D. B., & Riggs, P. (1994). Enzymatic and chemical cleavage of fusion proteins. Current Protocols in Molecular Biology, 28 (1), 16-4.
Lax, E. R., & Shah, T. (2019). Economic and Environmental Factors Affecting the Sustainability of Peptide Therapeutic Manufacturing. Peptide Therapeutics: Strategy and Tactics for Chemistry, Manufacturing, and Controls, 72, 151.
Lei, H., Zhang, X., Hu, J., & Zhang, Y. (2017). Self-assembly of amyloid-like peptides at interfaces investigated by atomic force microscopy. Science of Advanced Materials, 9 (1), 65-76.
Negahdaripour, M., Nezafat, N., Heidari, R., Erfani, N., Hajighahramani, N., Ghoshoon, M. B., . . . & Morowvat, M. H. (2020). Production and Preliminary in vivo Evaluations of a Novel in silico-Designed L2-based Potential HPV Vaccine. Current Pharmaceutical Biotechnology, 21 (4), 316-324.
Parachin, N. S., Mulder, K. C., Viana, A. A. B., Dias, S. C., & Franco, O. L. (2012). Expression systems for heterologous production of antimicrobial peptides. Peptides, 38 (2), 446-456.
Seale, J. W. (2006). The role of a conserved histidine-tyrosine interhelical interaction in the ion channel domain of δ-endotoxins from Bacillus thuringiensis. Proteins: Structure, Function, and Bioinformatics, 63 (2), 385-390.
Shanmugam, A., Rajoria, S., George, A. L., Mittelman, A., Suriano, R., & Tiwari, R. K. (2012). Synthetic Toll like receptor-4 (TLR-4) agonist peptides as a novel class of adjuvants. PloS one, 7 (2).
Smith, C. K., & Regan, L. (1997). Construction and design of β-sheets. Accounts of chemical research, 30(4), 153-161.
Studier, F. W. (2005). Protein production by auto-induction in high-density shaking cultures. Protein expression and purification, 41 (1), 207-234.
TerMaat, J. R., Pienaar, E., Whitney, S. E., Mamedov, T. G., & Subramanian, A. (2009). Gene synthesis by integrated polymerase chain assembly and PCR amplification using a high-speed thermocycler. Journal of microbiological methods, 79 (3), 295-300.
Thermo-Fisher (2015). Technical literature: GeneArt Gene Synthesis and services.
Thermo-Fisher (2020). Technical literature: GeaneArt Gene Synthesis Handbook.
Thermo-Fisher (2015). Technical literature: GeneArt Gene to Protein Handbook.
Thermo Scientific (2015). Protein expression handbook: Recombinant protein expression and purification technologies.
Torres, M. D. T., & de la Fuente-Nunez, C. (2019). Reprogramming biological peptides to combat infectious diseases. Chemical Communications, 55 (100), 15020-15032.
Tsoras, A. N., & Champion, J. A. (2019). Protein and peptide biomaterials for engineered subunit vaccines and immunotherapeutic applications. Annual review of chemical and biomolecular engineering, 10, 337-359.
Walsh, I., Seno, F., Tosatto, S. C., & Trovato, A. (2014). PASTA 2.0: an improved server for protein aggregation prediction. Nucleic acids research, 42 (W1), W301-W307.
Wibowo, D., & Zhao, C. X. (2019). Recent achievements and perspectives for large-scale recombinant production of antimicrobial peptides. Applied microbiology and biotechnology, 103 (2), 659-671.
Woolfson, D. N., & Mahmoud, Z. N. (2010). More than just bare scaffolds: towards multi-component and decorated fibrous biomaterials. Chemical Society Reviews, 39 (9), 3464-3479.
Wu, W., Xing, L., Zhou, B., & Lin, Z. (2011). Active protein aggregates induced by terminally attached self-assembling peptide ELK16 in Escherichia coli. Microbial cell factories, 10 (1), 9.
Xhindoli, D., Pacor, S., Benincasa, M., Scocchi, M., Gennaro, R., & Tossi, A. (2016). The human cathelicidin LL37-A pore-forming antibacterial peptide and host-cell modulator. Biochimica et Biophysica Acta (BBA)-Biomembranes, 1858 (3), 546-566.
Yang, X., Pistolozzi, M., & Lin, Z. (2018). New trends in aggregating tags for therapeutic protein purification. Biotechnology letters, 40 (5), 745-753.
Zhang, S., Guo, F., Yan, W., Dai, Z., Dong, W., Zhou, J., . . . & Jiang, M. (2020). Recent advances of CRISPR/Cas9-based genetic engineering and transcriptional regulation in industrial biology. Frontiers in Bioengineering and Biotechnology, 7, 459.
Zhao, Q., Xu, W., Xing, L., & Lin, Z. (2016). Recombinant production of medium-to large-sized peptides in Escherichia coli using a cleavable self-aggregating tag. Microbial cell factories, 15(1), 136.
Zorko, M., & Jerala, R. (2010). Production of recombinant antimicrobial peptides in bacteria. In Antimicrobial Peptides (pp. 61-76). Humana Press, Totowa, NJ.

The invention claimed is:

1. A peptide comprising 2n or 2n+1 amino acids wherein at least n−1 amino acids are selected from the group consisting of His and Tyr, and wherein said peptide self-aggregates at neutral pH between pH 6 and pH 8.

2. (canceled)

3. The peptide of claim 1, comprising an amino acid sequence of alternating histidines and tyrosines according to the formula (Tyr)p-(His-Tyr)n-(His)q (Formula 1), wherein:
p is a number equal to 0 or 1;
q is a number equal to 0 or 1;
n is a number equal to or greater than 3;
and wherein said peptide self-aggregates at neutral pH between pH 6 and pH 8.

4. (canceled)

5. (canceled)

6. The peptide of claim 1, wherein:
(i) the peptide is not charged at neutral pH; or
(ii) n is a number equal to or greater than 5; or
(iii) the peptide is biologically produced; or
(iv) any combination of (i)-(iii).

7. The peptide of claim 1, comprising an amino acid sequence selected from the group consisting of SEQ ID NO: 2, SEQ ID NO: 3, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35.

8. The peptide of claim 1, comprising an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35, wherein the peptide is biologically produced.

9. The peptide of claim 1, comprising an amino acid sequence selected from the group consisting of SEQ ID NO: 2, SEQ ID NO: 3, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35.

10. An insolubility fusion partner comprising the structure [SAP]-[[Spacer]-[SAP]]m wherein;
a) the SAP is a peptide comprising 2n or 2n+1 amino acids wherein at least n−1 amino acids are selected from the group consisting of His and Tyr, and wherein said peptide self-aggregates at neutral pH between pH 6 and pH 8;
b) the spacer is a peptide having from 1 to 50 amino acids;
c) m is an integer from 0 to 10;
and wherein said insolubility fusion partner is insoluble at neutral pH between pH 6 and pH 8.

11. The insolubility fusion partner of claim 10, comprising 60 amino acids or less.

12. The insolubility fusion partner of claim 10, wherein the fusion partner is soluble:
(i) at a pH of less than 5; or
(ii) at a pH of greater than 10; or
(iii) at a pH of less than 5 and at a pH of greater than 10.

13. (canceled)

14. The insolubility fusion partner of claim 10, comprising an amino acid sequence selected from the group consisting of SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, and SEQ ID NO:119.

15. A method for producing and recovering a peptide of interest comprising:
a) providing a genetically engineered microbial host cell that expresses a genetic construct encoding an amino acid sequence of a fusion peptide, wherein said fusion peptide comprises:
i) an insolubility fusion partner that is insoluble at neutral pH and soluble at acid pH;
ii) a linker comprising one or more cleavage sites; and
iii) a peptide of interest that is soluble at neutral pH;
b) growing the microbial host under conditions wherein said fusion peptide is produced in an insoluble form in the host cytoplasm;
c) recovering the insoluble fusion peptide of step b);
d) solubilizing the recovered insoluble fusion peptide of step c) in an acidic medium;
e) recovering the solubilized fusion peptide of step d);
f) cleaving the insolubility fusion partner from the peptide of interest; and
g) altering the pH to precipitate the insoluble fusion partner and recovering the peptide of interest in soluble form.

16. (canceled)

17. (canceled)

18. The method of claim 15, wherein the insoluble fusion peptide is recovered at step c), step e), or both step c) and step e) by centrifugation, filtration, sedimentation, or combination thereof.

19. (canceled)

20. The method of claim 15, wherein
i) the fusion peptide of step a) comprises an insolubility fusion partner which is soluble at a pH of less than 5 or greater than 10, but insoluble at neutral pH between pH 6 and pH 8;
ii) after step d) the solubilized fusion peptide is separated from insoluble fractions by physical separation and resolubilized in a medium at a pH of greater than 10; and
iii) the resolubilized fusion peptide of step ii) is further separated from insoluble fractions by physical separation.

21. (canceled)

22. A fusion peptide with a structure according to the formula:
[[SAP-CON-]m-POI-[CON-SAP]n-CON]]p
wherein:
(a) p is an integer greater than 0;
(b) m is an integer including 0, wherein in any repeating segment represented by p, m can be the same integer or m can be a different integer from any other repeating segment represented by p;
(c) n is an integer including 0, wherein in any repeating segment represented by p, n can be the same integer or n can be a different integer from any other repeating segment represented by p;
(d) SAP is a self-aggregating peptide comprising 2n or 2n+1 amino acids wherein at least n−1 amino acids are selected from the group consisting of His and Tyr, and wherein said peptide self-aggregates at neutral pH between pH 6 and pH 8;
(e) POI is a peptide of interest comprising more than one epitope amino acid sequence; and
(f) CON is (i) a connecting peptide and may represent more than one connector amino acid sequence; or (ii) is absent from the structure.

23. (canceled)

24. (canceled)

25. The fusion peptide of claim 22, wherein:
m is 1, n is 0, and p is 1; or
m is 0, n is 1, and p is 1; or
m is 2, n is 0, and p is 1; or
m is 0, n is 2, and p is 1; or
m is 1, n is 1, and p is 1; or
m is 2, n is 1, and p is 1; or
m is 2, n is 2, and p is 1; or
m is 1, n is 1, and p is 3; or
m is 1, n is 0, and p is 5.

26. The fusion peptide of claim 22, wherein the SAP is a self-aggregating peptide comprising an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35.

27. The fusion peptide of claim 22, wherein the SAP is insoluble at a pH between about 6 to about 8.

28. The fusion peptide of claim 22, wherein the fusion peptide is bioactive or immunogenic.

29. A method for producing an insoluble immunogenic or bioactive peptide, comprising genetically engineering a microbial host genetically to comprise a nucleic acid segment coding for an immunogenic or bioactive peptide that is insoluble at neutral pH and soluble under acidic conditions, wherein said immunogenic or bioactive peptide comprises the fusion peptide of claim 22.

30. The method of claim 29, further comprising
a) growing the genetically engineered microbial host cell under conditions where the genetic construct is expressed and the encoded immunogenic or bioactive peptide is produced in an insoluble form in the cytoplasm of the host cell;
b) recovering the insoluble peptide after physical separation from soluble host cell components;
c) subjecting the insoluble immunogenic or bioactive peptide to an aqueous medium having a pH at which said insoluble immunogenic or bioactive peptide becomes soluble;
d) recovering the solubilized immunogenic or bioactive peptide in the aqueous phase after physical separation of insoluble host cell components;
e) adjusting the composition of the solution containing the immunogenic or bioactive peptide to a neutral pH at which it becomes insoluble; and
f) recovering the insoluble immunogenic or bioactive peptide after physical separation.


SEQUENCE LISTING (119 sequences)

SEQ ID NO: 1 - protein, 4 aa, synthetic construct
  REPEAT 1: note = p is a number equal to 0 or 1
  REPEAT 2..3: note = n is a number equal to or greater than 3
  REPEAT 4: note = q is a number equal to 0 or 1
YHYH

SEQ ID NO: 2 - protein, 11 aa, synthetic construct
HYHYHYHYHYH

SEQ ID NO: 3 - protein, 13 aa, synthetic construct
HYHYHYHYHYHYH

SEQ ID NO: 4 - protein, 9 aa, synthetic construct
HYHYHYHYH

SEQ ID NO: 5 - protein, 7 aa, synthetic construct
HYHYHYH

SEQ ID NO: 6 - protein, 9 aa, synthetic construct
QQHYHYHQQ

SEQ ID NO: 7 - protein, 9 aa, synthetic construct
HQHYHYHQH

SEQ ID NO: 8 - protein, 9 aa, synthetic construct
QHYHYHYHQ

SEQ ID NO: 9 - protein, 11 aa, synthetic construct
QQHYHYHYHQQ

SEQ ID NO: 10 - protein, 11 aa, synthetic construct
QQEYHYHYRQQ

SEQ ID NO: 11 - protein, 13 aa, synthetic construct
QQEYHYHYHYRQQ

SEQ ID NO: 12 - protein, 11 aa, synthetic construct
HQEYHYHYRQH

SEQ ID NO: 13 - protein, 11 aa, synthetic construct
QQHYHAHYHQQ

SEQ ID NO: 14 - protein, 11 aa, synthetic construct
QQRYHYHYEQQ

SEQ ID NO: 15 - protein, 11 aa, synthetic construct
HQRYHYHYEQH

SEQ ID NO: 16 - protein, 9 aa, synthetic construct
QQYHYHYQQ

SEQ ID NO: 17 - protein, 13 aa, synthetic construct
QQHYHYHYHYHQQ

SEQ ID NO: 18 - protein, 11 aa, synthetic construct
HQHYHYHYHQH

SEQ ID NO: 19 - protein, 11 aa, synthetic construct
QYEYHYHYRYQ

SEQ ID NO: 20 - protein, 11 aa, synthetic construct
HYEYHYHYRYH

SEQ ID NO: 21 - protein, 11 aa, synthetic construct
QYEYEQRYRYQ

SEQ ID NO: 22 - protein, 11 aa, synthetic construct
HYEYEYRYRYH

SEQ ID NO: 23 - protein, 11 aa, synthetic construct
QQHYHFHYHQQ

SEQ ID NO: 24 - protein, 11 aa, synthetic construct
HQHYHAHYHQH

SEQ ID NO: 25 - protein, 9 aa, synthetic construct
QHYHAHYHQ

SEQ ID NO: 26 - protein, 11 aa, synthetic construct
QQEYHFHYRQQ

SEQ ID NO: 27 - protein, 11 aa, synthetic construct
HQEYHFHYRQH

SEQ ID NO: 28 - protein, 11 aa, synthetic construct
HQEYHVHYRQH

SEQ ID NO: 29 - protein, 11 aa, synthetic construct
HQEYHIHYRQH

SEQ ID NO: 30 - protein, 11 aa, synthetic construct
QQHFHFHFHQQ

SEQ ID NO: 31 - protein, 11 aa, synthetic construct
HQEFHYHFRQH

SEQ ID NO: 32 - protein, 11 aa, synthetic construct
HQEFHFHFRQH

SEQ ID NO: 33 - protein, 11 aa, synthetic construct
HQEYHCHYRQH

SEQ ID NO: 34 - protein, 11 aa, synthetic construct
QQHYHCHYHQQ

SEQ ID NO: 35 - protein, 11 aa, synthetic construct
QQEYHCHYRQQ

SEQ ID NO: 36 - protein, 8 aa, synthetic construct
GGSGGSGG

SEQ ID NO: 37 - protein, 6 aa, synthetic construct
GGDPGG

SEQ ID NO: 38 - protein, 10 aa, synthetic construct
GGSGDPGSGG

SEQ ID NO: 39 - protein, 6 aa, synthetic construct
HHHHHH

SEQ ID NO: 40 - protein, 5 aa, synthetic construct
PGPGP

SEQ ID NO: 41 - protein, 43 aa, synthetic construct
SKWQHQQDSCRKQLQGVNLTPCEKHIMEKIQGRGDDDDDDDDD

SEQ ID NO: 42 - protein, 37 aa, synthetic construct
LLGDFFRKSKEKIGKEFKRIVQRIKDFLRNLVPRTES

SEQ ID NO: 43 - protein, 17 aa, synthetic construct
GNNRPVYIPQPRPPHPR

SEQ ID NO: 44 - protein, 17 aa, synthetic construct
KWCFRVCYRGICYRRCR

SEQ ID NO: 45 - protein, 8 aa, synthetic construct
SIINFEKL

SEQ ID NO: 46 - protein, 16 aa, synthetic construct
SQAVHAAHAEINEAGR

SEQ ID NO: 47 - protein, 7 aa, synthetic construct
APPHALS

SEQ ID NO: 48 - protein, 31 aa, synthetic construct
LMQYIKANSKFIGIPMGLPQSIALSSLMVAQ

SEQ ID NO: 49 - protein, 12 aa, synthetic construct
AKFVAAWTLKAA

SEQ ID NO: 50 - protein, 60 aa, synthetic construct
MASGGGQQEYHYHYHYRQQPRGQQEYHYHYHYRQQPRGQQEYHYHYHYRQQGGGGPGPGP

SEQ ID NO: 51 - DNA, 300 bp, synthetic construct
  source 1..300: mol_type = other DNA
gcgagcggcgggggtcagcaagagtaccattatcattaccattatcgtcaacagcctagagggcagcaggaatatcactaccactatcattatcggcagcagccgcgcggtcaacaagaatatcactatcattatcattaccgtcagcagggtggcggaggaccaggtccgggcccctaatagcgcaatgctgaaagacccggactagagtggcgagatctatggcgtgtgacccgttatgctccatttcggtcagtgggtcacagctagttgtggattggattgccctaatagctcgat

SEQ ID NO: 52 - protein, 75 aa, synthetic construct
MASGGGQQHFHFHFHQQGGGGQQHFHFHFHQQGGGGQQHFHFHFHQQGGGGQQHFHFHFHQQGGPGPGPHHHHHH

SEQ ID NO: 53 - DNA, 336 bp, synthetic construct
  source 1..336: mol_type = other DNA
gcgagcggaggcggccagcaacattttcactttcattttcaccagcaaggcgggggcggtcagcagcattttcatttccactttcaccagcagggaggtgggggtcaacagcactttcacttccactttcatcagcagggtggcggtggccaacagcatttccatttccacttccatcagcaaggcggtccggggccgggcccacatcatcaccatcatcattgataaaagtttaatcttctatcgcaaggaactgccgatcttaatggatggccggaggtggtatggaagctataagcgcgggtgagagggtaattaggcgtgttcacctacgct

SEQ ID NO: 54 - protein, 73 aa, synthetic construct
MASGGGGQQHYHYHYHQQGGGGQQHYHYHYHQQGGGGQQHYHYHYHQQGGGGQQHYHYHYHQQGGGGGPGPGP

SEQ ID NO: 55 - DNA, 300 bp, synthetic construct
  source 1..300: mol_type = other DNA
gcaagcggcggtgggggacaacagcattaccattatcattatcaccagcaaggcggtgggggtcagcagcattatcattaccactatcatcaacagggtggtggcggacagcagcattaccattaccactatcaccaacagggcggcgggggccaacagcactatcattatcactaccatcagcaaggtggtggcggacctgggccaggaccgtaataggctcgcaatgctgaaagacccggactagagtggcgagatctatggcgtgtgacccgttatgctccatttcggtcagtgggt

SEQ ID NO: 56 - protein, 73 aa, synthetic construct
MASGGGGQQEYHYHYRQQGGGGQQEYHYHYRQQGGGGQQEYHYHYRQQGGGGQQEYHYHYRQQGGGGGPGPGP

SEQ ID NO: 57 - DNA, 300 bp, synthetic construct
  source 1..300: mol_type = other DNA
gcaagcgggggtgggggccaacaggaatatcattatcactatcggcagcaagggggtggcggacagcaagaatatcattatcattaccgccaacagggcggtggcggacaacaggagtatcattaccactaccgtcagcagggcggcgggggacaacaggaataccattatcactatagacagcagggtggtggtggccctggaccgggtccataatagtgacgcaatgctgaaagacccggactagagtggcgagatctatggcgtgtgacccgttatgctccatttcggtcagtgggt

SEQ ID NO: 58 - protein, 73 aa, synthetic construct
MASGGGGHQEYHYHYRQHGGGGHQEYHYHYRQHGGGGHQEYHYHYRQHGGGGHQEYHYHYRQHGGGGGPGPGP

SEQ ID NO: 59 - DNA, 300 bp, synthetic construct
  source 1..300: mol_type = other DNA
gcaagcggaggcgggggtcatcaggagtatcattatcactaccggcagcacggcggtggagggcaccaggaataccattaccattatcgacaacacggtgggggcggacatcaagaatatcactatcattaccgccagcatggaggaggtggccatcaggaatatcattatcactatcgtcaacatgggggcggagggcctggaccaggtccgtaatagttgtggattggattgccattctcctagtgtttttaacgtgacagccgcagggatcccataaaatgcaatcgtagtccacct

SEQ ID NO: 60 - protein, 71 aa, synthetic construct
MASGGGHQEYHVHYRQHGGGGHQEYHVHYRQHGGGGHQEYHVHYRQHGGGGHQEYHVHYRQHGGGDPGPGP

SEQ ID NO: 61 - DNA, 540 bp, synthetic construct
  source 1..540: mol_type = other DNA
gcgagcggaggcggtcaccaggaatatcacgtacattaccgccaacacggcgggggaggccatcaggaatatcatgtgcattatcgtcagcacggaggtgggggccatcaggagtaccatgttcactatagacaacacggcggtggtgggcatcaagaatatcatgtccattaccggcagcatggcggtggtgatccgggtcctggaccataatgaaagtttaatcttctatcgcaaggaactgccgatcttaatggagtgccggaggtggtatggaagctataagcgcgggtgagagggtaattaggcgtgttcacctacgctacgctaacgggcgattctatgatagtaagtagctgaccttagctatcactgtctcaacggcatgcgcaacttgtgaagtgtctactatccttgatatccggaacttgcgatgtaggtaatatgatttaatacatacctggcccagtagttttccaatatgggaacatcaattgtacatcgggccgggataatcatgtcatcacggaagtagccgta

SEQ ID NO: 62 - protein, 65 aa, synthetic construct
MASGGGGQQHYHYHQQGGGGQQHYHYHQQGGGGQQHYHYHQQGGGGQQHYHYHQQGGGGGPGPGP

SEQ ID NO: 63 - DNA, 300 bp, synthetic construct
  source 1..300: mol_type = other DNA
gcatctggcggtgggggccagcagcattatcactatcaccagcaagggggtggtggacaacagcactaccattatcaccagcaaggcggcgggggccaacagcattaccattatcatcagcagggtggtggcggacaacaacattatcattaccatcagcagggtggtggtgggccaggacctgggccgtaatagcgcaatgctgaaagacccggactagagtggcgagatctatggcgtgtgacccgttatgctccatttcggtcagtgggtcacagctagttgtggattggattgccc

SEQ ID NO: 64 - protein, 60 aa, synthetic construct
MASGGGQQHYHYHQQPRGQQHYHYHQQPEGQQHYHYHQQPEGQQHYHYHQQGGGGPGPGP

SEQ ID NO: 65 - DNA, 300 bp, synthetic construct
  source 1..300: mol_type = other DNA
gcgagcggcgggggtcaacagcactaccactaccaccagcaacctcgtggccagcagcattaccattatcatcagcagccggagggtcagcagcattatcattatcaccaacaacctcgtggacaacaacattatcattatcatcagcagggcggagggggtccaggccccggtccgtaatagcgcaatgctgaaagacccggactagagtggcgagatctatggcgtgtgacccgttatgctccatttcggtcagtgggtcacagctagttgtggattggattgccctagtgagtccca

SEQ ID NO: 66 - protein, 63 aa, synthetic construct
MASGGGHQHYHYHQHGGGGHQHYHYHQHGGGGHQHYHYHQHGGGGHQHYHYHQHGGGGPGPGP

SEQ ID NO: 67 - DNA, 300 bp, synthetic construct; identical to SEQ ID NO: 65
  source 1..300: mol_type = other DNA

SEQ ID NO: 68 - protein, 64 aa, synthetic construct
MASGGGQQYHYHYQQGGGGQQYHYHYQQGGGGQQYHYHYQQGGGGQQYHYHYQQGGGGGPGPGP

SEQ ID NO: 69 - DNA, 300 bp, synthetic construct
  source 1..300: mol_type = other DNA
gcgagcggcggcggacagcaatatcactaccattatcaacagggtggcggtgggcaacaatatcactatcattaccagcagggtggcgggggtcaacagtaccattatcattaccaacagggtgggggtggacagcagtatcattatcactatcagcagggcggcggtggcggaccgggacctgggccatgataaaagtttaatcttctatcgcaaggaactgccgatcttaatggatggccggaggtggtatggaagctataagcgcgggtgagagggtaattaggcgtgttcacctac

SEQ ID NO: 70 - protein, 89 aa, synthetic construct
MASGGGQQEYHFHYRQQGGGGQQEYHFHYRQQGGGGQQEYHFHYRQQGGGGQQEYHFHYRQQGGGGQQEYHFHYRQQGPGPGPHHHHHH

SEQ ID NO: 71 - DNA, 336 bp, synthetic construct
  source 1..336: mol_type = other DNA
gcgagcggcggcggtcagcaagaataccacttccactatcgccagcagggagggggcggacagcaggagtatcatttccactatcgtcagcagggtggtgggggccagcaagagtatcactttcactaccgccaacagggtggtggcggccagcaggaataccattttcattaccgtcagcaagggggcggcggtcaacaagaatatcattttcattatcggcagcaaggtccgggaccgggcccacaccatcaccatcatcattgataaaagtttaatcttctatcgcaaggaactgccgatcttaatggatggccggaggtggtatggaagcta

SEQ ID NO: 72 - protein, 89 aa, synthetic construct
MASGGGHQEYHFHYRQHGGGGHQEYHFHYRQHGGGGHQEYHFHYRQHGGGGHQEYHFHYRQHGGGGHQEYHFHYRQHGPGPGPHHHHHH

SEQ ID NO: 73 - DNA, 336 bp, synthetic construct
  source 1..336: mol_type = other DNA
gcgagcggaggcggccaccaggaatatcacttccactaccggcaacatggtggcggtggtcaccaggagtatcatttccactaccgtcagcatggtggcgggggtcatcaagagtatcattttcactatcgccagcacggaggcggtgggcaccaggaataccattttcactaccgccagcatgggggtggcggccatcaggaatatcattttcattatcgtcaacatggcccaggcccgggaccgcatcatcaccatcatcactgataaaagtttaatcttctatcgcaaggaactgccgatcttaatggatggccggaggtggtatggaagcta

SEQ ID NO: 74 - protein, 94 aa, synthetic construct
MASGGGQQEYHYHYHYRQQGGGGQQEYHYHYHYRQQGGGGQQEYHYHYHYRQQGGGDPGGGNNRPVYIPQPRPPHPRLGGGGDPGPGPHHHHHH

SEQ ID NO: 75 - DNA, 300 bp, synthetic construct
  source 1..300: mol_type = other DNA
gcgagcgggggtgggcatcaagagtaccattatcactacagacagcacggaggtggcggacaccaagaatatcattaccattaccggcagcacggcggagggggccatcaggaatatcattatcactatcgacaacatgggggcggcggtcatcaggaataccattatcattatcgtcagcatggcgggggagacccgggtggtggcaacaatcgccctgtttatattcctcagccacgcccgccccacccccgtctgggaggtggtggcgatccgggtccaggtccgtaatagggtgaa

SEQ ID NO: 76 - protein, 122 aa, synthetic construct
MASGGGHQEYHYHYRQHGGGGHQEYHYHYRQHGGGGHQEYHYHYRQHGGGGHQEYHYHYRQHGGGDPGGSKWQHQQDSCRKQLQGVNLTPCEKHIMEKIQGRGDDDDDDDDDGGGGDPGPGP

SEQ ID NO: 77 - DNA, 375 bp, synthetic construct
  source 1..375: mol_type = other DNA
gcgagcggaggaggtcatcaagagtaccattaccattaccgccagcatggcggaggtggtcaccaggaatatcactatcactatcgtcagcatgggggcggagggcaccaggagtatcactatcactatagacagcacggaggcggtggccatcaagaatatcattatcattaccgtcagcatggtgggggcgatccaggtggctctaagtggcaacatcagcaggatagctgtcgcaaacaattacagggcgttaatctgaccccttgcgaaaaacatattatggaaaaaatccaaggccggggtgatgatgacgatgacgacgatgatgacgggggaggtggtgatccgggtcccgggccgtaatagggcaat

SEQ ID NO: 78 - protein, 114 aa, synthetic construct
MASGGGQQHYHYHQQGGGGQQHYHYHQQGGGGQQHYHYHQQGGGGQQHYHYHQQGGGDPGGSKWQHQQDSCRKQLQGVNLTPCEKHIMEKIQGRGDDDDDDDDDGGGGDPGPGP

SEQ ID NO: 79 - DNA, 345 bp, synthetic construct
  source 1..345: mol_type = other DNA
gcgagcggcggcggacaacagcattatcattaccaccaacagggcggtgggggacaacagcattaccactatcatcaacagggtggaggcggacagcagcactatcactaccatcagcagggcgggggcggtcagcaacattatcattatcaccagcaagggggcggtgatcccggaggttctaagtggcaacatcagcaggatagctgtcgtaaacaattacagggagttaatctgaccccttgcgagaaacatatcatggaaaaaattcagggtcgcggtgatgatgacgatgacgacgatgatgacggcggtgggggtgatccggggccaggtccgtaatag

SEQ ID NO: 80 - protein, 97 aa, synthetic construct
MASGGGQQHYHYHYHQQGGGGQQHYHYHYHQQGGGGQQHYHYHYHQQGGGGQQHYHYHYHQQGGGDPGGGNNRPVYIPQPRPPHPRLGGGGDPGPGP

SEQ ID NO: 81 - DNA, 300 bp, synthetic construct
  source 1..300: mol_type = other DNA
gcgagcggtggtggtcaacagcactaccattatcactatcatcagcaagggggaggtgggcagcagcactaccattaccattatcaccaacagggtggtgggggccagcagcattaccattaccactatcatcaacagggcggaggcggccaacagcattatcattatcattatcatcagcagggaggcggagacccgggcggcggtaacaatcggccggtttatattccgcaacctcgcccgccacacccacgtctgggaggtggcggggatccagggcccggtccttaataggtgaac

SEQ ID NO: 82 - protein, 122 aa, synthetic construct
MASGGGQQHYHYHYHQQGGGGQQHYHYHYHQQGGGGQQHYHYHYHQQGGGGQQHYHYHYHQQGGGDPGGSKWQHQQDSCRKQLQGVNLTPCEKHIMEKIQGRGDDDDDDDDDGGGGDPGPGP

SEQ ID NO: 83 - DNA, 375 bp, synthetic construct
  source 1..375: mol_type = other DNA
gcgagcggcggtggacaacagcattatcactatcactatcatcagcaagggggcggcgggcagcaacattatcactaccattatcatcagcagggaggcggtggccagcagcactaccattaccactatcatcaacagggcggtggcggtcagcaacactatcattatcattaccatcaacagggaggaggtgatcccgggggcagcaagtggcagcatcaacaggattcttgccgtaaacagttacagggagttaatctgaccccttgtgagaaacacatcatggaaaaaattcaaggtcgcggagatgatgacgatgacgacgatgatgacggtggtggtggtgatccggggccagggccgtaataggcaatt

SEQ ID NO: 84 - protein, 122 aa, synthetic construct
MASGGGQQEYHYHYRQQGGGGQQEYHYHYRQQGGGGQQEYHYHYRQQGGGGQQEYHYHYRQQGGGDPGGSKWQHQQDSCRKQLQGVNLTPCEKHIMEKIQGRGDDDDDDDDDGGGGDPGPGP

SEQ ID NO: 85 - DNA, 375 bp, synthetic construct
  source 1..375: mol_type = other DNA
gcgagcggcggaggccagcaggaataccattatcactatcgtcagcaagggggtggaggtcagcaggagtaccattaccattatcgccaacagggcggaggcgggcagcaggaatatcactaccactatcgtcagcagggtgggggcggtcagcaagagtatcattatcattatcggcagcaaggaggaggagatccagggggtagcaagtggcaacaccagcaggattcttgcagaaaacaattacaaggtgttaatctgaccccgtgtgaaaaacatattatggaaaaaatccaaggccgcggtgatgatgacgatgacgacgatgatgacggcggcggtggggatcccggtccgggtccttaatagggcaat

SEQ ID NO: 86 - protein, 89 aa, synthetic construct
MASGGGQQHYHYHQQGGGGQQHYHYHQQGGGGQQHYHYHQQGGGGQQHYHYHQQGGGDPGGGNNRPVYIPQPRPPHPRLGGGGDPGPGP

SEQ ID NO: 87 - DNA, 300 bp, synthetic construct
  source 1..300: mol_type = other DNA
gcgagcggagggggccagcagcattatcattaccaccaacaaggtggcgggggacaacaacattaccactatcatcagcagggcgggggtgggcagcaacactatcattaccatcagcagggagggggtggtcaacagcactatcattatcatcagcagggaggaggtgacccgggcggtggtaataaccgcccagtttatattccccagccgagaccaccgcacccgcgtctgggcggtggcggcgatcctggcccaggtccttaatagggtgaaccgcaatgtgactgaaagacccgg

SEQ ID NO: 88 - protein, 97 aa, synthetic construct
MASGGGQQEYHYHYRQQGGGGQQEYHYHYRQQGGGGQQEYHYHYRQQGGGGQQEYHYHYRQQGGGDPGGGNNRPVYIPQPRPPHPRLGGGGDPGPGP

SEQ ID NO: 89 - DNA, 300 bp, synthetic construct
  source 1..300: mol_type = other DNA
gcgagcggcggtggacagcaagaatatcactatcattatcggcagcagggtgggggcggccagcaggaatatcattaccattatcgccagcaagggggcgggggacagcaggagtatcattaccactacagacagcagggtggcggtggccaacaagaataccattatcattatcgtcagcaagggggtggagacccgggtgggggtaacaatcgacccgtttacattccacaaccccgtccgcctcacccgcgcttaggtggtggcggagatccgggacctggcccatgataaaagttt

SEQ ID NO: 90 - protein, 94 aa, synthetic construct; identical to SEQ ID NO: 74

SEQ ID NO: 91 - DNA, 474 bp, synthetic construct
  source 1..474: mol_type = other DNA
gcgagcggcggcggccaacaggagtaccactaccattaccactatcgccagcaaggcggaggagggcaacaggaatatcattaccattatcactatcgccagcagggtggtgggggacagcaggaatatcattatcactatcattatagacaacagggcggtggtgatcctggaggtggtaataaccggcccgtttacattccgcaaccgcgtccgccccatccacgtctgggtgggggcggggaccctggtccgggcccacatcatcatcaccatcactgataagcgagcggcggcggtgggcagcattatcactatcattatcaccaaccgcgcgggcaacactatcactaccattatcatcagccggaaggtcagcattaccattaccactatcatcagccccgtggtcagcattatcattaccactaccatcaaggtggcgggggcggaccaggtcctggaccttgataa

SEQ ID NO: 92 - protein, 88 aa, synthetic construct
MASGGGQQHYHYHQQGGGGQQHYHYHQQGGGGQQHYHYHQQGGGGQQHYHYHQQGGGDPKWCFRVCYRGICYRRCRDPGPGPHHHHHH

SEQ ID NO: 93 - DNA, 336 bp, synthetic construct
  source 1..336: mol_type = other DNA
gcgagcggcggaggacagcagcactatcactaccatcaacagggcggcggtgggcagcagcattaccactaccatcagcagggcggcgggggtcagcaacactaccactatcaccaacagggtgggggcggacagcagcattatcattatcatcaacaaggcggtggtgaccctaaatggtgttttcgcgtgtgttatcgtggcatttgctatcgtcgctgccgggatccaggtccgggtccgcaccatcaccatcatcattgataataaaaagtttaatcttctatcgcaaggaactgccgatcttaatggatggccggaggtggtatggaagct

SEQ ID NO: 94 - protein, 120 aa, synthetic construct
MASGGGQQEYHYHYHYRQQGGGGQQEYHYHYHYRQQGGGGQQEYHYHYHYRQQGGGDPGGGNNRPVYIPQPRPPHPRLGGGGDPGGGNNRPVYIPQPRPPHPRLGGGGDPGPGPHHHHHH

SEQ ID NO: 95 - DNA, 384 bp, synthetic construct
  source 1..384: mol_type = other DNA
gcgagcggaggtggccagcaggaatatcactaccattatcactatcgccagcagggtggcggcgggcaacaagaataccattatcattaccattatcgccagcaaggcggcgggggccaacaggagtatcattaccactatcattaccgtcagcagggaggtggggatccaggcggcggtaacaaccgcccggtgtacatccctcagccgcgcccgcctcacccacggttagggggtggtggtgacccaggtggaggtaataatcgtccggtttatattccgcaaccccgtccacctcatcctcgtttaggcggcggcggagatccgggcccgggtccgcatcatcaccatcaccactaatgaagggtaattaggcgtgttcac

SEQ ID NO: 96 - protein, 127 aa, synthetic construct
MASGGGQQHYHYHQQGGGGQQHYHYHQQGGGGQQHYHYHQQGGGGQQHYHYHQQGGGGQQHYHYHQQGGGDPGGLLGDFFRKSKEKIGKEFKRIVQRIKDFLRNLVPRTESGGGGDPGPGPHHHHHH

SEQ ID NO: 97 - DNA, 573 bp, synthetic construct
  source 1..573: mol_type = other DNA
gcgagcggtggcggccagcagcattatcactatcatcagcagggcggaggcgggcagcagcattaccattatcatcagcaagggggtgggggccagcaacactaccactatcaccagcagggagggggtggccagcagcactatcattaccatcaacaagggggaggtggtcaacaacattatcattatcatcaacaaggtggtggcgatcccggcggtctgttgggcgactttttccgtaagtctaaagaaaaaataggcaaggagtttaaacgcattgttcagcggatcaaagattttcttagaaatttagtgccgcgtaccgaaagcggtggaggaggagatcctggtccaggtccgcatcatcaccaccatcactaatgagcgagcggcggcggtgggcagcattatcactatcattatcaccaaccgcgcgggcaacactatcactaccattatcatcagccggaaggtcagcattaccattaccactatcatcagccccgtggtcagcattatcattaccactaccatcaaggtggcgggggcggaccaggtcctggaccttgataa

SEQ ID NO: 98 - protein, 94 aa, synthetic construct
MASGGGQQHYHYHQQGGGGQQHYHYHQQGGDPGGGNNRPVYIPQPRPPHPPRGGDPGGQQHYHYHQQGGGGQQHYHYHQQGGGPGPGPHHHHHH

SEQ ID NO: 99 - DNA, 474 bp, synthetic construct
  source 1..474: mol_type = other DNA
gcgagcggcggcggccaacagcactatcattatcaccaacagggtggagggggtcaacagcattaccactatcatcagcagggtggcgaccctgggggtggaaataaccggccagtttatattcctcagccgcgcccaccgcatcctccccgtggcggtgatccgggtggtcagcagcattatcattaccaccagcaaggcgggggtgggcagcaacattatcattaccatcagcaaggtggaggccccggaccgggaccacaccatcatcaccaccattaatgagcgagcggcggcggtgggcagcattatcactatcattatcaccaaccgcgcgggcaacactatcactaccattatcatcagccggaaggtcagcattaccattaccactatcatcagccccgtggtcagcattatcattaccactaccatcaaggtggcgggggcggaccaggtcctggaccttgataa

SEQ ID NO: 100 - protein, 145 aa, synthetic construct
MASGGGQQHYHYHQQGGGGQQHYHYHQQGGDPGGGNNRPVYIPQPRPPHPPRGGDPGGQQHYHYHQQGGGGQQHYHYHQQGGDPGGGNNRPVYIPQPRPPHPPRGGDPGGQQHYHYHQQGGGGQQHYHYHQQGGGPGPGPHHHHH

SEQ ID NO: 101 - DNA, 441 bp, synthetic construct
  source 1..441: mol_type = other DNA
gcgagcggagggggacagcagcactatcattatcaccagcagggcggaggcggccaacagcattatcattaccatcaacaagggggcgacccagggggtggtaacaatcgcccagtttacatccctcagccgcgtccgccgcacccaccaagaggaggcgatcctggtggccagcagcactatcattaccatcagcaaggtgggggagggcagcagcactaccattatcaccagcagggtggtgatccgggcggcgggaataaccggcccgtgtacattcctcagccccgtcctccgcacccgccgcgcgggggagatccaggtggtcagcaacattatcattatcaccaacaaggtggaggtggtcaacagcattatcattatcatcaacagggtggtggcccgggccccggccctcaccatcatcaccatcattgataa

SEQ ID NO: 102 - protein, 194 aa, synthetic construct
MASGGGQQHYHYHQQGGGGQQHYHYHQQGGDPGGSKWQHQQDSCRKQLQGVNLTPCEKHIMEKIQGRGDDDDDDDDDPGGGGQQHYHYHQQGGGGQQHYHYHQQGGDPGGSKWQHQQDSCRKQLQGVNLTPCEKHIMEKIQGRGDDDDDDDDDPGGGGQQHYHYHQQGGGGQQHYHYHQQGGGPGPGPHHHHHH

SEQ ID NO: 103 - DNA, 585 bp, synthetic construct
  source 1..585: mol_type = other DNA
gctagtggcggcggtcaacaacactatcactatcatcagcagggcggcggtggccaacagcactatcactaccaccaacaaggtggcgacccgggcgggtcgaagtggcaacaccaacaagatagctgtcgtaaacaactgcaaggcgtgaatctcacaccttgtgagaagcatattatggagaagattcaaggtcgtggagatgacgacgacgacgatgacgacgaccctggtggtggtggtcaacaacactaccattaccaccagcagggcggcggcgggcaacaacattaccattatcaccagcagggcggtgacccaggtggtagcaagtggcagcaccagcaagacagttgccggaagcaacttcaaggcgttaaccttactccctgcgaaaagcacataatggagaaaatccaaggtcgcggcgacgacgatgatgacgacgatgatgatcctggtggcggtggacagcaacactatcattaccatcagcagggaggaggtggccaacaacactatcactaccatcagcaaggtggtgggccgggcccggggccgcaccaccaccatcatcactaataa

SEQ ID NO: 104 - protein, 146 aa, synthetic construct
MASGGDPGGHQEYHYHYRQHGGGGSIINFEKLGGGGHQEYHYHYRQHGGDPGGHQEYHYHYRQHGGGGSIINFEKLGGGGHQEYHYHYRQHGGDPGGHQEYHYHYRQHGGGGSIINFEKLGGGGHQEYHYHYRQHGGDPGGPGPGP

SEQ ID NO: 105 - DNA, 441 bp, synthetic construct
  source 1..441: mol_type = other DNA
gcgagcggcggtgacccgggtgggcatcaggagtatcattatcactatcgccaacatgggggcggtggcagcattataaactttgaaaagcttggcggtggagggcaccaggaatatcattaccattatcgtcagcacggtggagatccagggggtcatcaggaataccattatcactacagacagcacggtgggggtggaagtatcatcaattttgagaaactgggtggtggcgggcatcaggaatatcactatcactatcgccagcatggcggagatcctggaggacatcaggaataccactaccactatcggcaacatggcgggggtggctctattattaatttcgaaaaattaggcggaggcggtcatcaagagtatcattatcattaccgtcaacatggtggggatcctggaggcccaggccccggtccgtaatag

SEQ ID NO: 106 - protein, 166 aa, synthetic construct
MASGGDPGGHQEYHYHYRQHGGGGSIINFEKLGGGGHQEYHYHYRQHGGGGSIINFEKLGGGGHQEYHYHYRQHGGGGSIINFEKLGGGGHQEYHYHYRQHGGGGSIINFEKLGGGGHQEYHYHYRQHGGGGSIINFEKLGGGGHQEYHYHYRQHGGDPGGPGPGP

SEQ ID NO: 107 - DNA, 520 bp, synthetic construct
  source 1..520: mol_type = other DNA
gcgagcggtggcgaccccggtggacatcaggaataccattatcattaccggcaacacggcgggggtggtagtattatcaattttgaaaagcttggcggtgggggtcaccaggaatatcactatcactatcgccagcatgggggcggtggaagcataattaacttcgaaaaactcgggggtggcggacaccaggagtatcattatcactatcgtcagcacggtggcgggggatcaataatcaactttgagaaattaggtgggggcggccatcaagaatatcattaccattaccgccagcacggtgggggcggttctatcattaatttcgagaaactgggcgggggcggacatcaggagtaccattatcactacagacaacatgggggcgggggctcgattattaattttgaaaaactgggtggtggcggtcatcaggaatatcattatcattatcgtcaacatggtggagatccaggtggaccgggaccgggcccttaatgaaagtttaatcttctatcgc

SEQ ID NO: 108 - protein, 73 aa, synthetic construct
MASGGDPGGHQEYHYHYRQHGGGGHQEYHYHYRQHGGGGSIINFEKLGGGGHQEYHYHYRQHGGDPGGPGPGP

SEQ ID NO: 109 - DNA, 300 bp, synthetic construct
  source 1..300: mol_type = other DNA
gcgagcggcggcgaccctggtgggcaccaggaataccattatcattaccgtcaacatggcgggggcggtcaccaggaatatcattaccattatcggcagcatggcgggggtggaagcatcattaattttgagaaactgggtgggggcggccaccaagaatatcattatcattatcgccagcacggtggagatccgggtggtcccggaccgggaccatgataaaagtttaatcttctatcgcaaggaactgccgatcttaatggatggccggaggtggtatggaagctataagcgcgggtg

SEQ ID NO: 110 - protein, 88 aa, synthetic construct
MASGGDPGGQQEYHYHYRQQGGGGQQEYHYHYRQQGGGGSIINFEKLGGGGQQEYHYHYRQQGGGGQQEYHYHYRQQGGDPGGPGPGP

SEQ ID NO: 111 - DNA, 400 bp, synthetic construct
  source 1..400: mol_type = other DNA
gcgagcggtggcgacccgggtggacaacaggaatatcattaccattatcgtcagcagggcggtgggggccagcaggagtaccactatcactacagacaacagggcggtggtggaagcatcattaattttgaaaaactgggcgggggcggacagcaggagtatcattaccactatcggcaacagggtgggggtggacagcaagaatatcattatcattatcgccaacaaggtggagatccgggtggccctgggccaggcccctaatgaaagtttaatcttctatcgcaaggaactgccgatcttaatggatggccggaggtggtatggaagctataagcgcgggtgagagggtaattaggcgtgttcacctacgctacgctaacgggcgattctataagat

SEQ ID NO: 112 - protein, 80 aa, synthetic construct
MASGGDPGGQQHYHYHQQGGGGQQHYHYHQQGGGGSIINFEKLGGGGQQHYHYHQQGGGGQQHYHYHQQGGDPGGPGPGP

SEQ ID NO: 113 - DNA, 300 bp, synthetic construct
  source 1..300: mol_type = other DNA
gcgagcggcggagacccgggtggacaacagcattatcattaccatcaacaagggggtggtggacagcaacattaccactatcatcaacagggtggtggtgggagcattatcaattttgaaaaactgggcggtgggggccagcaacactatcattatcaccagcagggtggtggcggacagcagcattatcactaccatcagcagggcggcgatcctgggggcccaggcccgggaccctgataaaagtttaatcttctatcgcaaggaactgccgatcttaatggatggccggaggtggta

SEQ ID NO: 114 - protein, 134 aa, synthetic construct
MASGGDPGGQQHYHYHQQGGGGSIINFEKLGGGGQQHYHYHQQGGDPGGQQHYHYHQQGGGGSIINFEKLGGGGQQHYHYHQQGGDPGGQQHYHYHQQGGGGSIINFEKLGGGGQQHYHYHQQGGDPGGPGPGP

SEQ ID NO: 115 - DNA, 421 bp, synthetic construct
  source 1..421: mol_type = other DNA
gcgagcgcaagcggcggtgatccgggcggccaacagcactatcattaccatcagcaagggggtgggggatctatcatcaatttcgaaaagcttggcggcgggggtcagcagcattatcactatcatcagcagggtggtgatccaggtggacagcagcactaccactatcaccagcagggtgggggtggctccataattaactttgagaaactgggcggtggcggacagcagcattaccattatcatcagcaaggcggagaccctggcggccagcagcactatcactaccatcaacaagggggtggcgggtcaattattaattttgaaaaattaggtggcggtggtcaacagcattatcattatcatcaacaaggtggagatcccgggggacccgggcctggaccgtaatgaaagtttaatc

SEQ ID NO: 116 - protein, 200 aa, synthetic construct
MASGGDPGGQQEYHYHYRQQGGGGSIINFEKLGGGGQQEYHYHYRQQGGGGAPPHALSGGGGQQEYHYHYRQQGGGGLMQYIKANSKFIGIPMGLPQSIALSSLMVAQGGGGQQEYHYHYRQQGGGGAKFVAAWTLKAAGGGGQQEYHYHYRQQGGGGSQAVHAAHAEINEAGRGGGGQQEYHYHYRQQGGDPGGPGPGP

SEQ ID NO: 117 - DNA, 603 bp, synthetic construct
  source 1..603: mol_type = other DNA
gcgagcggcggtgaccctggcgggcagcaggaatatcactaccactatcgccaacagggaggtggtggctccattataaattttgaaaaactagggggtggtggacagcaggaatatcattaccactatcgccagcaagggggcggcggtgcgcctccacatgcccttagtggcggagggggtcagcaagagtatcattatcattaccgacagcagggtggcggaggtctcatgcaatatattaaagcgaattcgaaattcatcggcatacccatgggtttgccgcagagcatcgcactgtcttcactgatggtggcacaaggtggcggcggtcagcaggaataccactaccattatagacagcaaggaggtggcggggctaaatttgtcgctgcctggaccttaaaggctgcaggcgggggcgggcaacaggagtatcactaccactatcggcagcagggaggagggggaagccaggccgttcatgcggcacatgcggagattaacgaagccggccgtggcggtgggggtcagcaagaatatcattatcattaccgtcaacaagggggagatccgggtggtccgggaccaggtccctgataa

SEQ ID NO: 118 - DNA, 5370 bp, synthetic construct
  source 1..5370: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgatatcgccatggaaccgcgtggcaccagggtacccagatctgggctgtccatgtgctggcgttcgaatttagcagcagcggtttctttcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 119 - protein, 22 aa, synthetic construct
QQHYHYHQQGGGGQQHYHYHQQ